(19) United States
(12) Patent Application Publication
(10) Pub. No.: US 2023/0340530 A1
(43) Pub. Date: Oct. 26, 2023

(54) Methods of Protecting RNA

(71) Applicant: Pfizer Inc., New York, NY (US)

(72) Inventors: Timothy Leyden Foley, Mystic, CT (US); Christopher John Helal, Somerville, MA (US)

(73) Assignee: Pfizer Inc., New York, NY (US)

(21) Appl. No.: 18/041,447

(22) PCT Filed: Aug. 27, 2021
(86) PCT No.: PCT/IB2021/057863

§ 371 Date: Feb. 13, 2023

Related U.S. Application Data
(60) Provisional application No. 63/227,098, filed on Jul. 29, 2021.
(60) Provisional application No. 63/072,778, filed on Aug. 31, 2020.

(51) Int. Cl. C12N 15/86; C12N 15/113

(52) U.S. Cl. CPC C12N 15/86; C12N 15/113; C12N 2310/11; C12N 2320/51; C12N 2320/52; C12N 2770/24243

(57) ABSTRACT

The present invention provides methods of protecting ribonucleic acid (RNA) from degradation under certain conditions by contacting the RNA with compounds that contain at least one boron atom. The present invention also provides compositions comprising such compounds and RNA.

### FIELD OF THE INVENTION

The present invention provides methods of protecting ribonucleic acid (RNA) from degradation by contacting the RNA with a compound that contains at least one boron atom (boron compound) and compositions comprising the boron compound and RNA.

### BACKGROUND OF THE INVENTION

Over the last 30 years, RNA has been shown to be capable of producing therapeutic effects in cells and complex organisms, and recently, RNA-based drugs have been approved for use in subjects. RNA’s potential as a therapeutic agent is an active area of biomedical research, and this area is expected to mature rapidly in the coming years. To date, RNA has been used to target nucleic acids and proteins, and to encode proteins. Single-strand antisense oligonucleotides and double-strand interfering RNA’s can prevent protein synthesis by preventing transcription and/or translation. RNA can also alter protein function directly by forming aptamers to control protein activity, thereby realizing a therapeutic goal related to altering protein function. Finally, messenger RNAs (mRNAs) can be used to produce desired proteins, for example, two important therapeutic uses of mRNAs are as vaccines or for protein replacement (Sarah Deweerdt, Nature, Vol. 574, 17 Oct. 2019).

While it is clear that RNA can have powerful and beneficial therapeutic effects, a major challenge to its use involves manufacture, dosing and delivery. (Kenneth Lundstrom, Gene Therapy, Vol. 27, 183-185, 2020). RNA is highly susceptible to the ever-ubiquitous RNAse, an enzyme that biological organisms produce and secrete in high abundance to degrade foreign RNA. In addition to biochemical attack, RNA is prone to acid or base-mediated chemical degradation, especially at the 2′ hydroxyl group in the phosphate backbone; the rate of this chemical degradation follows first order kinetics and is highly influenced by temperature. With regards to dosing and delivery, RNA is a macromolecule and is therefore unable to cross cell membranes readily via simple diffusion. RNA likely cannot be administered alone, either orally or via injection, as the body will rapidly clear the RNA before any therapeutic effect can be produced at the intended site of action. Effective delivery involves allowing the RNA to reach the desired site of action and allowing it to enter the cell by processes beyond simple diffusion. Delivery vehicles, such as lipid nanoparticles that effectively encapsulate the RNA, must be used to deliver RNA into cells, typically via endocytosis, to achieve the desired effect. These lipoidal formulations have shown to be effective for in vivo and in vitro delivery, however they can pose problems to the RNA therapeutic itself. Specifically, when RNA is formulated with commonly used lipoidal excipients to create nanoparticles, liposomes, or micelles, the RNA can experience a highly basic local environment resulting in chemical degradation of the RNA. Amelioration of this degradation is typically achieved by short latency between formulation and dosing, or -80° C. cold storage, and thus a need exists to overcome these degradation issues. Discovering ways to protect RNA from chemically mediated RNA degradation is currently an unmet challenge where solutions would have a large benefit towards the manufacture, dosing, and delivery of RNA. Furthermore, the discovery of methods that protect RNA from degradation would enable research groups world-wide to evaluate and develop RNA as a viable therapeutic for treating a variety of conditions/disorders in subjects in need of such treatment.

The present invention provides methods for protecting a wide variety of RNAs from degradation by contacting the RNAs in need of such protection to compounds that contain at least one boron atom. The present invention further provides compositions comprising a wide variety of nucleic acids such as RNAs and compounds that contain at least one boron atom.

### SUMMARY OF THE INVENTION

The present invention provides methods of protecting RNA from degradation by contacting the RNA with a compound that contains at least one boron atom.

The present invention provides compositions comprising a boron compound and a nucleic acid such as an RNA or a DNA.

The present invention provides compositions comprising a boron compound and an RNA contained within a non-viral vector.

The present invention provides compositions comprising a boron compound and an RNA contained within a viral vector.

### BRIEF DESCRIPTION OF THE FIGURES

FIG. 1 provides a schematic representation and general description for the RNA Fluorescence Polarization (FP) protection assay.

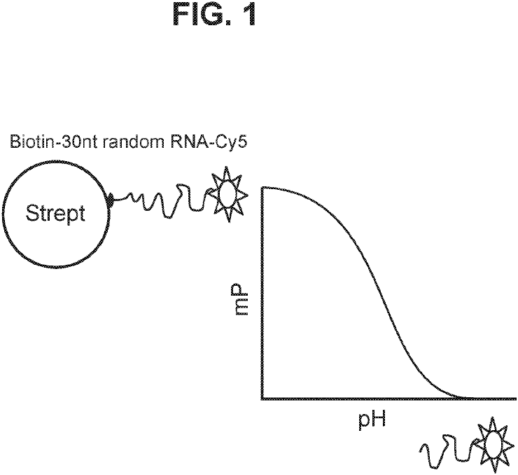

FIG. 2 provides the RNA FP protection assay plot generated for PF-07230998. The y-axis percent activity represents the ability of the boron compound PF-07230998 to protect RNA from being cleaved/degraded. The x-axis Log Concentration [M] represents the concentration of boron compound. The circles and triangles represent replicate measurements of fluorescence polarization % activity for each dose of boron (i.e. n=2).

FIG. 3 provides the RNA FP protection counter screen assay plot generated for PF-07230998. The lack of activity (y-axis) provides support that the RNA protective effect demonstrated in FIG. 2 for boron compound PF-07230998 is not a false positive. The x-axis Log Concentration [M] represents the concentration of boron compound. The circles and triangles represent replicate measurements of fluorescence polarization % activity for each dose of boron (i.e. n=2).

FIG. 4 provides the RNA FP protection assay plot generated for PF-06967658. The y-axis percent activity represents the ability of the boron compound PF-06967658 to protect RNA from being cleaved/degraded. The x-axis Log Concentration [M] represents the concentration of boron compound. The circles and triangles represent replicate measurements of fluorescence polarization % activity for each dose of boron (i.e. n=2).

FIG. 5 provides the RNA FP protection counter screen assay plot generated for PF-06967658. The lack of activity (y-axis) provides support that the RNA protective effect demonstrated in FIG. 4 for boron compound PF-06967658 is not a false positive. The x-axis Log Concentration [M] represents the concentration of boron compound. The circles and triangles represent replicate measurements of fluorescence polarization % activity for each dose of boron (i.e. n=2).

FIG. 6 provides the RNA FP protection assay plot generated for PF-06966741. The y-axis percent activity represents the ability of the boron compound PF-06966741 to protect the RNA from being cleaved/degraded. The x-axis Log Concentration [M] represents the concentration of boron compound. The circles and triangles represent replicate measurements of fluorescence polarization % activity for each dose of boron (i.e. n=2).

FIG. 7 provides the RNA FP protection counter screen assay plot generated for PF-06966741. The lack of activity (y-axis) provides support that the RNA protective effect demonstrated in FIG. 6 for boron compound PF-06966741 is not a false positive. The x-axis Log Concentration [M] represents the concentration of boron compound. The circles and triangles represent replicate measurements of fluorescence polarization % activity for each dose of boron (i.e. n=2).

FIG. 8 provides a schematic representation of the syto-9 dye intercalation assay. For boron compounds that protect RNA, the syto-9 dye intercalation assay will: (1) produce a signal in the primary assay with an EC₅₀ <300 µM; and (2) produce no signal in the counter screen assay with an EC₅₀ >300 µM.

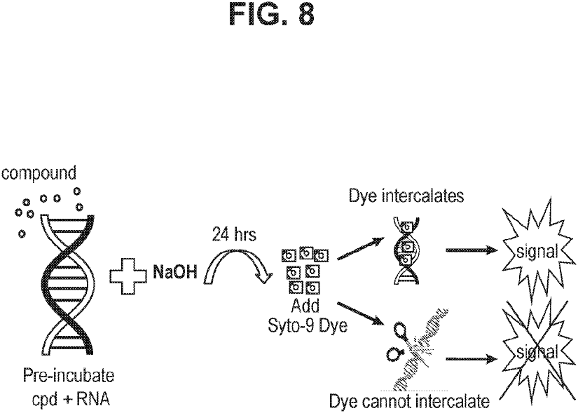

FIG. 9 provides the syto-9 dye intercalation assay plots generated for: PF-06963078 and Fgenl RNA (FIG. 9A); PF-06963078 and CHI-18 RNA (FIG. 9B); and PF-06963078′s counter screen plot (FIG. 9C).

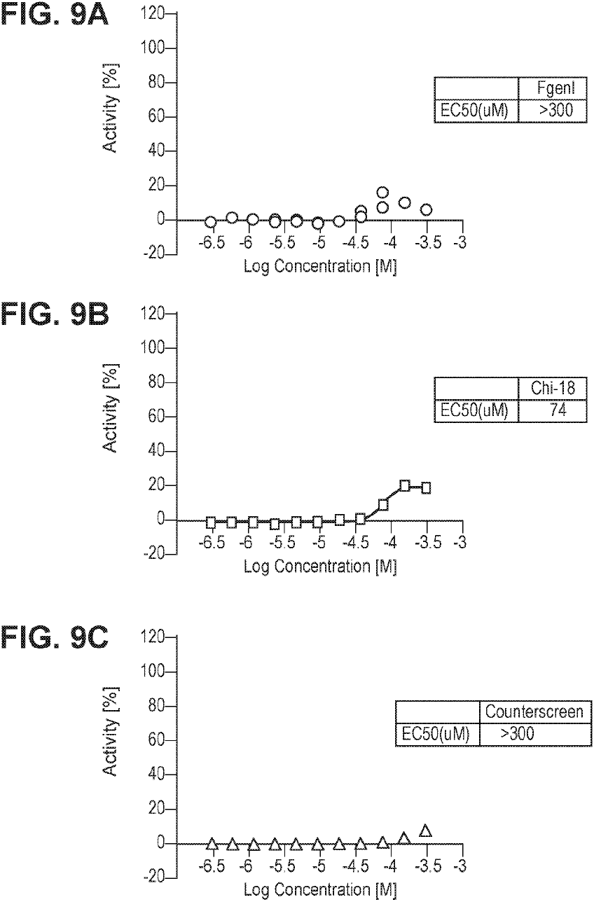

FIG. 10 provides the syto-9 dye intercalation assay plots generated for: PF-06962739 and Fgenl RNA (FIG. 10A); PF-06962739 and CHI-18 RNA (FIG. 10B); and PF-06962739′s counter screen plot (FIG. 10C).

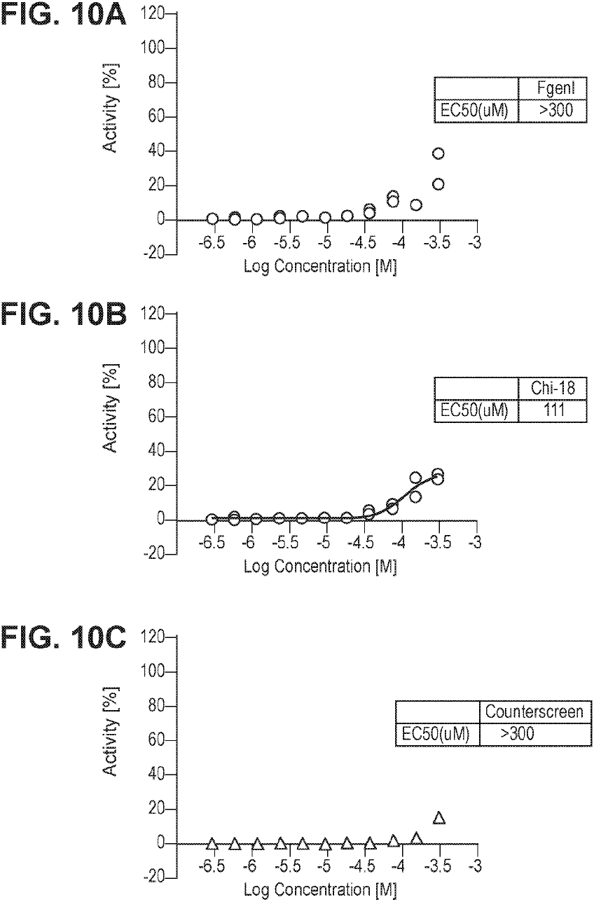

FIG. 11 provides the syto-9 dye intercalation assay plots generated for: PF-06963029 and Fgenl RNA (FIG. 11A); PF-06963029 and CHI-18 RNA (FIG. 11B); and PF-06963029′s counter screen plot (FIG. 11C).

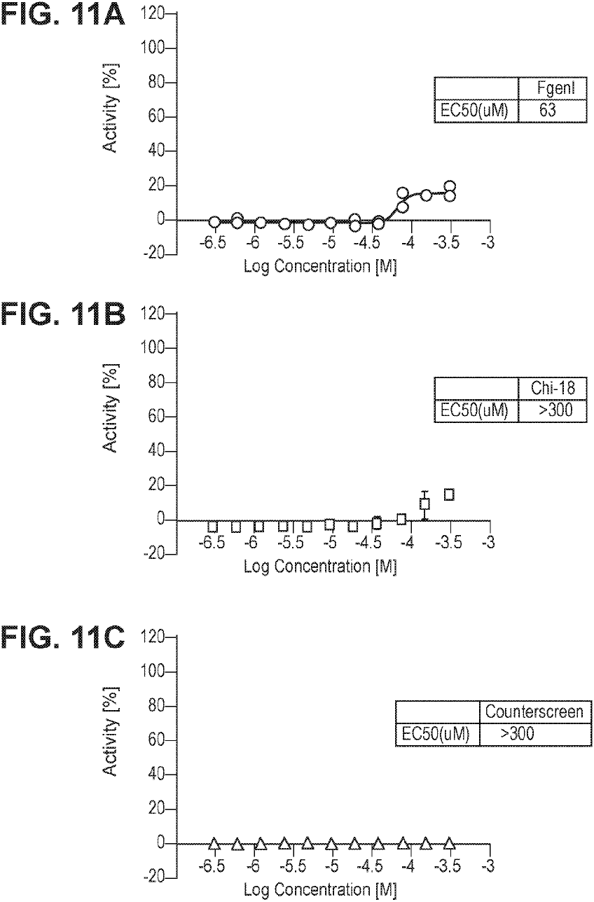

FIG. 12 provides the syto-9 dye intercalation assay plots generated for: PF-06962691 and Fgenl RNA (FIG. 12A); PF-06962691 and CHI-18 RNA (FIG. 12B); and PF-06962691′s counter screen plot (FIG. 12C).

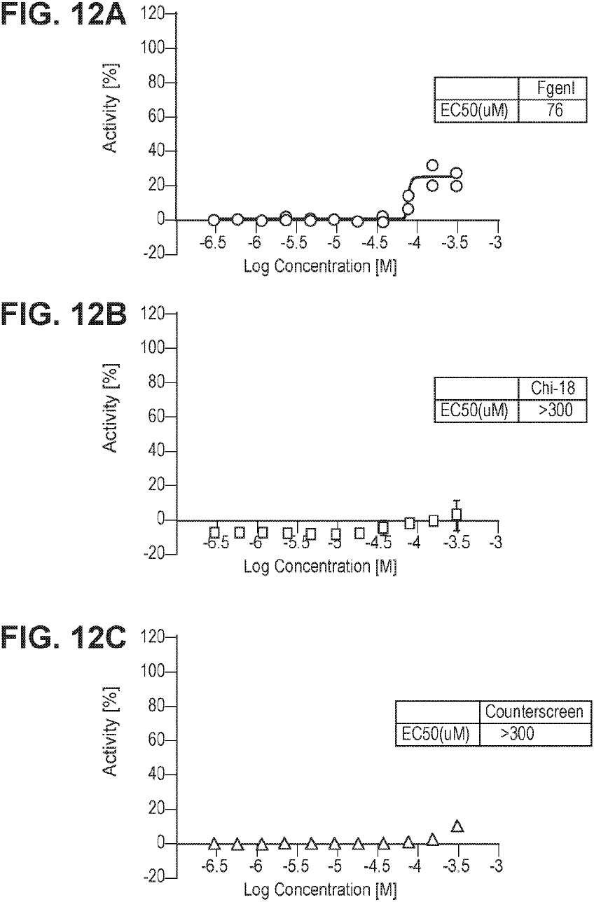

FIG. 13 provides droplet digital polymerase chain reaction assay plots generated for: PF-06957640 and CHI-18 RNA (FIG. 13A); and PF-01499456 and CHI-18 RNA (FIG. 13B); both at pH 10.4 and 30° C. for 16 hours.

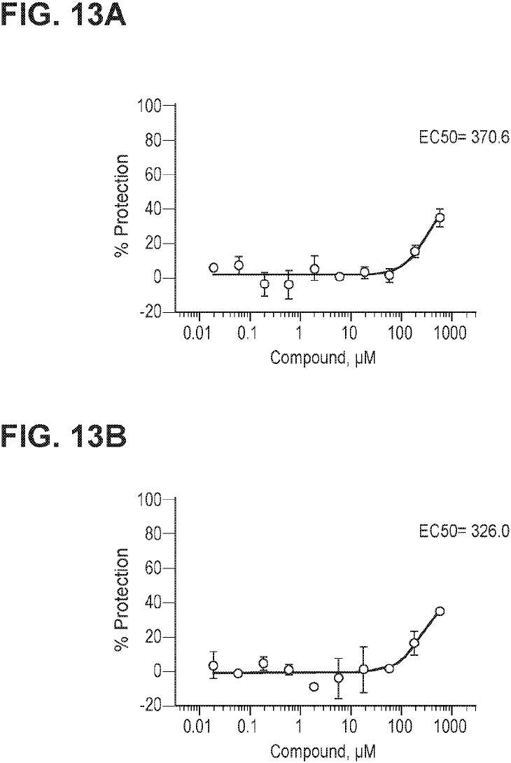

FIG. 14 provides the droplet digital polymerase chain reaction assay plots generated for: PF-06959336 and CHI-18 RNA (FIG. 14A); PF-06957640 and CHI-18 RNA (FIG. 14B); and PF-01499456 and CHI-18 RNA (FIG. 14C); all at pH 11.4 and 30° C. for 16 hours.

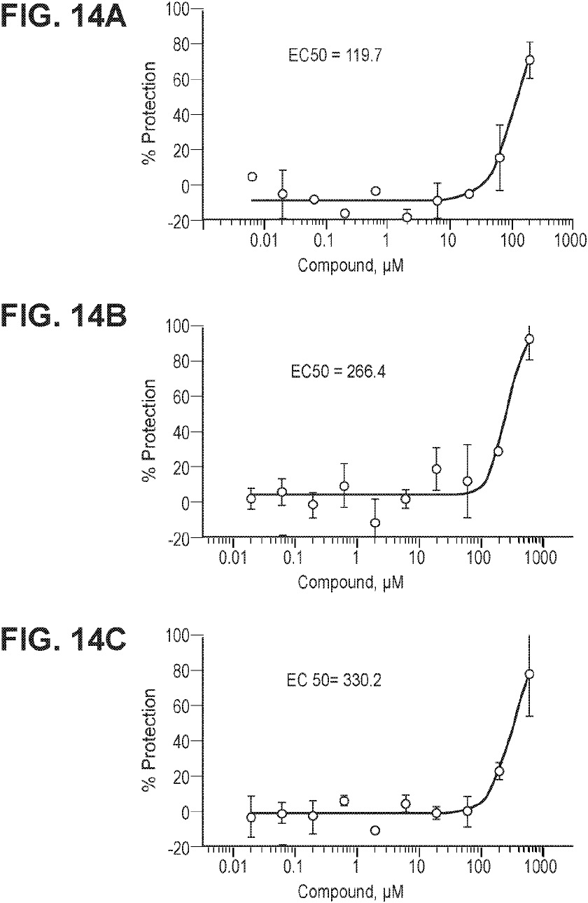

FIG. 15 provides droplet digital polymerase chain reaction assay plots generated for: PF-06957640 and HCV RNA (FIG. 15A); and PF-01499456 and HCV RNA (FIG. 15B); both at pH 11.4 and 42° C. for 16 hours.

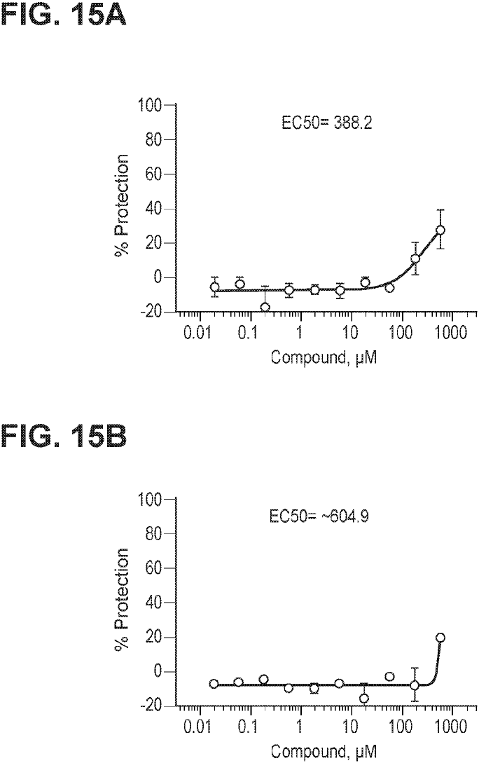

FIG. 16 provides TapeStation assay plots generated for: PF-06957640 and CHI-18 RNA at time 0, 0° C., pH 11.4; PF-06957640 (200 µM) and CHI-18 RNA at 8 minutes, 78° C., pH 11.4; and PF-06957640 (600 µM) and CHI-18 RNA at 8 minutes, 78° C., pH 11.4. A shift to smaller size in this plot indicates degradation. Y-axis is the sample intensity (normalized FU) [10³]; X-axis is the number of intact nucleotides. The untreated plot is non-degraded Chi18 RNA + compounds or DMSO, and is not heated.

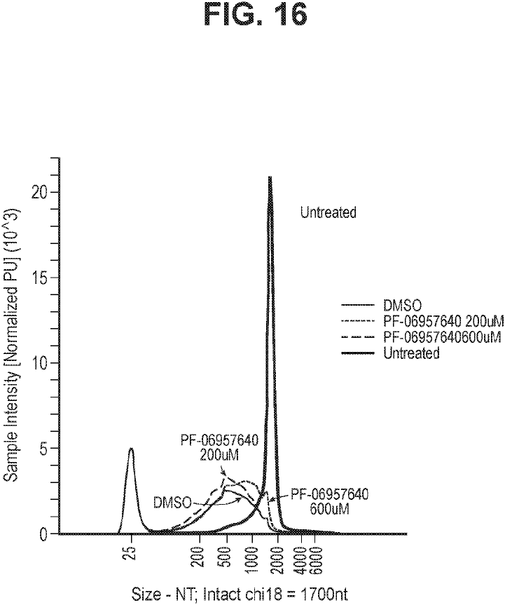

FIG. 17 provides TapeStation assay plots generated for: PF-06957640 and Fgenl RNA at time 0, 0° C., pH 11.4; and PF-06957640 (1.2 mM) and Fgenl RNA at 5 minutes, 65° C., pH 11.4. Y-axis is the sample intensity [normalized FU] (10³); X-axis is the number of intact nucleotides.

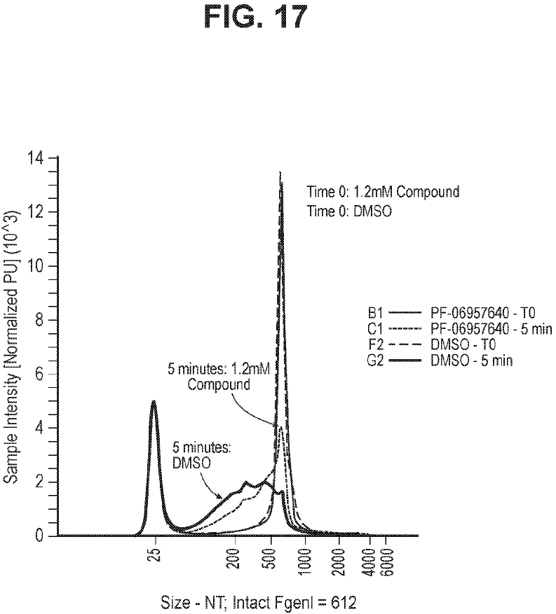

FIG. 18 provides TapeStation assay plots generated for: PF-06957640 and Fgenl RNA at time 0, 0° C., pH 11.4; and PF-06957640 (1.2 mM) and Fgenl RNA at 10 minutes, 65° C., pH 11.4. Y-axis is the sample intensity [normalized FU] (10³); X-axis is the number of intact nucleotides.

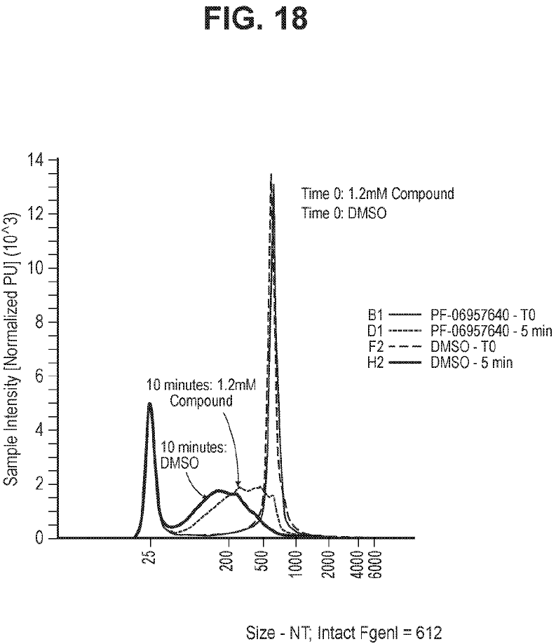

FIG. 19 provides TapeTtation assay plots generated for: PF-01499456 and Fgenl RNA at time 0, 0° C., pH 11.4; and PF-01499456 (1.2 mM) and Fgenl RNA at 5 minutes, 65° C., pH 11.4. Y-axis is the sample intensity [normalized FU] (10³); X-axis is the number of intact nucleotides.

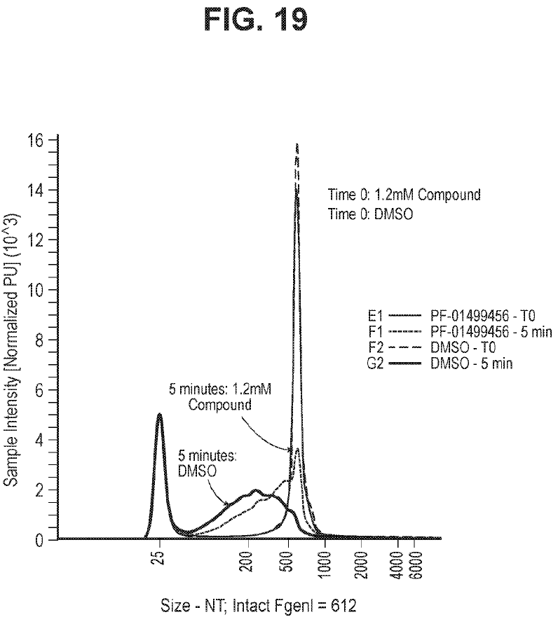

FIG. 20 provides TapeStation assay plots generated for: PF-01499456 and Fgenl RNA at time 0, 0° C., pH 11.4; and PF-01499456 (1.2 mM) and Fgenl RNA at 10 minutes, 65° C., pH 11.4. Y-axis is the sample intensity [normalized FU] (10³); X-axis is the number of intact nucleotides.

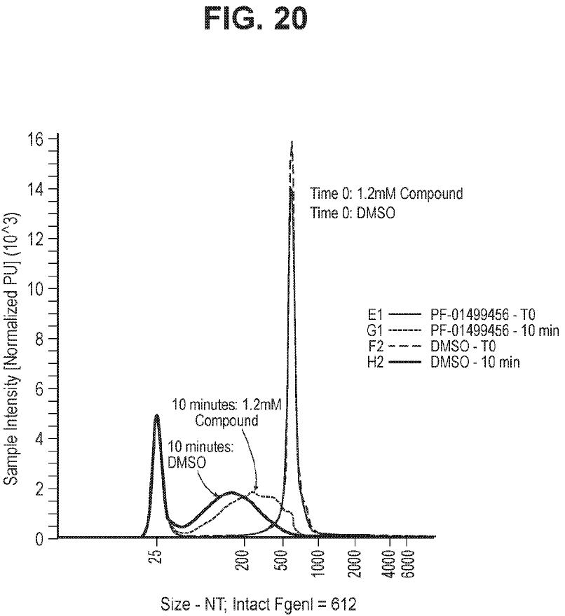

FIG. 21 provides TapeStation assay plots generated for: PF-06957808 and Fgenl RNA at time 0, 0° C., pH 11.4; and PF-06957808 (1.2 mM) and Fgenl RNA at 5 minutes, 65° C., pH 11.4. Y-axis is the sample intensity [normalized FU] (10³); X-axis is the number of intact nucleotides.

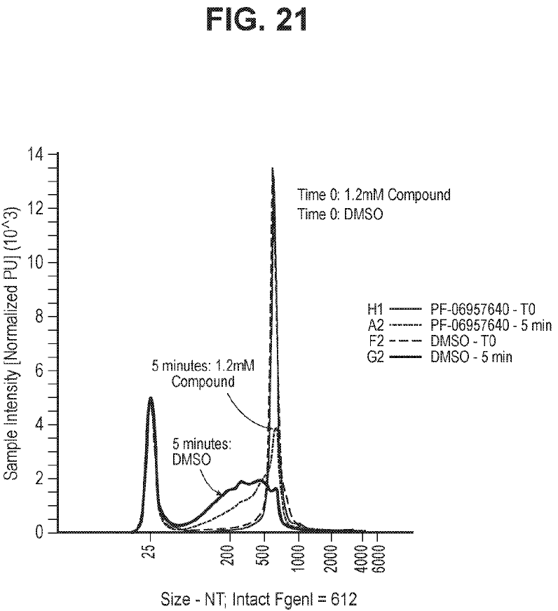

FIG. 22 provides TapeStation assay plots generated for: PF-06957808 and Fgenl RNA at time 0, 0° C., pH 11.4; and PF-06957808 (1.2 mM) and Fgenl RNA at 10 minutes, 65° C., pH 11.4. Y-axis is the sample intensity [normalized FU] (10³); X-axis is the number of intact nucleotides.

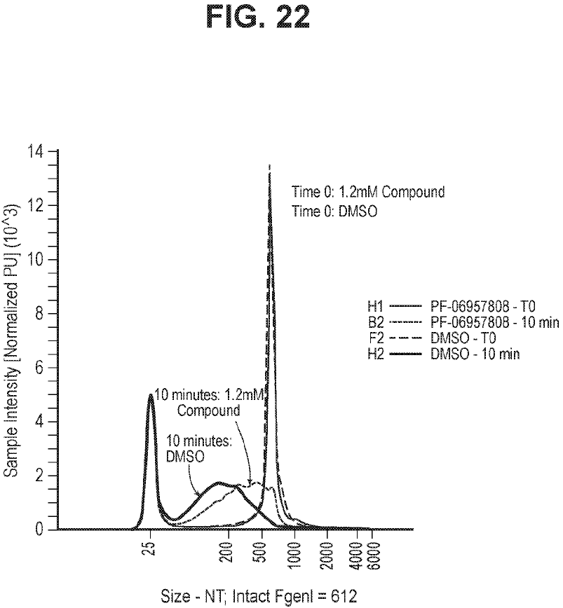

FIG. 23 provides TapeStation assay plots generated for: PF-06957880 and Fgenl RNA at time 0, 0° C., pH 11.4; and PF-06957880 (1.2 mM) and Fgenl RNA at 5 minutes, 65° C., pH 11.4. Y-axis is the sample intensity [normalized FU] (10³); X-axis is the number of intact nucleotides.

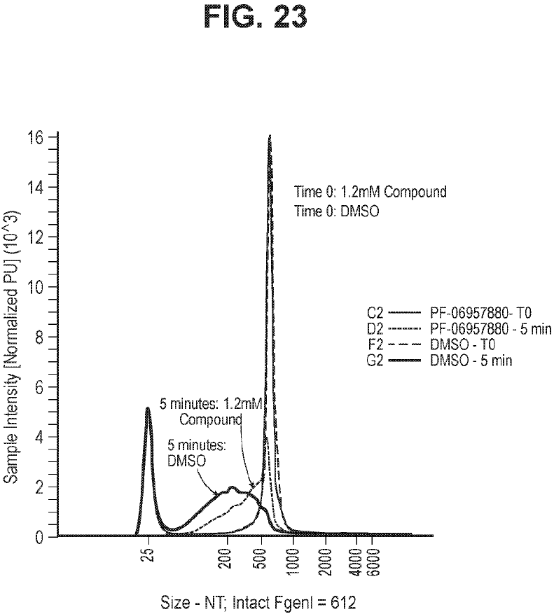

FIG. 24 provides TapeStation assay plots generated for: PF-06957880 and Fgenl RNA at time 0, 0° C., pH 11.4; and PF-06957880 (1.2 mM) and Fgenl RNA at 10 minutes, 65° C., pH 11.4. Y-axis is the sample intensity [normalized FU] (10³); X-axis is the number of intact nucleotides.

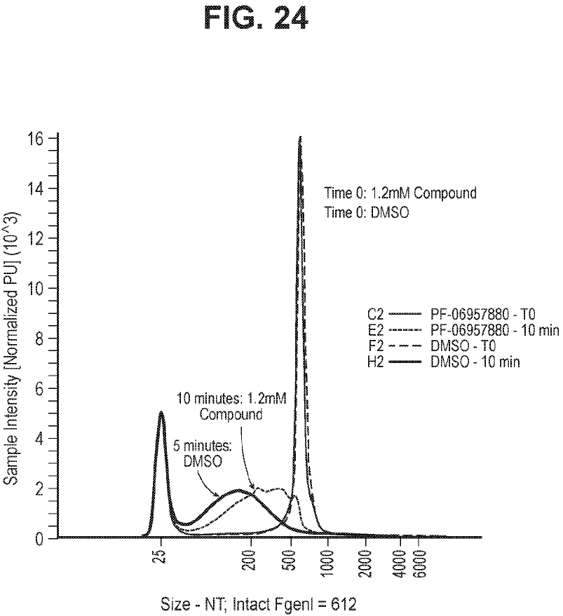

FIG. 25 provides TapeStation assay plots generated for: PF-06959336 and Fgenl RNA at time 0, 0° C., pH 11.4; and PF-06959336 (1.2 mM) and Fgenl RNA at 5 minutes, 65° C., pH 11.4. Y-axis is the sample intensity [normalized FU] (10³); X-axis is the number of intact nucleotides.

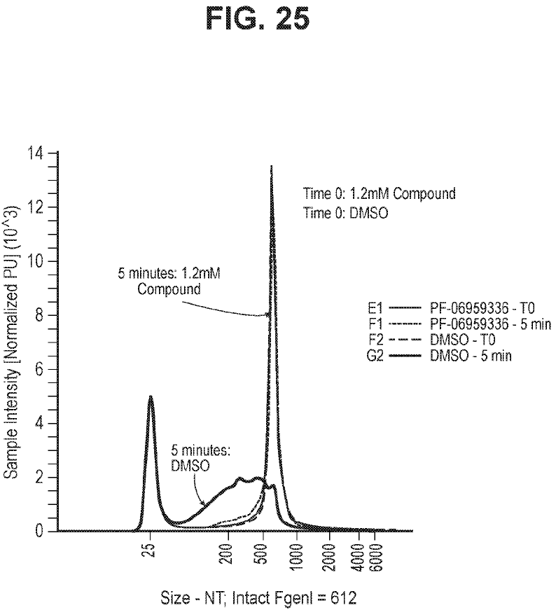

FIG. 26 provides TapeStation assay plots generated for: PF-06959336 and Fgenl RNA at time 0, 0° C., pH 11.4; and PF-06959336 (1.2 mM) and Fgenl RNA at 10 minutes, 65° C., pH 11.4. Y-axis is the sample intensity [normalized FU] (10³); X-axis is the number of intact nucleotides.

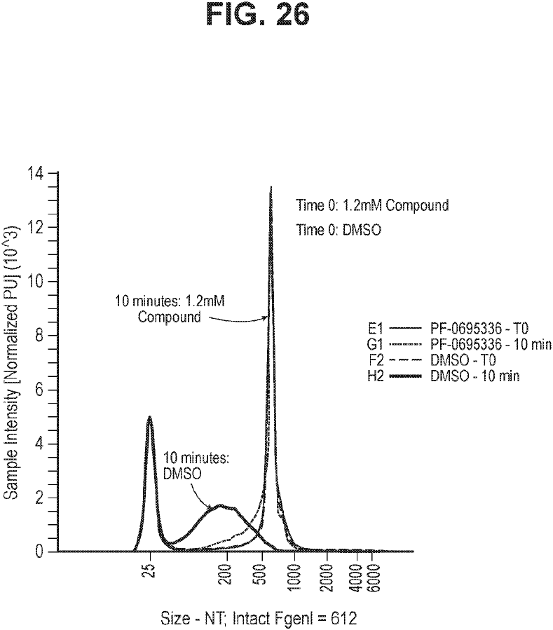

FIG. 27 provides TapeStation assay plots generated for: PF-06957807 and Fgenl RNA at time 0, 0° C., pH 11.4; and PF-06957807 (1.2 mM) and Fgenl RNA at 5 minutes, 65° C., pH 11.4. Y-axis is the sample intensity [normalized FU] (10³); X-axis is the number of intact nucleotides.

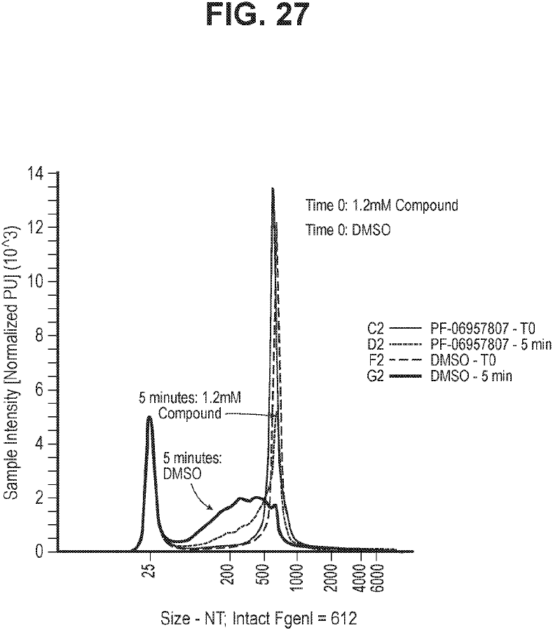

FIG. 28 provides TapeStation assay plots generated for: PF-06957807 and Fgenl RNA at time 0, 0° C., pH 11.4; and PF-06957807 (1.2 mM) and Fgenl RNA at 10 minutes, 65° C., pH 11.4. Y-axis is the sample intensity [normalized FU] (10³); X-axis is the number of intact nucleotides.

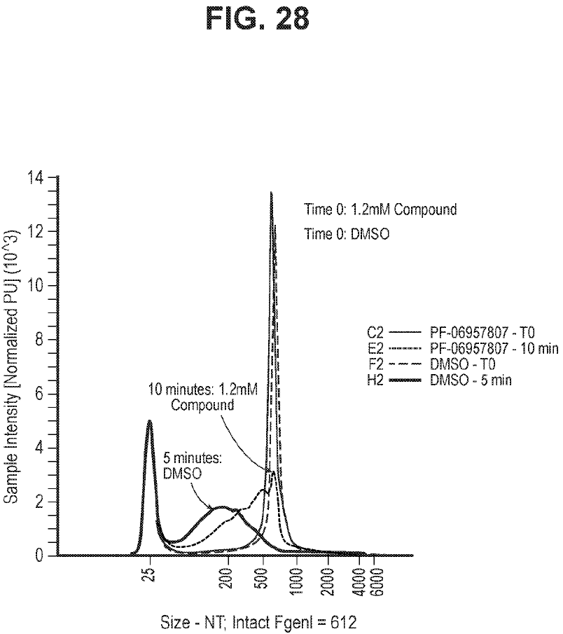

FIG. 29 provides Fgenl RNA degradation results via gel electrophoresis for: Fgenl and PF-06957640 (600 µM); Fgenl and PF-06959336 (600 µM); Fgenl and PF-06957808 (600 µM); Fgenl and PF-06957807 (600 µM); Fgenl and DMSO; at 0 minutes, 5 minutes, 10 minutes, 65° C., pH 11.4.

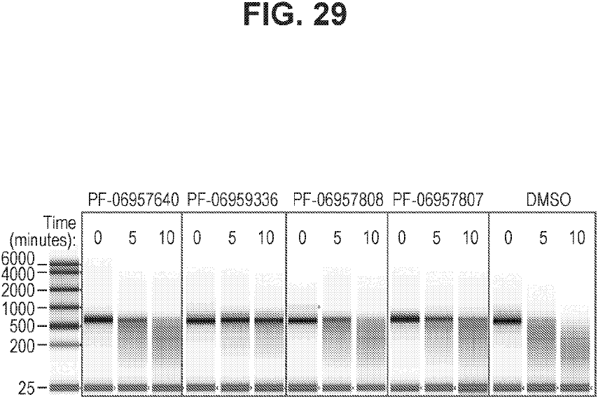

FIG. 30 provides a graphical representation of Fgenl RNA degradation in the presence of several boron containing compounds (1.2 mM) for: Fgenl and PF-06957640; Fgenl and PF-06959336; Fgenl and PF-06957808; Fgenl and PF-06957807; Fgenl and DMSO; at 0 minutes, 5 minutes, 10 minutes, 65° C., pH 11.4.

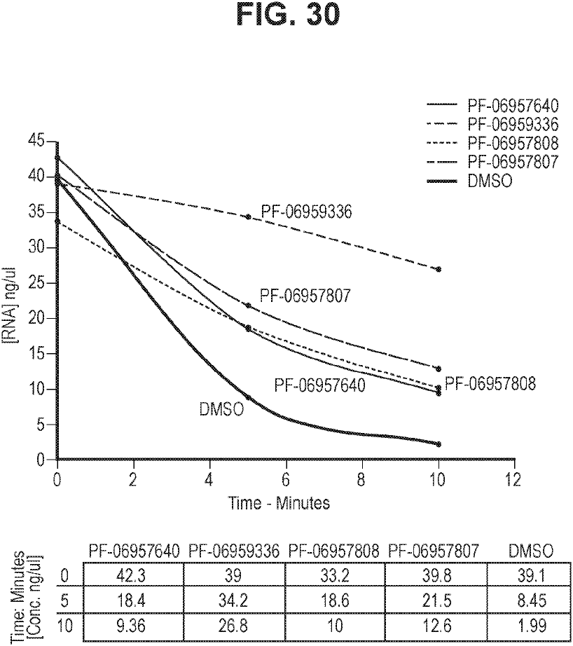

FIG. 31 provides results from the TapeStation assay with Fgenl RNA at 55° C. for 15 minutes, 50 mM Tris at pH 11, using compounds PF-06959336, PHA-00804319, PF-06961361, and PF-06956816, each at 1 mM, 0.33 mM, and 0.11 mM.

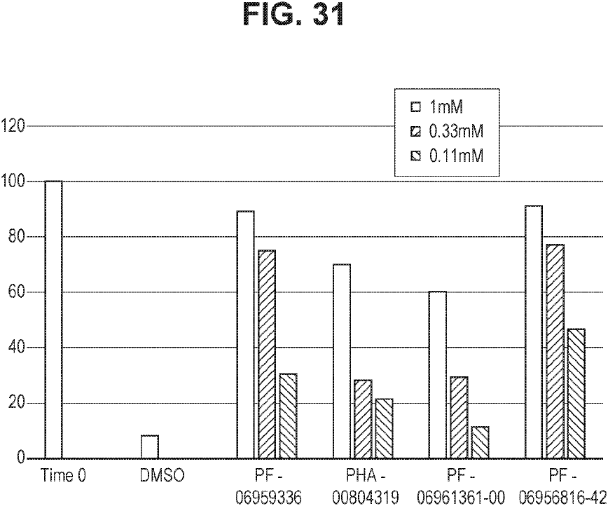

FIG. 32 provides results from the TapeStation assay with chi18 RNA at 55° C. for 15 minutes, 50 mM Tris at pH 11, using compounds PF-06959336, PHA-00804319, PF-06961361, and PF-06956816, each at 1 mM, 0.33 mM, and 0.11 mM.

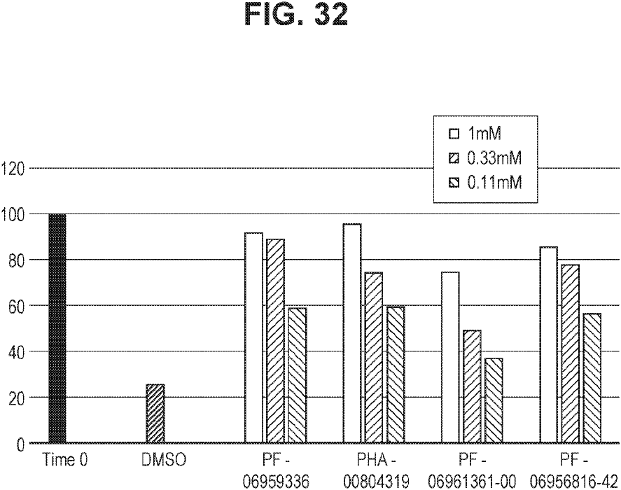

### DETAILED DESCRIPTION OF THE INVENTION

Described below are a number of embodiments (E) of the present invention.

E1. According to a first aspect of the present invention, there is provided a method of protecting an RNA from degradation by contacting the RNA with a boron compound.

E2. The method according to embodiment E1 wherein the RNA is a double stranded RNA.

E3. The method according to embodiment E1 wherein the RNA is a single strand RNA.

E4. The method according to embodiment E1 wherein the RNA is an antisense single strand RNA.

E5. The method according to embodiment E1 wherein the RNA is a messenger RNA (mRNA).

E6. The method according to any one of embodiments E1 to E5 wherein the RNA is viral RNA.

E7. The method according to any one of embodiments E1 to E6 wherein the RNA is a vaccine.

E8. The method according to any one of embodiments E1 to E7 wherein the RNA is a vaccine against an arenavirus.

E9. The method according to embodiment E8 wherein the arenavirus is Lassa virus.

E10. The method according to embodiment E8 wherein the arenavirus is lymphocytic choriomeningitis virus (LCMV).

E11. The method according to any one of embodiments E1 to E6 wherein the RNA is a vaccine against an astrovirus.

E12. The method according to any one of embodiments E1 to E6 wherein the RNA is a vaccine against a bunyavirus.

E13. The method according to embodiment E12 wherein the bunyavirus is a Hantavirus.

E14. The method according to any one of embodiments E1 to E6 wherein the RNA is a vaccine against a calicivirus.

E15. The method according to any one of embodiments E1 to E6 wherein the RNA is a vaccine against a coronavirus.

E16. The method according to embodiment E15 wherein the coronavirus is a middle east respiratory syndrome (MERS) virus.

E17. The method according to embodiment E15 wherein the coronavirus is a severe acute respiratory syndrome (SARS) virus.

E18. The method according to any one of embodiments E1 to E6 wherein the RNA is a vaccine against a filovirus.

E19. The method according to embodiment E18 wherein the filovirus is Ebola virus.

E20. The method according to embodiment E18 wherein the filovirus is Marburg virus.

E21. The method according to any one of embodiments E1 to E6 wherein the RNA is a vaccine against a flavivirus.

E22. The method according to embodiment E21 wherein the flavivirus is Yellow Fever virus.

E23. The method according to embodiment E21 wherein the flavivirus is West Nile virus.

E24. The method according to embodiment E21 wherein the flavivirus is Hepatitis C virus (HCV).

E25. The method according to any one of embodiments E1 to E6 wherein the RNA is a vaccine against a hepadnavirus.

E26. The method according to any one of embodiments E1 to E6 wherein the RNA is a vaccine against a hepevirus.

E27. The method according to any one of embodiments E1 to E6 wherein the RNA is a vaccine against an orthomyxovirus.

E28. The method according to embodiment E27 wherein the orthomyxovirus is Influenza A virus.

E29. The method according to embodiment E27 wherein the orthomyxovirus is Influenza B virus.

E30. The method according to embodiment E27 wherein the orthomyxovirus is Influenza C virus.

E31. The method according to any one of embodiments E1 to E6 wherein the RNA is a vaccine against a paramyxovirus.

E32. The method according to embodiment E31 wherein the paramyxovirus is Rubeola virus.

E33. The method according to embodiment E31 wherein the paramyxovirus is Rubulavirus.

E34. The method according to any one of embodiments E1 to E6 wherein the RNA is a vaccine against a picornavirus.

E35. The method according to embodiment E34 wherein the picomvirus is Poliovirus.

E36. The method according to embodiment E34 wherein the picomvirus is Hepatitis A virus (HAV).

E37. The method according to embodiment E34 wherein the picomvirus is a Rhinovirus.

E38. The method according to any one of embodiments E1 to E6 wherein the RNA is a vaccine against a reovirus.

E39. The method according to embodiment E38 wherein the reovirus is a Rotavirus.

E40. The method according to any one of embodiments E1 to E6 wherein the RNA is a vaccine against a retrovirus.

E41. The method according to embodiment E40 wherein the retrovirus is Human Immunodeficiency Virus (HIV).

E42. The method according to embodiment E40 wherein the retrovirus is Human T-lymphotropic virus (HTLV).

E43. The method according to any one of embodiments E1 to E6 wherein the RNA is a vaccine against a rhabdovirus.

E44. The method according to embodiment E43 wherein the rhabdovirus is Rabies virus or Rabies lyssavirus.

E45. The method according to any one of embodiments E1 to E6 wherein the RNA is a vaccine against a togavirus.

E46. The method according to embodiment E45 wherein the togavirus is Sindbis virus (SINV).

E47. The method according to embodiment E45 wherein the togavirus is Eastern Equine Encephalitis virus (EEEV).

E48. The method according to embodiment E45 wherein the togavirus is Western Equine Encephalitis virus (WEEV).

E49. The method according to embodiment E45 wherein the togavirus is Rubella virus.

E50. The method according to embodiment E1 wherein the RNA has 80% to 100% sequence homology with one of the RNAs selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

E51. The method according to embodiment E1 wherein the RNA has 80% to 100% sequence homology with one of the RNAs selected from the group consisting of SEQ ID NO: 1.

E52. The method according to embodiment E1 wherein the RNA has 80% sequence homology with the RNA of SEQ ID NO: 1.

E53. The method according to embodiment E1 wherein the RNA has 90% sequence homology with the RNA of SEQ ID NO: 1.

E54. The method according to embodiment E1 wherein the RNA has 95% sequence homology with the RNA of SEQ ID NO: 1.

E55. The method according to embodiment E1 wherein the RNA has 100% sequence homology with the RNA of SEQ ID NO: 1.

E56. The method according to embodiment E1 wherein the RNA has 80% to 100% sequence homology with one of the RNAs selected from the group consisting of SEQ ID NO: 2.

E57. The method according to embodiment E1 wherein the RNA has 80% sequence homology with the RNA of SEQ ID NO: 2.

E58. The method according to embodiment E1 wherein the RNA has 90% sequence homology with the RNA of SEQ ID NO: 2.

E59. The method according to embodiment E1 wherein the RNA has 95% sequence homology with the RNA of SEQ ID NO: 2.

E60. The method according to embodiment E1 wherein the RNA has 100% sequence homology with the RNA of SEQ ID NO: 2.

E61. The method according to embodiment E1 wherein the RNA has 80% to 100% sequence homology with one of the RNAs selected from the group consisting of SEQ ID NO: 3.

E62. The method according to embodiment E1 wherein the RNA has 80% sequence homology with the RNA of SEQ ID NO: 3.

E63. The method according to embodiment E1 wherein the RNA has 90% sequence homology with the RNA of SEQ ID NO: 3.

E64. The method according to embodiment E1 wherein the RNA has 95% sequence homology with the RNA of SEQ ID NO: 3.

E65. The method according to embodiment E1 wherein the RNA has 100% sequence homology with the RNA of SEQ ID NO: 3.

E66. The method according to embodiment E1 wherein the RNA has 80% to 100% sequence homology with one of the RNAs selected from the group consisting of SEQ ID NO: 4.

E67. The method according to embodiment E1 wherein the RNA has 80% sequence homology with the RNA of SEQ ID NO: 4.

E68. The method according to embodiment E1 wherein the RNA has 90% sequence homology with the RNA of SEQ ID NO: 4.

E69. The method according to embodiment E1 wherein the RNA has 95% sequence homology with the RNA of SEQ ID NO: 4.

E70. The method according to embodiment E1 wherein the RNA has 100% sequence homology with the RNA of SEQ ID NO: 4.

E71. The method according to embodiment E1 wherein the RNA has 80% to 100% sequence homology with one of the RNAs selected from the group consisting of SEQ ID NO: 5.

E72. The method according to embodiment E1 wherein the RNA has 80% sequence homology with the RNA of SEQ ID NO: 5.

E73. The method according to embodiment E1 wherein the RNA has 90% sequence homology with the RNA of SEQ ID NO: 5.

E74. The method according to embodiment E1 wherein the RNA has 95% sequence homology with the RNA of SEQ ID NO: 5.

E75. The method according to embodiment E1 wherein the RNA has 100% sequence homology with the RNA of SEQ ID NO: 5.

E76. The method according to embodiment E1 wherein the RNA has 80% to 100% sequence homology with one of the RNAs selected from the group consisting of SEQ ID NO: 6.

E77. The method according to embodiment E1 wherein the RNA has 80% sequence homology with the RNA of SEQ ID NO: 6.

E78. The method according to embodiment E1 wherein the RNA has 90% sequence homology with the RNA of SEQ ID NO: 6.

E79. The method according to embodiment E1 wherein the RNA has 95% sequence homology with the RNA of SEQ ID NO: 6.

E80. The method according to embodiment E1 wherein the RNA has 100% sequence homology with the RNA of SEQ ID NO: 6.

E81. The method according to embodiment E1 wherein the RNA has 80% to 100% sequence homology with one of the RNAs selected from the group consisting of SEQ ID NO: 7.

E82. The method according to embodiment E1 wherein the RNA has 80% sequence homology with the RNA of SEQ ID NO: 7.

E83. The method according to embodiment E1 wherein the RNA has 90% sequence homology with the RNA of SEQ ID NO: 7.

E84. The method according to embodiment E1 wherein the RNA has 95% sequence homology with the RNA of SEQ ID NO: 7.

E85. The method according to embodiment E1 wherein the RNA has 100% sequence homology with the RNA of SEQ ID NO: 7.

E86. The method according to embodiment E1 wherein the RNA has 80% to 100% sequence homology with one of the RNAs selected from the group consisting of SEQ ID NO: 8.

E87. The method according to embodiment E1 wherein the RNA has 80% sequence homology with the RNA of SEQ ID NO: 8.

E88. The method according to embodiment E1 wherein the RNA has 90% sequence homology with the RNA of SEQ ID NO: 8.

E89. The method according to embodiment E1 wherein the RNA has 95% sequence homology with the RNA of SEQ ID NO: 8.

E90. The method according to embodiment E1 wherein the RNA has 100% sequence homology with the RNA of SEQ ID NO: 8.

E91. The method according to any one of embodiments E1 to E90 wherein the RNA is contained within a non-viral vector.

E92. The method according to any one of embodiments E1 to E90 wherein the RNA is contained within a nanoparticle.

E93. The method according to any one of embodiments E1 to E90 wherein the RNA is contained within a liposome.

E94. The method according to any one of embodiments E1 to E90 wherein the RNA is contained within a micelle.

E95. The method according to any one of embodiments E1 to E90 wherein the RNA is contained within a viral vector.

E96. The method according to any one of embodiments E1 to E90 wherein the RNA is contained within an Adenovirus (Ad).

E97. The method according to any one of embodiments E1 to E90 wherein the RNA is contained within an adeno-associated virus (AAV).

E98. The method according to any one of embodiments E1 to E90 wherein the RNA is contained within a retrovirus.

E99. The method according to any one of embodiments E1 to E90 wherein the RNA is contained within a lentivirus.

E100. The method according to any one of embodiments E1 to E99 wherein the boron compound is N-(1-hydroxy-1,3-dihydrobenzo[c][1,2]oxaborol-6-yl)benzamide (PF-06957640) or a pharmaceutically acceptable salt or deuterated analogue thereof.

E101. The method according to any one of embodiments E1 to E99 wherein the boron compound is 2,2′-(cyclohexane-1,1-diyl)bis(4,4,5,5-tetramethyl-1,3,2-dioxaborolane) (PF-07230998) or a pharmaceutically acceptable salt or deuterated analogue thereof.

E102. The method according to any one of embodiments E1 to E99 wherein the boron compound is 5-(2-(aminomethyl)-4-(trifluoromethyl)phenoxy)benzo[c][1,2]oxaborol-1(3H)-ol (PF-06967658) or a pharmaceutically acceptable salt or deuterated analogue thereof.

E103. The method according to any one of embodiments E1 to E99 wherein the boron compound is 1-hydroxy-3,3-dimethyl-1,3-dihydrobenzo[c][1,2]oxaborole-6-carboxylic acid (PF-06966741) or a pharmaceutically acceptable salt or deuterated analogue thereof.

E104. The method according to any one of embodiments E1 to E99 wherein the boron compound is N-(3-(1-hydroxy-1,3-dihydrobenzo[c][1,2]oxaborol-4-yl)phenyl)-5-(1-methyl-1H-benzo[d]imidazol-2-yl)pentanamide (PF-06963078) or a pharmaceutically acceptable salt or deuterated analogue thereof.

E105. The method according to any one of embodiments E1 to E99 wherein the boron compound is 4-(5-(3-chlorophenyl)-5-(trifluoromethyl)-4,5-dihydroisoxazol-3-yl)-N-(1-hydroxy-2-methyl-1,2-dihydrobenzo[d][1,2,3]diazaborinin-6-yl)-2-methylbenzamide (PF-06962739) or a pharmaceutically acceptable salt or deuterated analogue thereof.

E106. The method according to any one of embodiments E1 to E99 wherein the boron compound is 3-(4-fluorobenzyl)-5-(4-(((1-hydroxy-1,3-dihydrobenzo[c][1,2]oxaborol-6-yl)oxy)methyl)benzylidene)thiazolidine-2,4-dione (PF-06963029) or a pharmaceutically acceptable salt or deuterated analogue thereof.

E107. The method according to any one of embodiments E1 to E99 wherein the boron compound is 4-(5-(3,5-dichloro-4-fluorophenyl)-5-(trifluoromethyl)-4,5-dihydroisoxazol-3-yl)-N-((1-hydroxy-3,3-dimethyl-1,3-dihydrobenzo[c][1,2]oxaborol-5-yl)methyl)-2-methylbenzamide (PF-06962691) or a pharmaceutically acceptable salt or deuterated analogue thereof.

E108. The method according to any one of embodiments E1 to E99 wherein the boron compound is 2-(6-((2-((2-aminoethyl)amino)pyrimidin-4-yl)oxy)-1-hydroxy-4-methyl-1,3-dihydrobenzo[c][1,2]oxaborol-3-yl)acetic acid (PF-06959336) or a pharmaceutically acceptable salt or deuterated analogue thereof.

E109. The method according to any one of embodiments E1 to E99 wherein the boron compound is 5-phenoxybenzo[c][1,2]oxaborol-1(3H)-ol (PF-01499456) or a pharmaceutically acceptable salt or deuterated analogue thereof.

E110. The method according to any one of embodiments E1 to E99 wherein the boron compound is 5-(pyridin-2-ylmethoxy)benzo[c][1,2]oxaborol-1(3H)-ol (PF-06957808) or a pharmaceutically acceptable salt or deuterated analogue thereof.

E111. The method according to any one of embodiments E1 to E99 wherein the boron compound is 8-(3-aminopropoxy)benzo[d][1,2,3]diazaborinin-1(2H)-ol (PF-06957880) or a pharmaceutically acceptable salt or deuterated analogue thereof.

E112. The method according to any one of embodiments E1 to E99 wherein the boron compound is 4-(aminomethyl)-N-(1-hydroxy-1,3-dihydrobenzo[c][1,2]oxaborol-6-yl)benzenesulfonamide (PF-06957807) or a pharmaceutically acceptable salt or deuterated analogue thereof.

E113. The method according to any one of embodiments E1 to E99 wherein the boron compound is N-((1R,2R)-1-(4-((S)-3-(aminomethyl)-1-hydroxy-1,3-dihydrobenzo[c][1,2]oxaborol-6-yl)phenyl)-1,3-dihydroxypropan-2-yl)-2-chloroacetamide (PF-06969336) or a pharmaceutically acceptable salt or deuterated analogue thereof.

E114. The method according to any one of embodiments E1 to E99 wherein the boron compound is disclosed in WO2004/056322, or a pharmaceutically acceptable salt or deuterated analogue thereof.

E115. The method according to any one of embodiments E1 to E99 wherein the boron compound is disclosed in WO2005/013892, or a pharmaceutically acceptable salt or deuterated analogue thereof.

E116. The method according to any one of embodiments E1 to E99 wherein the boron compound is disclosed in WO2005/123094, or a pharmaceutically acceptable salt or deuterated analogue thereof.

E117. The method according to any one of embodiments E1 to E99 wherein the boron compound is disclosed in WO2006/007384, or a pharmaceutically acceptable salt or deuterated analogue thereof.

E118. The method according to any one of embodiments E1 to E99 wherein the boron compound is disclosed in WO2006/007384, or a pharmaceutically acceptable salt or deuterated analogue thereof.

E119. The method according to any one of embodiments E1 to E99 wherein the boron compound is disclosed in WO2006/085932, or a pharmaceutically acceptable salt or deuterated analogue thereof.

E120. The method according to any one of embodiments E1 to E99 wherein the boron compound is disclosed in WO2006/089067, or a pharmaceutically acceptable salt or deuterated analogue thereof.

E121. The method according to any one of embodiments E1 to E99 wherein the boron compound is disclosed in WO2007/078340, or a pharmaceutically acceptable salt or deuterated analogue thereof.

E122. The method according to any one of embodiments E1 to E99 wherein the boron compound is disclosed in WO2007/095638, or a pharmaceutically acceptable salt or deuterated analogue thereof.

E123. The method according to any one of embodiments E1 to E99 wherein the boron compound is disclosed in WO2007/131072, or a pharmaceutically acceptable salt or deuterated analogue thereof.

E124. The method according to any one of embodiments E1 to E99 wherein the boron compound is disclosed in WO2007/146965, or a pharmaceutically acceptable salt or deuterated analogue thereof.

E125. The method according to any one of embodiments E1 to E99 wherein the boron compound is disclosed in WO2007/079119, or a pharmaceutically acceptable salt or deuterated analogue thereof.

E126. The method according to any one of embodiments E1 to E99 wherein the boron compound is disclosed in WO2008/157726, or a pharmaceutically acceptable salt or deuterated analogue thereof.

E127. The method according to any one of embodiments E1 to E99 wherein the boron compound is disclosed in WO2009/111676, or a pharmaceutically acceptable salt or deuterated analogue thereof.

E128. The method according to any one of embodiments E1 to E99 wherein the boron compound is disclosed in WO2009/140309, or a pharmaceutically acceptable salt or deuterated analogue thereof.

E129. The method according to any one of embodiments E1 to E99 wherein the boron compound is disclosed in WO2010/028005, or a pharmaceutically acceptable salt or deuterated analogue thereof.

E130. The method according to any one of embodiments E1 to E99 wherein the boron compound is disclosed in WO2010/027975, or a pharmaceutically acceptable salt or deuterated analogue thereof.

E131. The method according to any one of embodiments E1 to E99 wherein the boron compound is disclosed in WO2010/045503, or a pharmaceutically acceptable salt or deuterated analogue thereof.

E132. The method according to any one of embodiments E1 to E99 wherein the boron compound is disclosed in WO2010/045505, or a pharmaceutically acceptable salt or deuterated analogue thereof.

E133. The method according to any one of embodiments E1 to E99 wherein the boron compound is disclosed in WO2010/080558, or a pharmaceutically acceptable salt or deuterated analogue thereof.

E134. The method according to any one of embodiments E1 to E99 wherein the boron compound is disclosed in WO2011/049971, or a pharmaceutically acceptable salt or deuterated analogue thereof.

E135. The method according to any one of embodiments E1 to E99 wherein the boron compound is disclosed in WO2011/017125, or a pharmaceutically acceptable salt or deuterated analogue thereof.

E136. The method according to any one of embodiments E1 to E99 wherein the boron compound is disclosed in WO2011/019618, or a pharmaceutically acceptable salt or deuterated analogue thereof.

E137. The method according to any one of embodiments E1 to E99 wherein the boron compound is disclosed in WO2011/022337, or a pharmaceutically acceptable salt or deuterated analogue thereof.

E138. The method according to any one of embodiments E1 to E99 wherein the boron compound is disclosed in WO2011/037731, or a pharmaceutically acceptable salt or deuterated analogue thereof.

E139. The method according to any one of embodiments E1 to E99 wherein the boron compound is disclosed in WO2011/060196, or a pharmaceutically acceptable salt or deuterated analogue thereof.

E140. The method according to any one of embodiments E1 to E99 wherein the boron compound is disclosed in WO2011/060199, or a pharmaceutically acceptable salt or deuterated analogue thereof.

E141. The method according to any one of embodiments E1 to E99 wherein the boron compound is disclosed in WO2011/094450, or a pharmaceutically acceptable salt or deuterated analogue thereof.

E142. The method according to any one of embodiments E1 to E99 wherein the boron compound is disclosed in WO2011/116348, or a pharmaceutically acceptable salt or deuterated analogue thereof.

E143. The method according to any one of embodiments E1 to E99 wherein the boron compound is disclosed in WO2012/033858, or a pharmaceutically acceptable salt or deuterated analogue thereof.

E144. The method according to any one of embodiments E1 to E99 wherein the boron compound is disclosed in WO2013/078070, or a pharmaceutically acceptable salt or deuterated analogue thereof.

E145. The method according to any one of embodiments E1 to E99 wherein the boron compound is disclosed in WO2013/078071, or a pharmaceutically acceptable salt or deuterated analogue thereof.

E146. The method according to any one of embodiments E1 to E99 wherein the boron compound is disclosed in WO2014/121124, or a pharmaceutically acceptable salt or deuterated analogue thereof.

E147. The method according to any one of embodiments E1 to E99 wherein the boron compound is disclosed in WO2014/149793, or a pharmaceutically acceptable salt or deuterated analogue thereof.

E148. The method according to any one of embodiments E1 to E99 wherein the boron compound is disclosed in WO2015/013318, or a pharmaceutically acceptable salt or deuterated analogue thereof.

E149. The method according to any one of embodiments E1 to E99 wherein the boron compound is disclosed in WO2015/042532, or a pharmaceutically acceptable salt or deuterated analogue thereof.

E150. The method according to any one of embodiments E1 to E99 wherein the boron compound is disclosed in WO2018/160845, or a pharmaceutically acceptable salt or deuterated analogue thereof.

E151. The method according to any one of embodiments E1 to E99 wherein the boron compound is disclosed in WO2020/070651, or a pharmaceutically acceptable salt or deuterated analogue thereof.

E152. The method according to any one of embodiments E1 to E99 wherein the boron compound is disclosed in US7465836, or a pharmaceutically acceptable salt or deuterated analogue thereof.

E153. The method according to any one of embodiments E1 to E99 wherein the boron compound is disclosed in US8106031, or a pharmaceutically acceptable salt or deuterated analogue thereof.

E154. The method according to any one of embodiments E1 to E99 wherein the boron compound is disclosed in US8623911, or a pharmaceutically acceptable salt or deuterated analogue thereof.

E155. The method according to any one of embodiments E1 to E99 wherein the boron compound is disclosed in US9346834, or a pharmaceutically acceptable salt or deuterated analogue thereof.

E156. The method according to any one of embodiments E1 to E155 wherein the boron compound has a protective effect in the RNA Fluorescence Polarization (FP) Protection Assay.

E157. The method according to embodiment E156 wherein the boron compound produces percent activity in the FP Protection Assay.

E158. The method according to embodiment E157 wherein the boron compound produces at least 10% activity in the RNA FP Protection Assay.

E159. The method according to embodiment E157 wherein the boron compound produces at least 20% activity in the RNA FP Protection Assay.

E160. The method according to embodiment E157 wherein the boron compound produces at least 30% activity in the RNA FP Protection Assay.

E161. The method according to embodiment E157 wherein the boron compound produces at least 40% activity in the RNA FP Protection Assay.

E162. The method according to embodiment E157 wherein the boron compound produces at least 50% activity in the RNA FP Protection Assay.

E163. The method according to embodiment E157 wherein the boron compound produces at least 60% activity in the RNA FP Protection Assay.

E164. The method according to embodiment E157 wherein the boron compound produces at least 70% activity in the RNA FP Protection Assay.

E165. The method according to embodiment E157 wherein the boron compound produces at least 80% activity in the RNA FP Protection Assay.

E166. The method according to embodiment E157 wherein the boron compound produces at least 90% activity in the RNA FP Protection Assay.

E167. The method according to any one of embodiments E156 to E166 wherein the percent activity in the RNA FP Protection Assay is due to reduced hydrolysis of the RNA.

E168. The method according to embodiment E156 wherein the boron compound produces an increase in the mili-Polarization (mP) value in a FP measurement.

E169. The method according to embodiment E168 wherein the increase in mP in the RNA FP Protection Assay is due to reduced hydrolysis of the RNA.

E170. The method according to any one of embodiments E156 to E169 wherein the percent activity or mP value in the RNA FP Protection Assay is measured under conditions that accelerate RNA degradation.

E171. The method according to any one of embodiments E156 to E170 wherein the percent activity or mP value is measured under basic conditions after 18 hours.

E172. The method according to any one of embodiments E1 to E171 wherein the boron compound has a protective effect in the syto-9 dye intercalation assay.

E173. The method according to embodiment E172 wherein the boron compound produces a signal in the syto-9 dye intercalation assay.

E174. The method according to embodiment E173 wherein the boron compound has an EC₅₀ equal to or less than 300 µM in the syto-9 dye intercalation assay.

E175. The method according to embodiment E173 wherein the boron compound has an EC₅₀ equal to or less than 200 µM in the syto-9 dye intercalation assay.

E176. The method according to embodiment E173 wherein the boron compound has an EC₅₀ equal to or less than 100 µM in the syto-9 dye intercalation assay.

E177. The method according to embodiment E173 wherein the boron compound has an EC₅₀ equal to or less than 50 µM in the syto-9 dye intercalation assay.

E178. The method according to embodiment E173 wherein the boron compound has an EC₅₀ equal to or less than 10 µM in the syto-9 dye intercalation assay.

E179. The method according to any one of embodiments E172 to E178 wherein the boron compound produces no signal in the counter screen syto-9 dye intercalation assay.

E180. The method according to any one of embodiments E172 to E179 wherein the boron compound’s EC₅₀ in the counter screen syto-9 dye intercalation assay is greater than 300 µM.

E181. The method according to any one of embodiments E172 to E179 wherein the boron compound’s EC₅₀ in the counter screen syto-9 dye intercalation assay is greater than 400 µM.

E182. The method according to any one of embodiments E172 to E179 wherein the boron compound’s EC₅₀ in the counter screen syto-9 dye intercalation assay is greater than 500 µM.

E183. The method according to any one of embodiments E172 to E182 wherein the EC₅₀ in the syto-9 dye intercalation assay and the EC₅₀ in the counter screen syto-9 dye intercalation assay are measured under conditions that accelerate RNA degradation.

E184. The method according to any one of embodiments E172 to E183 wherein the EC₅₀ in the syto-9 dye intercalation assay and the EC₅₀ in the counter screen syto-9 dye intercalation assay are measured under basic conditions after 18 hours.

E185. The method according to any one of embodiments E1 to E184 wherein the boron compound has a protective effect in the Droplet Digital Polymerase Chain Reaction (ddPCR) Assay.

E186. The method according to any one of embodiments E1 to E185 wherein the boron compound produces a measurable percent protection in the Droplet Digital Polymerase Chain Reaction (ddPCR) Assay.

E187. The method according to embodiment E186 wherein the boron compound produces a percent protection of at least 10% in the ddPCR assay.

E188. The method according to embodiment E186 wherein the boron compound produces a percent protection of at least 20% in the ddPCR assay.

E189. The method according to embodiment E186 wherein the boron compound produces a percent protection of at least 30% in the ddPCR assay.

E190. The method according to embodiment E186 wherein the boron compound produces a percent protection of at least 40% in the ddPCR assay.

E191. The method according to embodiment E186 wherein the boron compound produces a percent protection of at least 50% in the ddPCR assay.

E192. The method according to embodiment E186 wherein the boron compound produces a percent protection of at least 60% in the ddPCR assay.

E193. The method according to embodiment E186 wherein the boron compound produces a percent protection of at least 70% in the ddPCR assay.

E194. The method according to embodiment E186 wherein the boron compound produces a percent protection of at least 80% in the ddPCR assay.

E195. The method according to embodiment E186 wherein the boron compound produces a percent protection of at least 90% in the ddPCR assay.

E196. The method according to any one of embodiments E1 to E195 wherein the boron compound has an EC₅₀ equal to or less than 700 µM in the ddPCR assay.

E197. The method according to any one of embodiments E1 to E195 wherein the boron compound has an EC₅₀ equal to or less than 600 µM in the ddPCR assay.

E198. The method according to any one of embodiments E1 to E195 wherein the boron compound has an EC₅₀ equal to or less than 500 µM in the ddPCR assay.

E199. The method according to any one of embodiments E1 to E195 wherein the boron compound has an EC₅₀ equal to or less than 400 µM in the ddPCR assay.

E200. The method according to any one of embodiments E1 to E195 wherein the boron compound has an EC₅₀ equal to or less than 300 µM in the ddPCR assay.

E201. The method according to any one of embodiments E1 to E195 wherein the boron compound has an EC₅₀ equal to or less than 200 µM in the ddPCR assay.

E202. The method according to any one of embodiments E1 to E195 wherein the boron compound has an EC₅₀ equal to or less than 100 µM in the ddPCR assay.

E203. The method according to any one of embodiments E1 to E195 wherein the boron compound has an EC₅₀ equal to or less than 50 µM in the ddPCR assay.

E204. The method according to any one of embodiments E186 to E203 wherein the percent protection is measured in the ddPCR assay under conditions that accelerate RNA degradation.

E205. The method according to any one of embodiments E186 to E203 wherein the percent protection is measured in the ddPCR assay under conditions pH 11.4, 30° C., and 16 hours.

E206. The method according to any one of embodiments E186 to E203 wherein the percent protection is measured in the ddPCR assay under conditions pH 11.4, 42° C., and 16 hours.

E207. The method according to any one of embodiments E186 to E203 wherein the boron compound’s EC₅₀ is measured in the ddPCR assay under conditions that accelerate RNA degradation.

E208. The method according to any one of embodiments E186 to E203 wherein the boron compound’s EC₅₀ is measured under conditions pH 11.4, 30° C., and 16 hours in the ddPCR assay.

E209. The method according to any one of embodiments E186 to E203 wherein the boron compound’s EC₅₀ is measured under conditions pH 11.4, 42° C., and 16 hours in the ddPCR assay.

E210. The method according to any one of embodiments E1 to E209 wherein the boron compound has a protective effect in the TapeStation Assay.

E211. The method according to embodiment 210 wherein the boron compound protects at least 10% of the RNA in the TapeStation Assay.

E212. The method according to embodiment 210 wherein the boron compound protects at least 20% of the RNA in the TapeStation Assay.

E213. The method according to embodiment 210 wherein the boron compound protects at least 30% of the RNA in the TapeStation Assay.

E214. The method according to embodiment 210 wherein the boron compound protects at least 40% of the RNA in the TapeStation Assay.

E215. The method according to embodiment 210 wherein the boron compound protects at least 50% of the RNA in the TapeStation Assay.

E216. The method according to embodiment 210 wherein the boron compound protects at least 60% of the RNA in the TapeStation Assay.

E217. The method according to embodiment 210 wherein the boron compound protects at least 70% of the RNA in the TapeStation Assay.

E218. The method according to embodiment 210 wherein the boron compound protects at least 80% of the RNA in the TapeStation Assay.

E219. The method according to embodiment 210 wherein the boron compound protects at least 90% of the RNA in the TapeStation Assay.

E220. The method according to embodiment 210 wherein the boron compound protects 100% of the RNA in the TapeStation Assay.

E221. The method according to any one of embodiments E210 to E220 wherein percentage of RNA protected is measured in the TapeStation Assay under conditions that accelerate RNA degradation.

E222. The method according to any one of embodiments E210 to E220 wherein percentage of RNA protected is measured in the TapeStation Assay under conditions pH 11.4, 78° C., and 8 minutes.

E223. The method according to any one of embodiments E210 to E220 wherein percentage of RNA protected is measured in the TapeStation Assay under conditions pH 11.4, 65° C., and 5 minutes.

E224. The method according to any one of embodiments E210 to E220 wherein percentage of RNA protected is measured in the TapeStation Assay under conditions pH 11.4, 65° C., and 10 minutes.

E225. A composition comprising an RNA and a boron compound.

E226. The composition according to embodiment E225 wherein the RNA is a double stranded RNA.

E227. The composition according to embodiment E225 wherein the RNA is a single strand RNA.

E228. The composition according to embodiment E225 wherein the RNA is an antisense single strand RNA.

E229. The composition according to embodiment E225 wherein the RNA is a messenger RNA (mRNA).

E230. The composition according to embodiment E225 wherein the RNA is a messenger RNA (mRNA).

E231. The composition according to any one of embodiments E225 to E230 wherein the RNA is viral RNA.

E232. The composition according to any one of embodiments E225 to E231 wherein the RNA is a vaccine.

E233. The composition according to any one of embodiments E225 to E232 wherein the RNA is a vaccine against an arenavirus.

E234. The composition according to embodiment E233 wherein the arenavirus is Lassa virus.

E235. The composition according to embodiment E233 wherein the arenavirus is lymphocytic choriomeningitis virus (LCMV).

E236. The composition according to any one of embodiments E225 to E232 wherein the RNA is a vaccine against an astrovirus.

E237. The composition according to any one of embodiments E225 to E232 wherein the RNA is a vaccine against a bunyavirus.

E238. The composition according to embodiment E237 wherein the bunyavirus is a Hantavirus.

E239. The composition according to any one of embodiments E225 to E232 wherein the RNA is a vaccine against a calicivirus.

E240. The composition according to any one of embodiments E225 to E232 wherein the RNA is a vaccine against a coronavirus.

E241. The composition according to embodiment E240 wherein the coronavirus is a middle east respiratory syndrome (MERS) virus.

E242. The composition according to embodiment E240 wherein the coronavirus is a severe acute respiratory syndrome (SARS) virus.

E243. The composition according to any one of embodiments E225 to E232 wherein the RNA is a vaccine against a filovirus.

E244. The composition according to embodiment E243 wherein the filovirus is Ebola virus.

E245. The composition according to embodiment E243 wherein the filovirus is Marburg virus.

E246. The composition according to any one of embodiments E225 to E232 wherein the RNA is a vaccine against a flavivirus.

E247. The composition according to embodiment E246 wherein the flavivirus is Yellow Fever virus.

E248. The composition according to embodiment E246 wherein the flavivirus is West Nile virus.

E249. The composition according to embodiment E246 wherein the flavivirus is Hepatitis C virus (HCV).

E250. The composition according to any one of embodiments E225 to E232 wherein the RNA is a vaccine against a hepadnavirus.

E251. The composition according to any one of embodiments E225 to E232 wherein the RNA is a vaccine against a hepevirus.

E252. The composition according to any one of embodiments E225 to E232 wherein the RNA is a vaccine against an orthomyxovirus.

E253. The composition according to embodiment E252 wherein the orthomyxovirus is Influenza A virus.

E254. The composition according to embodiment E252 wherein the orthomyxovirus is Influenza B virus.

E255. The composition according to embodiment E252 wherein the orthomyxovirus is Influenza C virus.

E256. The composition according to any one of embodiments E225 to E232 wherein the RNA is a vaccine against a paramyxovirus.

E257. The composition according to embodiment E256 wherein the paramyxovirus is Rubeola virus.

E258. The composition according to embodiment E256 wherein the paramyxovirus is Rubulavirus.

E259. The composition according to any one of embodiments E225 to E232 wherein the RNA is a vaccine against a picornavirus.

E260. The composition according to embodiment E259 wherein the picomvirus is Poliovirus.

E261. The composition according to embodiment E259 wherein the picomvirus is Hepatitis A virus (HAV).

E262. The composition according to embodiment E259 wherein the picomvirus is a Rhinovirus.

E263. The composition according to any one of embodiments E225 to E232 wherein the RNA is a vaccine against a reovirus.

E264. The composition according to embodiment E263 wherein the reovirus is a Rotavirus.

E265. The composition according to any one of embodiments E225 to E232 wherein the RNA is a vaccine against a retrovirus.

E266. The composition according to embodiment E265 wherein the retrovirus is Human Immunodeficiency Virus (HIV).

E267. The composition according to embodiment E265 wherein the retrovirus is Human T-lymphotropic virus (HTLV).

E268. The composition according to any one of embodiments E225 to E232 wherein the RNA is a vaccine against a rhabdovirus.

E269. The composition according to embodiment E268 wherein the rhabdovirus is Rabies virus or Rabies lyssavirus.

E270. The composition according to any one of embodiments E225 to E232 wherein the RNA is a vaccine against a togavirus.

E271. The composition according to embodiment E270 wherein the togavirus is Sindbis virus (SINV).

E272. The composition according to embodiment E270 wherein the togavirus is Eastern Equine Encephalitis virus (EEEV).

E273. The composition according to embodiment E270 wherein the togavirus is Western Equine Encephalitis virus (WEEV).

E274. The composition according to embodiment E270 wherein the togavirus is Rubella virus.

E275. The composition according to embodiment E225 wherein the RNA is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9.

E276. The composition according to any one of embodiments E225 to E275 wherein the boron compound is disclosed in WO2004/056322, WO2005/013892, WO2005/123094, WO2006/007384, WO2006/007384, WO2006/085932, WO2006/089067, WO2007/078340, WO2007/095638, WO2007/131072, WO2007/146965, WO2007/079119, WO2008/157726, WO2009/111676, WO2009/140309, WO2010/028005, WO2010/027975, WO2010/045503, WO2010/045505, WO2010/080558, WO2011/049971, WO2011/017125, WO2011/019618, WO2011/022337, WO2011/037731, WO2011/060196, WO2011/060199, WO2011/094450, WO2011/116348, WO2012/033858, WO2013/078070, WO2013/078071, WO2014/121124, WO2014/149793, WO2015/013318, WO2015/042532, WO2018/160845, WO2020/070651, US7465836, US8106031, US8623911, or US9346834.

E277. The composition according to any one of embodiments E225 to E275 wherein the boron compound is selected from the group consisting of N-(1-hydroxy-1,3-dihydrobenzo[c][1,2]oxaborol-6-yl)benzamide; 2,2′-(cyclohexane-1,1-diyl)bis(4,4,5,5-tetramethyl-1,3,2-dioxaborolane); 5-(2-(aminomethyl)-4-(trifluoromethyl)phenoxy)benzo[c][1,2]oxaborol-1(3H)-ol; 1-hydroxy-3,3-dimethyl-1,3-dihydrobenzo[c][1,2]oxaborole-6-carboxylic acid; N-(3-(1-hydroxy-1,3-dihydrobenzo[c][1,2]oxaborol-4-yl)phenyl)-5-(1-methyl-1H- benzo[d]imidazol-2-yl)pentanamide; 4-(5-(3-chlorophenyl)-5-(trifluoromethyl)-4,5-dihydroisoxazol-3-yl)-N-(1-hydroxy-2-methyl-1,2-dihydrobenzo[d][1,2,3]diazaborinin-6-yl)-2-methylbenzamide; 3-(4-fluorobenzyl)-5-(4-(((1-hydroxy-1,3-dihydrobenzo[c][1,2]oxaborol-6-yl)oxy)methyl)benzylidene)thiazolidine-2,4-dione; 4-(5-(3,5-dichloro-4-fluorophenyl)-5-(trifluoromethyl)-4,5-dihydroisoxazol-3-yl)-N-((1-hydroxy-3,3-dimethyl-1,3-dihydrobenzo[c][1,2]oxaborol-5-yl)methyl)-2-methylbenzamide; 2-(6-((2-((2-aminoethyl)amino)pyrimidin-4-yl)oxy)-1-hydroxy-4-methyl-1,3-dihydrobenzo[c][1,2]oxaborol-3-yl)acetic acid; 5-phenoxybenzo[c][1,2]oxaborol-1(3H)-ol; 5-(pyridin-2-ylmethoxy)benzo[c][1,2]oxaborol-1(3H)-ol; 8-(3-aminopropoxy)benzo[d][1,2,3]diazaborinin-1(2H)-ol; 4-(aminomethyl)-N-(1-hydroxy-1,3-dihydrobenzo[c][1,2]oxaborol-6-yl)benzenesulfonamide; and N-((1R,2R)-1-(4-((S)-3-(aminomethyl)-1-hydroxy-1,3-dihydrobenzo[c][1,2]oxaborol-6-yl)phenyl)-1,3-dihydroxypropan-2-yl)-2-chloroacetamide; or a pharmaceutically acceptable salt or deuterated analogue thereof.

E278. The composition according to any one of embodiments E225 to E275 wherein the boron compound is 2-(6-((2-((2-aminoethyl)amino)pyrimidin-4-yl)oxy)-1-hydroxy-4-methyl-1,3-dihydrobenzo[c][1,2]oxaborol-3-yl)acetic acid or a pharmaceutically acceptable salt or deuterated analogue thereof.

E279. The composition according to any one of embodiments E225 to E278 wherein the RNA and boron compound are contained within a non-viral vector.

E280. The composition according to any one of embodiments E225 to E278 wherein the RNA and boron compound are contained within a nanoparticle.

E281. The composition according to any one of embodiments E225 to E278 wherein the RNA and boron compound are contained within a liposome.

E282. The composition according to any one of embodiments E225 to E278 wherein the RNA and boron compound are contained within a micelle.

E283. The composition according to any one of embodiments E225 to E278 wherein the RNA and boron compound are contained within a viral vector.

E284. The composition according to any one of embodiments E225 to E278 wherein the RNA and boron compound are contained within an Adenovirus.

E285. The composition according to any one of embodiments E225 to E278 wherein the RNA and boron compound are contained within an adeno-associated virus.

E286. The composition according to any one of embodiments E225 to E278 wherein the RNA and boron compound are contained within a retrovirus.

E287. The composition according to any one of embodiments E225 to E278 wherein the RNA and boron compound are contained within a lentivirus.

E288. The method according to any one of embodiments E1 to E99 wherein the boron compound is N-[(5aR,6aS,78,10aS)-9-carbamoyl-4,7-bis(dimethylamino)-1,8,10a,11-tetrahydroxy-10,12-dioxo-5,5a,6,6a,7,10,10a,12-octahydrotetracen-2-yl]-1-hydroxy-3,3-dimethyl-1,3-dihydro-2,1-benzoxaborole-6-carboxamide (PF-06967791) or a pharmaceutically acceptable salt or deuterated analogue thereof.

E289. The method according to any one of embodiments E1 to E99 wherein the boron compound is 4S,4aR,5S,5aR,6R,12aS)-4-(dimethylamino)-3,5,10,12,12a-pentahydroxy-9-({2-[(1-hydroxy-1,3-dihydro-2,1-benzoxaborob6-yl)amino]-2-oxoethyl}amino)-6-methyl-1,11-dioxo-1,4,4a,5,5a,6,11,12a-octahydrotetracene-2-carboxamide (PF-06968419) or a pharmaceutically acceptable salt or deuterated analogue thereof.

E290. The method according to any one of embodiments E1 to E99 wherein the boron compound is N~1~-[(5R,5aR,6S,6aR,7S,10aS)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-N-4~-(1-hydroxy-1,3-dihydro-2,1-benzoxaborol-6-yl)butanediamide (PF-06968256) or a pharmaceutically acceptable salt or deuterated analogue thereof.

E291. The method according to any one of embodiments E1 to E99 wherein the boron compound is (4S,4aR,5S,5aR,6R,12aS)-4-(dimethylamino)-3,5,10,12,12a-pentahydroxy-9-({3-[(1-hydroxy-3,4-dihydro-1H-2,5,1-benzodioxaborepin-7-yl)amino]-3-oxopropyl}amino)-6-methyl-1,11-dioxo-1,4,4a,5,5a,6,11,12a-oc tahydrotetracene-2-carboxamide (PF-06968559) or a pharmaceutically acceptable salt or deuterated analogue thereof.

E292. The method according to any one of embodiments E1 to E99 wherein the boron compound is [(3S)-6-({2-[(2-aminoethyl)amino]pyrimidin-4-yl}oxy)-1-hydroxy-4-methyl-1,3-dihydro-2,1-benzoxaborol-3-yl]acetic acid (PF-06959336) or a pharmaceutically acceptable salt or deuterated analogue thereof.

E293. The method according to any one of embodiments E1 to E99 wherein the boron compound is (4S,4aR,5S,5aR,6R,12aS)-9-({3-[(2,3-dihydro-7H-[1,2]oxaborolo[4,3,2-jk][2,5,1]benzodioxaborepin-9-yl)amino]-3-oxopropyl}amino)-4-(dimethylamino)-3,5,10,12,12a-pentahydroxy-6-methyl-1,11-dioxo-1,4,4a,5,5a,6,11,12a-octahydrotetracene-2-carboxamide (PF-06968542) or a pharmaceutically acceptable salt or deuterated analogue thereof.

E294. The method according to any one of embodiments E1 to E99 wherein the boron compound is quinolin-8-yl {3-chloro-4-[(dimethylamino)methyl]phenyl}(3-cyanophenyl)borinate (PF-06956816) or a pharmaceutically acceptable salt or deuterated analogue thereof.

E295. The method according to any one of embodiments E1 to E99 wherein the boron compound is (4-fluorophenyl){3-[(4-methylpiperazin-1-yl)methyl]phenyl}(quinolin-8-olato-kappa~2~N,O)boron (PF-06956810) or a pharmaceutically acceptable salt or deuterated analogue thereof.

E296. The method according to any one of embodiments E1 to E99 wherein the boron compound is N-[(5R,5aR,6S,6aR,7S,10aS)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-1-hydroxy-3,3-dimethyl-1,3-dihydro-2, 1-benzoxaboro le-6-carboxamide (PF-06968101) or a pharmaceutically acceptable salt or deuterated analogue thereof.

E297. The method according to any one of embodiments E1 to E99 wherein the boron compound is (4S,4aR,5S,5aR,6R,12aS)-4-(dimethylamino)-3,5,10,12,12a-pentahydroxy-9-({3-[(1-hydroxy-3,4-dihydro-1H-2,1-benzoxaborinin-7-yl)amino]-3-oxopropyl}amino)-6-methyl-1,11-dioxo-1,4,4a,5,5a,6,11,12a-octahyd rotetracene-2-carboxamide (PF-06968237) or a pharmaceutically acceptable salt or deuterated analogue thereof.

E298. The method according to any one of embodiments E1 to E99 wherein the boron compound is quinolin-8-yl (3-chloro-4-cyanophenyl){3-fluoro-4-[(4-methylpiperazin-1-yl)methyl]phenyl}borinate (PF-06956823) or a pharmaceutically acceptable salt or deuterated analogue thereof.

E299. The method according to any one of embodiments E1 to E99 wherein the boron compound is N~1~-[(5RS,5aRS6SR,6aRS,7SR,10aSR)-9-carbamoyl-7-(dimethylamino)-1,6,8,10a,11-pentahydroxy-5-methyl-10, 12-dioxo-5,5a,6,6a,7,10,10a,12-octahydrotetracen-2-yl}-N~4~-{[(5RS)-2-hydroxy-1,2-oxaborolan-5-yl]methyl}butanediamide (PF-06968488) or a pharmaceutically acceptable salt or deuterated analogue thereof.

E300. The method according to any one of embodiments E1 to E99 wherein the boron compound is {3-chloro-4-[(4-methylpiperazin-1-yl)methyl]phenyl}(4-cyanophenyl)(quinolin-8-olato-kappa~2~N,O)boron (PF-06956838) or a pharmaceutically acceptable salt or deuterated analogue thereof.

E301. The method according to any one of embodiments E1 to E99 wherein the boron compound is dimethyl 4-(4,4,5,5-tetramethyl-1,3,2-dioxaborolan-2-yl)pyridine-2,6-dicarboxylate (PHA-00804319) or a pharmaceutically acceptable salt or deuterated analogue thereof.

E302. The method according to any one of embodiments E1 to E99 wherein the boron compound is [(3S)-4-borono-1-hydroxy-1,3-dihydro-2,1-benzoxaborol-3-yl]acetic acid (PF-06961361) or a pharmaceutically acceptable salt or deuterated analogue thereof.

E303. The method according to any one of embodiments E1 to E99 wherein the boron compound is (4S,4aR,5S,5aR,6R,12aS)-4-(dimethylamino)-3,5,10,12,12a-pentahydroxy-9-{[N-(1-hydroxy-3,3-dimethyl-1,3-dihydro-2,1-benzoxaborol-6-yl)glycyl]amino}-6-methyl-1,11-dioxo-1,4,4a,5,5a,6,11,12a-octahydrotet racene-2-carboxamide (PF-06968306) or a pharmaceutically acceptable salt or deuterated analogue thereof.

E304. The method according to any one of embodiments E1 to E99 wherein the boron compound is quinolin-8-yl (3-chloro-4-cyanophenyl){4-[(4-methylpiperazin-1-yl)methyl]phenyl}borinate (PF-06956831) or a pharmaceutically acceptable salt or deuterated analogue thereof.

E305. The method according to any one of embodiments E1 to E99 wherein the boron compound is quinolin-8-yl (4-cyanophenyl){3-[(4-methylpiperazin-1-yl)methyl]phenyl}borinate (PF-06957315) or a pharmaceutically acceptable salt or deuterated analogue thereof.

E306. The method according to any one of embodiments E1 to E99 wherein the boron compound is N-[(6aS,10S,10aR,11S,11aR,12R)-8-carbamoyl-10-(dimethylamino)-4,6,6a,9,11-pentahydroxy-12-methyl-5,7-dioxo-5,6a,7,10,10a,11,11a,12-octahydrotetracen-1-yl]-1-hydroxy-3,3-dimethyl-1,3-dihydro-2,1-benzox aborole-6-carboxamide (PF-06968370) or a pharmaceutically acceptable salt or deuterated analogue thereof.

E307. The method according to any one of embodiments E1 to E99 wherein the boron compound is N-(1-hydroxy-1,3-dihydro-2,1-benzoxaborol-6-yl)benzamide (PF-06957640) or a pharmaceutically acceptable salt or deuterated analogue thereof.

E308. The method according to any one of embodiments E1 to E99 wherein the boron compound is bis(acetato-kappaO)[1-cyclopropyl-6,7-difluoro-8-methyl-4-(oxo-kappaO)-1,4-dihydroquinoline-3-carboxylato-kappaO]boron (PF-04743686) or a pharmaceutically acceptable salt or deuterated analogue thereof.

E309. The method according to any one of embodiments E1 to E99 wherein the boron compound is N-[(6aS,1 0S,1 0aR, 11S, 11aR,12R)-8-carbamoyl-10-(dimethylamino)-4,6,6a,9,11-pentahydroxy-12-methyl-5,7-dioxo-5,6a,7,10,10a,11,11a,12-octahydrotetracen-1-yl]-1-hydroxy-1,3-dihydro-2,1-benzoxaborole-6-car boxamide (PF-06968384) or a pharmaceutically acceptable salt or deuterated analogue thereof.

E310. The method according to any one of embodiments E1 to E99 wherein the boron compound is N-(3-{[(5aR,6aS,7S,10aS)-9-carbamoyl-4,7-bis(dimethylamino)-1,8,10a,11-tetrahydroxy-10,12-dioxo-5,5a,6,6a,7,10,10a,12-octahydrotetracen-2-yl]amino}-3-oxopropyl)-1-hydroxy-1,3-dihydro-2, 1-benzoxaborole -5-carboxamide (PF-06967772) or a pharmaceutically acceptable salt or deuterated analogue thereof.

E311. The method according to any one of embodiments E1 to E99 wherein the boron compound is quinolin-8-yl [3-(4,4-dimethyl-4,5-dihydro-1,3-oxazol-2-yl)phenyl](3-fluorophenyl)borinate (PF-06956596) or a pharmaceutically acceptable salt or deuterated analogue thereof.

E312. The method according to any one of embodiments E1 to E99 wherein the boron compound is {3-[(tert-butoxycarbonyl)amino]propyl}(trifluorido)borate(1-) (PF-06934734) or a pharmaceutically acceptable salt or deuterated analogue thereof.

E313. The method according to any one of embodiments E1 to E99 wherein the boron compound is {3-[benzyl(methyl)amino]prop-1-en-2-yl}(trifluorido)borate(1-) (PF-06840997) or a pharmaceutically acceptable salt or deuterated analogue thereof.

E314. The method according to any one of embodiments E1 to E99 wherein the boron compound is {(2Z)-3-(dimethylamino)-3-hydroxy-1-[4-hydroxy-2-(hydroxy-kappaO)phenyl]prop-2-en-1-onato-kappaO}(difluorido)boron (PF-03806704) or a pharmaceutically acceptable salt or deuterated analogue thereof.

E315. The method according to any one of embodiments E1 to E99 wherein the boron compound is 5-phenoxy-2,1-benzoxaborol-1(3H)-ol (PF-01499456) or a pharmaceutically acceptable salt or deuterated analogue thereof.

E316. The method according to any one of embodiments E1 to E99 wherein the boron compound is bis(4-chlorophenyl)borinic acid (PF-05690484) or a pharmaceutically acceptable salt or deuterated analogue thereof.

E317. The method according to any one of embodiments E1 to E99 wherein the boron compound is (4-{[(3R)-1-(tert-butoxycarbonyl)piperidin-3-yl](3-methylpyridin-2-yl)carbamoyl}phenyl)(trifluorido)borate(1-) (PF-06875313) or a pharmaceutically acceptable salt or deuterated analogue thereof.

E318. The method according to any one of embodiments E1 to E99 wherein the boron compound is N-[(3-methyl-1,2-oxazol-5-yl)methyl]-2-[(3S)-3-(2-methylphenyl)piperidin-1-yl]-7H-pyrrolo[2,3-d]pyrimidin-4-amine (PF-00838447) or a pharmaceutically acceptable salt or deuterated analogue thereof.

E319. The method according to any one of embodiments E1 to E99 wherein the boron compound is N-[(5R,5aR,6S,6aR,7S,10aS)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-1-hydroxy-1,3-dihydro-2, 1-benzoxaborole-6-carboxam ide (PF-06968126) or a pharmaceutically acceptable salt or deuterated analogue thereof.

E320. The method according to any one of embodiments E1 to E99 wherein the boron compound is [(1Z)-1-(dimethylamino)-1-hydroxy-5-(hydroxy-kappaO)-4-phenylpenta-1,4-dien-3-onato-kappaO](difluorido)boron (PF-03696713) or a pharmaceutically acceptable salt or deuterated analogue thereof.

E321. The method according to any one of embodiments E1 to E99 wherein the boron compound is [3-(5-fluoro-2,1-benzoxaborol-1(3H)-yl)phenyl]methyl 8-hydroxyquinoline-2-carboxylate (PF-06957137) or a pharmaceutically acceptable salt or deuterated analogue thereof.

E322. The method according to any one of embodiments E1 to E99 wherein the boron compound is 2-({[bis(3-fluorophenyl)boranyl]oxy}carbonyl)pyridin-3-ol (PF-06956417) or a pharmaceutically acceptable salt or deuterated analogue thereof.

E323. The method according to any one of embodiments E1 to E99 wherein the boron compound is 3-methyl-4-(4,4,5,5-tetramethyl-1,3,2-dioxaborolan-2-yl)benzoic acid (PF-05417781) or a pharmaceutically acceptable salt or deuterated analogue thereof.

E324. The method according to any one of embodiments E1 to E99 wherein the boron compound is 2-(4-bromophenyl)-5,6-dichloro-2H-1,3,2-benzodioxaborole (PF-04009301) or a pharmaceutically acceptable salt or deuterated analogue thereof.

E325. The method according to any one of embodiments E1 to E99 wherein the boron compound is 4,4′-{[1-(nonaboran-1-yl)-1H-borirene-2,3-diyl]bis(methylene)}diphenol (PF-01914444) or a pharmaceutically acceptable salt or deuterated analogue thereof.

E326. The method according to any one of embodiments E288 to E325 wherein the boron compound produces a measurable percent protection in the Droplet Digital Polymerase Chain Reaction (ddPCR) Assay.

E327. The method according to embodiment E326 wherein the boron compound produces a percent protection of at least 10% in the ddPCR assay.

E328. The method according to embodiment E326 wherein the boron compound produces a percent protection of at least 20% in the ddPCR assay.

E329. The method according to embodiment E326 wherein the boron compound produces a percent protection of at least 30% in the ddPCR assay.

E330. The method according to embodiment E326 wherein the boron compound produces a percent protection of at least 40% in the ddPCR assay.

E331. The method according to embodiment E326 wherein the boron compound produces a percent protection of at least 50% in the ddPCR assay.

E332. The method according to embodiment E326 wherein the boron compound produces a percent protection of at least 60% in the ddPCR assay.

E333. The method according to embodiment E326 wherein the boron compound produces a percent protection of at least 70% in the ddPCR assay.

E334. The method according to embodiment E326 wherein the boron compound produces a percent protection of at least 80% in the ddPCR assay.

E335. The method according to embodiment E326 wherein the boron compound produces a percent protection of at least 90% in the ddPCR assay.

E336. The method according to any one of embodiments E288 to E325 wherein the boron compound has an EC₅₀ equal to or less than 700 µM in the ddPCR assay.

E337. The method according to any one of embodiments E288 to E325 wherein the boron compound has an EC₅₀ equal to or less than 600 µM in the ddPCR assay.

E338. The method according to any one of embodiments E288 to E325 wherein the boron compound has an EC₅₀ equal to or less than 500 µM in the ddPCR assay.

E339. The method according to any one of embodiments E288 to E325 wherein the boron compound has an EC₅₀ equal to or less than 400 µM in the ddPCR assay.

E340. The method according to any one of embodiments E288 to E325 wherein the boron compound has an EC₅₀ equal to or less than 300 µM in the ddPCR assay.

E341. The method according to any one of embodiments E288 to E325 wherein the boron compound has an EC₅₀ equal to or less than 200 µM in the ddPCR assay.

E342. The method according to any one of embodiments E288 to E325 wherein the boron compound has an EC₅₀ equal to or less than 100 µM in the ddPCR assay.

E343. The method according to any one of embodiments E288 to E325 wherein the boron compound has an EC₅₀ equal to or less than 50 µM in the ddPCR assay.

E344. The composition according to any one of embodiments E225 to E275 wherein the boron compound is any of the compounds shown in Table 1, or a pharmaceutically acceptable salt or deuterated analogue thereof.

E345. The composition according to any one of embodiments E225 to E278, or embodiment E344 wherein the RNA and boron compound are contained within a non-viral vector.

E346. The composition according to any one of embodiments E225 to E278, or embodiment E344 wherein the RNA and boron compound are contained within a nanoparticle.

E347. The composition according to any one of embodiments E225 to E278, or embodiment E344 wherein the RNA and boron compound are contained within a liposome.

E348. The composition according to any one of embodiments E225 to E278, or embodiment E344 wherein the RNA and boron compound are contained within a micelle.

E349. The composition according to any one of embodiments E225 to E278, or embodiment E344 wherein the RNA and boron compound are contained within a viral vector.

E350. The composition according to any one of embodiments E225 to E278, or embodiment E344 wherein the RNA and boron compound are contained within an Adenovirus.

E351. The composition according to any one of embodiments E225 to E278, or embodiment E344 wherein the RNA and boron compound are contained within an adeno-associated virus.

E352. The composition according to any one of embodiments E225 to E278, or embodiment E344 wherein the RNA and boron compound are contained within a retrovirus.

E353. The composition according to any one of embodiments E225 to E278, or embodiment E344 wherein the RNA and boron compound are contained within a lentivirus.

### Definitions

The term “boron compound,” as used herein, means a compound with a chemical structure that contains at least one boron atom. The boron compounds may be a free base or a free acid or a pharmaceutically acceptable salt thereof. Examples of boron compounds that are useful for protecting RNA from degrading include, but are not limited to, the boron compounds disclosed in WO2004/056322, WO2005/013892, WO2005/123094, WO2006/007384, WO2006/007384, WO2006/085932, WO2006/089067, WO2007/078340, WO2007/095638, WO2007/131072, WO2007/146965, WO2007/079119, WO2008/157726, WO2009/111676, WO2009/140309, WO2010/028005, WO2010/027975, WO2010/045503, WO2010/045505, WO2010/080558, WO2011/049971, WO2011/017125, WO2011/019618, WO2011/022337, WO2011/037731, WO2011/060196, WO2011/060199, WO2011/094450, WO2011/116348, WO2012/033858, WO2013/078070, WO2013/078071, WO2014/121124, WO2014/149793, WO2015/013318, WO2015/042532, WO2018/160845, WO2020/070651, US7465836, US8106031, US8623911, and US9346834, all of which are hereby incorporated by reference in their entirety.

The terms “protect” or “protection” or “protecting” or “protective effect” as used herein, means slowing, reducing, or eliminating RNA degradation after contacting the RNA with a quantity of one or more boron compounds. The protective effect may be measured by assays including, but not limited to, RNA Fluorescence Polarization Protection Assay, Syto-9 Dye Intercalation Assay, Droplet Digital Polymerase Chain Reaction Assay, and TapeStation Assay.

The term “RNA,” as used herein, means a sequence of nucleotides comprising a nucleotide base adenine (A), cytosine (C), guanine (G), and uracil (U) attached to a sugar such as ribose attached to a phosphate group wherein the nucleotide base, sugar, or phosphate may be modified wherein such modifications are known to those skilled in the art. It is to be understood that this definition includes single strand RNAs and double stranded RNAs.

The term “RNA degradation,” as used herein, means the original RNA nucleotide sequence is no longer intact. RNA may be degraded under certain conditions including, but not limited to, pH or temperature, or both, over time. In particular, acidic pH conditions, basic pH conditions and/or increased temperatures may accelerate RNA degradation over time.

The term “acidic conditions” or “acidic pH conditions,” as used herein, means a pH less than 7.0.

The term “basic conditions” or “basic pH conditions,” as used herein, means a pH greater than 7.0, preferably a pH greater than 10.0, most preferred a pH greater than 11.0.

The term “pharmaceutically acceptable salt,” as used herein, means those salts within the scope of sound medical judgement that are suitable for use in contact with the tissues of humans and animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66:1-19, herein incorporated by reference. The salts can be prepared in situ during the final isolation and purification of the compounds of the present invention or separately by reacting a free base (basic nitrogen) with a suitable organic or inorganic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. The term “pharmaceutically acceptable salt,” as used herein, also means salts of carboxylic acid and sulfonic acid groups prepared by reacting the free acid with a positively charged inorganic or organic ion (cation) that is generally considered suitable for human consumption. Examples of pharmaceutically acceptable cations are lithium, sodium, potassium, magnesium, calcium, ferrous, ferric, ammonium, alkylammonium, dialkylammonium, trialkylammonium, tetraalkylammonium, diethanolammmonium, and choline. Cations may be interchanged by methods known in the art, such as ion exchange. Where compounds of the present invention contain one or more carboxylic acid groups or sulfonic acid groups, addition of a base (such as a hydroxide or a free amine) will yield the appropriate cationic form.

### Synthesis of Boron Compounds

The boron compounds useful for the methods and compositions of the present invention were prepared using synthetic strategies known to those skilled in the art and by the synthetic strategies disclosed in WO2004/056322, WO2005/013892, WO2005/123094, WO2006/007384, WO2006/007384, WO2006/085932, WO2006/089067, WO2007/078340, WO2007/095638, WO2007/131072, WO2007/146965, WO2007/079119, WO2008/157726, WO2009/111676, WO2009/140309, WO2010/028005, WO2010/027975, WO2010/045503, WO2010/045505, WO2010/080558, WO2011/049971, WO2011/017125, WO2011/019618, WO2011/022337, WO2011/037731, WO2011/060196, WO2011/060199, WO2011/094450, WO2011/116348, WO2012/033858, WO2013/078070, WO2013/078071, WO2014/121124, WO2014/149793, WO2015/013318, WO2015/042532, WO2018/160845, WO2020/070651, US7465836, US8106031, US8623911, and US9346834, all of which are hereby incorporated by reference in their entirety.

Boron compounds useful in the methods and compositions of the present invention can exist as stereoisomers, wherein asymmetric or chiral centers are present. Stereoisomers are designated (R) or (S), depending on the configuration of substituents around the chiral carbon atom. The terms (R) and (S) used herein are configurations as defined in IUPAC 1974 Recommendations for Section E, Fundamental Stereochemistry, Pure Appl. Chem., (1976), 45: 13-30. The present invention contemplates various stereoisomers and mixtures thereof and are specifically included within the scope of this invention. Stereoisomers include enantiomers, diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution, a technique well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) direct separation of the mixture of optical enantiomers on chiral chromatographic columns, or (3) formation of a diastereomeric salt followed by selective recrystallization of one of the diastereomeric salts.

Boron compounds useful in the methods and compositions of the present invention that contain at least one double bond (olefins) may be designated as trans (E) or cis (Z). For example, 3-(4-fluorobenzyl)-5-(4-(((1-hydroxy-1,3-dihydrobenzo[c][1,2]oxaborol-6-yl)oxy)methyl)benzylidene)thiazolidine-2,4-dione (PF-06963029) may exist as the (E) or (Z) olefin or as a combination of both (E) and (Z) olefins.

Boron compounds useful in the methods and compositions of the present invention includes “deuterated analogues” of the boron compounds wherein from 1 to n hydrogens attached to a carbon atom is/are replaced by deuterium, wherein n is the number of hydrogens in the molecule. The deuterated boron compounds of the present invention are synthesized by means well known in the art, for example by employing starting materials in which one or more hydrogens have been replaced by deuterium.

Boron compounds useful in the methods and compositions of the present invention and disclosed in Table 1 were named by Chemdraw® Professional version 18.0.0.231 (4029) or were given names which appeared to be consistent with Chemdraw® nomenclature.

TABLE 1

| | | |
|---|---|---|
| PF-Number | Structure | Nomenclature |
| PF-06957640 | 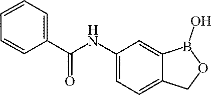 [SMILES: O=C(Nc1ccc2c(c1)B(O)OC2)c1ccccc1] | N-(1-hydroxy-1,3-dihydrobenzo[c][1,2]oxabor ol-6-yl)benzamide |
| PF-07230998 | 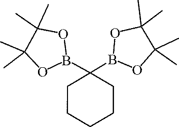 | 2,2′-(cyclohexane-1,1-diyl)bis(4,4,5,5-tetramethyl-1,3,2-dioxaborolane) |
| PF-06967658 | 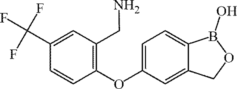 [SMILES: NCc1cc(C(F)(F)F)ccc1Oc1ccc2c(c1)COB2O] | 5-(2-(aminomethyl)-4-(trifluoromethyl)phenoxy)be nzo[c][1,2]oxaborol-1 (3H)-ol |
| PF-06966741 | 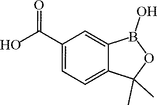 | 1-hydroxy-3,3-dimethyl-1,3-dihydrobenzo[c][1,2]oxabor ole-6-carboxylic acid |
| PF-06963078 | | N-(3-(1-hydroxy-1,3-dihydrobenzo[c][1,2]oxabor ol-4-yl)phenyl)-5-(1-methyl- |
| | 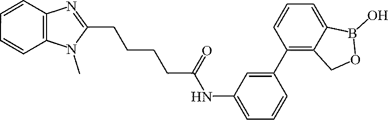 [SMILES: Cn1c(CCCCC(=O)Nc2cccc(-c3cccc4c3COB4O)c2)nc2ccccc21] | 1H-benzo[d]imidazol-2-yl)pentanamide |
| PF-06962739 | 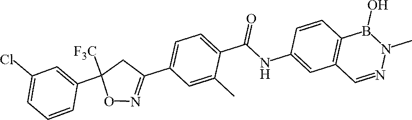 [SMILES: Cc1cc(C2=NOC(c3cccc(Cl)c3)(C(F)(F)F)C2)ccc1C(=O)Nc1ccc2c(c1)C=NN(C)B2O] | 4-(5-(3-chlorophenyl)-5-(trifluoromethyl)-4,5-dihydroisoxazol-3-yl)-N-(1-hydroxy-2-methyl-1,2-dihydrobenzo[d][1,2,3]diaza borinin-6-yD-2-methylbenzamide |
| PF-06963029 | 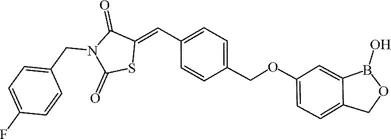 [SMILES: O=C1S/C(=C\c2ccc(COc3ccc4c(c3)B(O)OC4)cc2)C(=O)N1Cc1ccc(F)cc1] | 3-(4-fluorobenzyl)-5-(4-(((1-hydroxy-1,3-dihydrobenzo[c][1,2]oxabor ol-6-yl)oxy)methyl)benzylidene)t hiazolidine-2,4-dione |
| PF-06962691 | 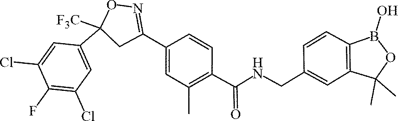 [SMILES: Cc1cc(C2=NOC(c3cc(Cl)c(F)c(Cl)c3)(C(F)(F)F)C2)ccc1C(=O)NCc1ccc2c(c1)C(C)(C)OB2O] | 4-(5-(3,5-dichloro-4-fluorophenyl)-5-(trifluoromethyl)-4,5-dihydroisoxazol-3-yl)-N-((1-hydroxy-3,3-dimethyl-1,3-dihydrobenzo[c][1,2]oxabor ol-5-yl)methyl)-2-methylbenzamide |
| PF-06959336 | 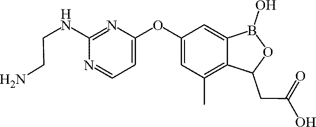 [SMILES: Cc1cc(Oc2ccnc(NCCN)n2)cc2c1C(CC(=O)O)OB2O] | 2-(6-((2-((2-aminoethyl)amino)pyrimidin -4-yl)oxy)-1-hydroxy-4-methyl-1,3-dihydrobenzo[c][1,2]oxabor ol-3-yl)acetic acid |
| PF-01499456 | 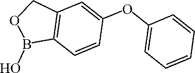 [SMILES: OB1OCc2cc(Oc3ccccc3)ccc21] | 5-phenoxybenzo[c][1,2]oxabo rol-1 (3H)-ol |
| PF-06957808 | 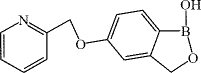 [SMILES: OB1OCc2cc(OCc3ccccn3)ccc21] | 5-(pyridin-2-ylmethoxy)benzo[c][1,2]oxa borol-1 (3H)-ol |
| PF-06957880 | 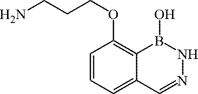 [SMILES: NCCCOc1cccc2c1B(O)NN=C2] | 8-(3-aminopropoxy)benzo[d][1,2, 3]diazaborinin-1 (2H)-ol |
| PF-06957807 | | 4-(aminomethyl)-N-(1-hydroxy-1,3- |
| | 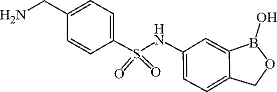 [SMILES: NCc1ccc(S(=O)(=O)Nc2ccc3c(c2)B(O)OC3)cc1] | dihydrobenzo[c][1,2]oxabor ol-6-yl)benzenesulfonamide |
| PF-06969336 | 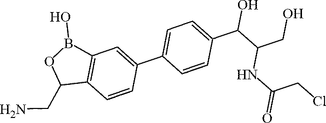 [SMILES: NCC1OB(O)c2cc(-c3ccc(C(O)C(CO)NC(=O)CCl)cc3)ccc21] | N-((1R,2R)-1-(4-((S)-3-(aminomethyl)-1-hydroxy-1,3-dihydrobenzo[c][1,2]oxabor ol-6-yQphenyQ-1,3-dihydroxypropan-2-yl)-2-chloroacetamide |
| PF-06967791 | 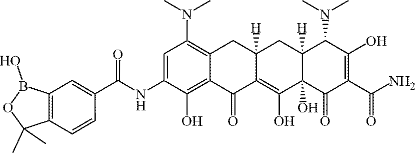 [SMILES: CN(C)c1cc(NC(=O)c2ccc3c(c2)B(O)OC3(C)C)c(O)c2c1C[C@H]1C[C@H]3[C@H](N(C)C)C(O)=C(C(N)=O)C(=O)[C@@]3(O)C(O)=C1C2=O] | N-[(5aR,6aS,7S,1 OaS)-9-carbamoyl-4,7-bis(dimethylamino)-1,8,10a,11-tetrahydroxy-10,12-dioxo-5,5a,6,6a,7,10,10a,12-octahydrotetracen-2-yl]-1-hydroxy-3,3-dimethyl-1,3-dihydro-2,1-benzoxaborole-6-carboxamide |
| PF-06968419 | 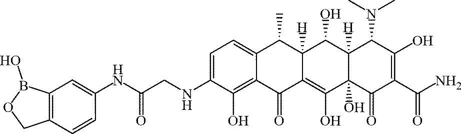 [SMILES: C[C@H]1c2ccc(NCC(=O)Nc3ccc4c(c3)B(O)OC4)c(O)c2C(=O)C2=C(O)[C@]3(O)C(=O)C(C(N)=O)=C(O)[C@@H](N(C)C)[C@@H]3[C@@H](O)[C@@H]21] | (4S,4aR,5S,5aR,6R,12aS)-4-(dimethylamino)-3,5,10,12,12a-pentahydroxy-9-({2-[(1-hydroxy-1,3-dihydro-2,1-benzoxaborol-6-yl)amino]-2-oxoethyl}amino)-6-methyl-1,11-dioxo-1,4,4a,5,5a,6,11,12a-octahydrotetr acene-2-carboxamide |
| PF-06968256 | 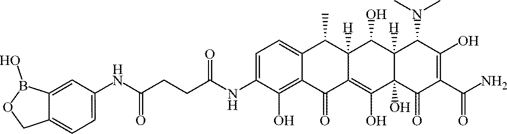 [SMILES: C[C@H]1c2ccc(NC(=O)CCC(=O)Nc3ccc4c(c3)B(O)OC4)c(O)c2C(=O)C2=C(O)[C@]3(O)C(=O)C(C(N)=O)=C(O)[C@@H](N(C)C)[C@@H]3[C@@H](O)[C@@H]21] | N-1-[(5R,5aR,6S,6aR,7S,10aS)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-N~4~-(1-hydroxy-1,3-dihydro-2,1-benzoxaborol-6-yl)butanediamide |
| PF-06968559 | 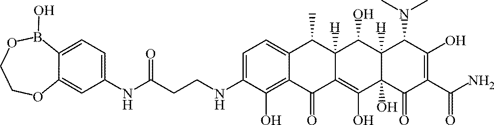 [SMILES: C[C@H]1c2ccc(NCCC(=O)Nc3ccc4c(c3)OCCOB4O)c(O)c2C(=O)C2=C(O)[C@]3(O)C(=O)C(C(N)=O)=C(O)[C@@H](N(C)C)[C@@H]3[C@@H](O)[C@@H]21] | (4S,4aR,5S,5aR,6R,12aS)-4-(dimethylamino)-3,5,10,12,12a-pentahydroxy-9-({3-[(1-hydroxy-3,4-dihydro-1H-2,5,1-benzodioxaborepin-7- |
| | | yl)amino]-3-oxopropyl}amino)-8-methyl-1,11-dioxo-1,4,4a,5,5a,6,11,12a-oc tahydrotetracene-2-carboxamide |
| PF-06968542 | 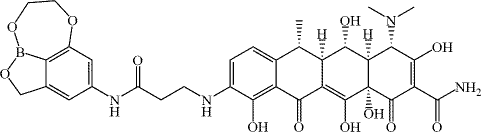 [SMILES: C[C@H]1c2ccc(NCCC(=O)Nc3cc4c5c(c3)OCCOB5OC4)c(O)c2C(=O)C2=C(O)[C@]3(O)C(=O)C(C(N)=O)=C(O)[C@@H](N(C)C)[C@@H]3[C@@H](O)[C@@H]21] | (4S,4aR,5S,5aR,6R,12aS)-9-({3-[(2,3-dihydro-7H-[1,2]oxaborolo[4,3,2-jk][2,5,1]benzodioxaborepin -9-yl)amino]-3-oxopropyl}amino)-4-(dimethylamino)-3,5,10,12,12a-pentahydroxy-6-methyl-1,11-dioxo-1,4,4a,5,5a,6,11,12a-octahydrotetracene-2-carboxamide |
| PF-06956816 | 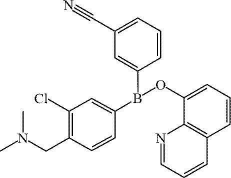 [SMILES: CN(C)Cc1ccc(B(Oc2cccc3cccnc23)c2cccc(C#N)c2)cc1Cl] | quinolin-8-yl {3-chloro-4-[(dimethylamino)methyl]phe nyl}(3-cyanophenyl)borinate |
| PF-06956810 | 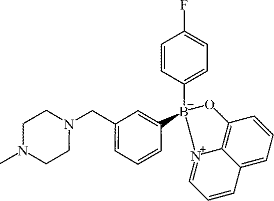 [SMILES: CN1CCN(Cc2cccc([B@@-]3(c4ccc(F)cc4)Oc4cccc5ccc[n+]3c45)c2)CC1] | (4-fluorophenyl){3-[(4-methylpiperazin-1-yl)methyl]phenyl}(quinolin-8-olato-kappa-2-N,O)boron |
| PF-06968101 | 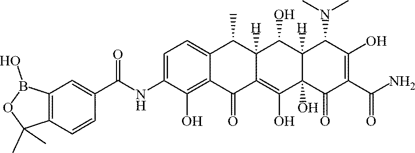 [SMILES: C[C@H]1c2ccc(NC(=O)c3ccc4c(c3)B(O)OC4(C)C)c(O)c2C(=O)C2=C(O)[C@]3(O)C(=O)C(C(N)=O)=C(O)[C@@H](N(C)C)[C@@H]3[C@@H](O)[C@@H]21] | N-[(5R,5aR,6S,6aR,7S,10aS)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-1-hydroxy-3,3-dimethyl-1,3-dihydro-2,1-benzoxaboro le-6-carboxamide |
| PF-06968237 | 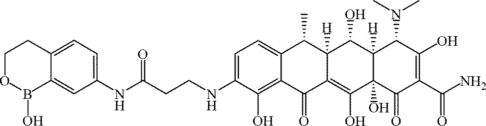 [SMILES: C[C@H]1c2ccc(NCCC(=O)Nc3ccc4c(c3)B(O)OCC4)c(O)c2C(=O)C2=C(O)[C@]3(O)C(=O)C(C(N)=O)=C(O)[C@@H](N(C)C)[C@@H]3[C@@H](O)[C@@H]21] | (4S,4aR,5S,5aR,6R,12aS)-4-(dimethylamino)-3,5,10,12,12a-pentahydroxy-9-({3-[(1-hydroxy-3,4-dihydro-1H-2,1-benzoxaborinin-7-yl)amino]-3-oxopropyl}amino)-6- |
| | | methyl-1,11-dioxo-1,4,4a,5,5a,6,11,12a-octahyd rotetracene-2-carboxamide |
| PF-06956823 | 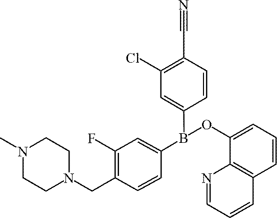 [SMILES: CN1CCN(Cc2ccc(B(Oc3cccc4cccnc34)c3ccc(C#N)c(Cl)c3)cc2F)CC1] | quinolin-8-yl (3-chloro-4-cyanophenyl){3-fluoro-4-[(4-methylpiperazin-1-yl)methyl]phenyl}borinate |
| PF-06968488 | 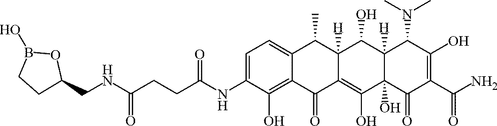 [SMILES: C[C@H]1c2ccc(NC(=O)CCC(=O)NC[C@H]3CCB(O)O3)c(O)c2C(=O)C2=C(O)[C@]3(O)C(=O)C(C(N)=O)=C(O)[C@@H](N(C)C)[C@@H]3[C@@H](O)[C@@H]21] | N~1~-[(5RS,5aRS,6SR,6aRS,7S R,10aSR)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-N~4~-{[(5RS)-2-hydroxy-1,2-oxaborolan-5-y I]methyl}butanediamide |
| PF-06956838 | 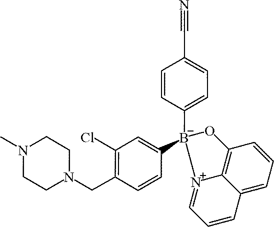 [SMILES: CN1CCN(Cc2ccc([B@@-]3(c4ccc(C#N)cc4)Oc4cccc5ccc[n+]3c45)cc2Cl)CC1] | {3-chloro-4-[(4-methylpiperazin-1-yl)methyl]phenyl}(4-cyanophenyl)(quinolin-8-olato-kappa-2-N,O)boron |
| PHA-00804319 | 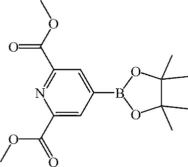 | dimethyl 4-(4,4,5,5-tetramethyl-1,3,2-dioxaborolan-2-yl)pyridine-2,6-dicarboxylate |
| PF-06961361 | 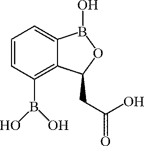 [SMILES: O=C(O)C[C@@H]1OB(O)c2cccc(B(O)O)c21] | [(3S)-4-borono-1-hydroxy-1,3-dihydro-2,1-benzoxaborol-3-yl]acetic acid |
| PF-06968306 | 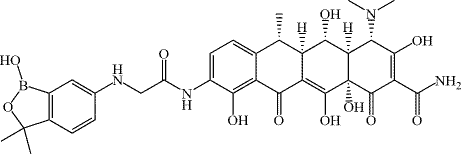 [SMILES: C[C@H]1c2ccc(NC(=O)CNc3ccc4c(c3)B(O)OC4(C)C)c(O)c2C(=O)C2=C(O)[C@]3(O)C(=O)C(C(N)=O)=C(O)[C@@H](N(C)C)[C@@H]3[C@@H](O)[C@@H]21] | (4S,4aR,5S,5aR,6R,12aS)-4-(dimethylamino)-3,5,10,12,12a-pentahydroxy-9-{[N-(1-hydroxy-3,3-dimethyl-1,3-dihydro-2,1-benzoxaborol-6-yl)glycyl]amino}-6-methyl-1,11-dioxo-1,4,4a,5,5a,6,11,12a-octahydrotet racene-2-carboxamide |
| PF-06956831 | 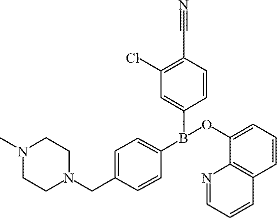 [SMILES: CN1CCN(Cc2ccc(B(Oc3cccc4cccnc34)c3ccc(C#N)c(Cl)c3)cc2)CC1] | quinolin-8-yl (3-chloro-4-cyanophenyl){4-[(4-methylpiperazin-1-yl)methyl]phenyl}borinate |
| PF-06957315 | 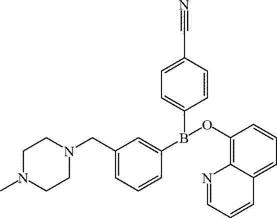 [SMILES: CN1CCN(Cc2cccc(B(Oc3cccc4cccnc34)c3ccc(C#N)cc3)c2)CC1] | quinolin-8-yl (4-cyanophenyl){3-[(4-methylpiperazin-1-yl)methyl]phenyl}borinate |
| PF-06968370 | 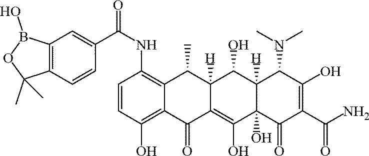 [SMILES: C[C@H]1c2c(NC(=O)c3ccc4c(c3)B(O)OC4(C)C)ccc(O)c2C(=O)C2=C(O)[C@]3(O)C(=O)C(C(N)=O)=C(O)[C@@H](N(C)C)[C@@H]3[C@@H](O)[C@@H]21] | N-[(6aS,10S,10aR,11S,11aR, 12R)-8-carbamoyl-10-(dimethylamino)-4,6,6a,9,11-pentahydroxy-12-methyl-5,7-dioxo-5,6a,7,10,10a,11,11a,12-octahydrotetracen-1-yl]-1-hydroxy-3,3-dimethyl-1,3-dihydro-2,1-benzox aborole-6-carboxamide |
| PF-06957640 | 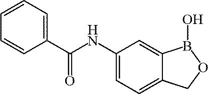 [SMILES: O=C(Nc1ccc2c(c1)B(O)OC2)c1ccccc1] | N-(1-hydroxy-1,3-dihydro-2,1-benzoxaborol-6-yl)benzamide |
| PF-04743686 | 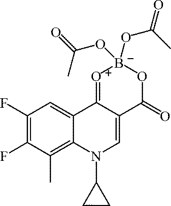 | bis(acetato-kappaO)[1-cyclopropyl-6,7-difluoro-8-methyl-4-(oxo-kappaO)-1,4-dihydroquinoline-3-carboxylato-kappaO]boron |
| PF-06968384 | 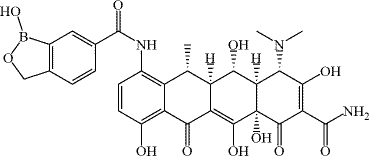 [SMILES: C[C@H]1c2c(NC(=O)c3ccc4c(c3)B(O)OC4)ccc(O)c2C(=O)C2=C(O)[C@]3(O)C(=O)C(C(N)=O)=C(O)[C@@H](N(C)C)[C@@H]3[C@@H](O)[C@@H]21] | N-[(6aS,10S,10aR,11S,11aR, 12R)-8-carbamoyl-10-(dimethylamino)-4,6,6a,9,11-pentahydroxy-12-methyl-5,7-dioxo-5,6a,7,10,10a,11,11a,12-octahydrotetracen-1-yl]-1-hydroxy-1,3-dihydro-2,1-benzoxaborole-6-car boxamide |
| PF-06967772 | 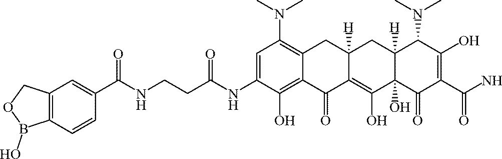 [SMILES: CN(C)c1cc(NC(=O)CCNC(=O)c2ccc3c(c2)COB3O)c(O)c2c1C[C@H]1C[C@H]3[C@H](N(C)C)C(O)=C(C(N)=O)C(=O)[C@@]3(O)C(O)=C1C2=O] | N-(3-{[(5aR,6aS,7S,10aS)-9-carbamoyl-4,7-bis(dimethylamino)-1,8,10a,11-tetrahydroxy-10,12-dioxo-5,5a,6,6a,7,10,10a,12-octahydrotetracen-2-yl]amino}-3-oxopropyl)-1-hydroxy-1,3-dihydro-2,1-benzoxaborole -5-carboxamide |
| PF-06956596 | 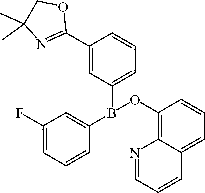 [SMILES: CC1(C)COC(c2cccc(B(Oc3cccc4cccnc34)c3cccc(F)c3)c2)=N1] | quinolin-8-yl [3-(4,4-dimethyl-4,5-dihydro-1,3-oxazol-2-yl)phenyl](3-fluorophenyl)borinate |
| PF-06934734 | 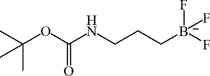 | {3-[(tert-butoxycarbonyl)amino]prop yl}(trifluorido)borate(1-) |
| PF-06840997 | 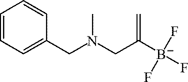 [SMILES: C=C(CN(C)Cc1ccccc1)[B-](F)(F)F] | {3-[benzyl(methyl)amino]prop-1-en-2-yl}(trifluorido)borate(1-) |
| PF-03806704 | 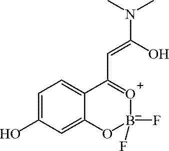 [SMILES: CN(C)/C(O)=C/C1=[O+][B-](F)(F)Oc2cc(O)ccc21] | {(2Z)-3-(dimethylamino)-3-hydroxy-1-[4-hydroxy-2-(hydroxy-kappaO)phenyl]prop-2-en-1-onato-kappaO}(difluorido)boron |
| PF-01499456 | 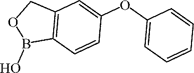 [SMILES: OB1OCc2cc(Oc3ccccc3)ccc21] | 5-phenoxy-2,1-benzoxaborol-1 (3H)-ol |
| PF-05690484 | 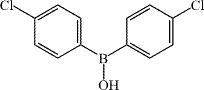 [SMILES: OB(c1ccc(Cl)cc1)c1ccc(Cl)cc1] | bis(4-chlorophenyl)borinic acid |
| PF-06875313 | 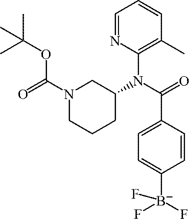 [SMILES: Cc1cccnc1N(C(=O)c1ccc([B-](F)(F)F)cc1)[C@@H]1CCCN(C(=O)OC(C)(C)C)C1] | (4-{[(3R)-1-(tert-butoxycarbonyl)piperidin-3-yl](3-methylpyridin-2-yl)carbamoyl}phenyl)(trifluor ido)borate(1-) |
| PF-00838447 | 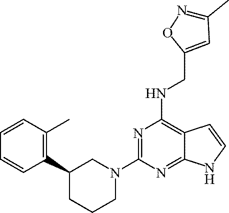 [SMILES: Cc1cc(CNc2nc(N3CCC[C@@H](c4ccccc4C)C3)nc3[nH]ccc23)on1] | N-[(3-methyl-1,2-oxazol-5-yl)methyl]-2-[(3S)-3-(2-methylphenyl)piperidin-1-yl]-7H-pyrrolo[2,3-d]pyrimidin-4-amine |
| PF-06968126 | 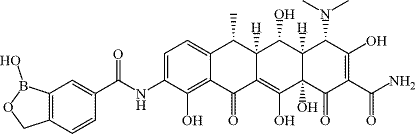 [SMILES: C[C@H]1c2ccc(NC(=O)c3ccc4c(c3)B(O)OC4)c(O)c2C(=O)C2=C(O)[C@]3(O)C(=O)C(C(N)=O)=C(O)[C@@H](N(C)C)[C@@H]3[C@@H](O)[C@@H]21] | N-[(5R,5aR6S,6aR,7S,10aS)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-1-hydroxy-1,3-dihydro-2,1-benzoxaborole-6-carboxam ide |
| PF-03696713 | 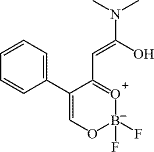 [SMILES: CN(C)/C(O)=C/C1=[O+][B-](F)(F)OC=C1c1ccccc1] | [(1Z)-1-(dimethylamino)-1-hydroxy-5-(hydroxy-kappaO)-4-phenylpenta-1,4-dien-3-onato-kappaO](difluorido)boron |
| PF-06957137 | 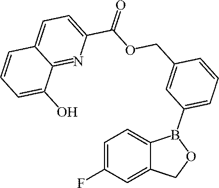 [SMILES: O=C(OCc1cccc(B2OCc3cc(F)ccc32)c1)c1ccc2cccc(O)c2n1] | [3-(5-fluoro-2,1-benzoxaborol-1 (3H)-yl)phenyl]methyl 8-hydroxyquinoline-2-carboxylate |
| PF-06956417 | 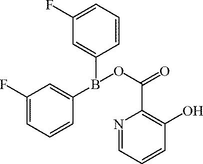 [SMILES: O=C(OB(c1cccc(F)c1)c1cccc(F)c1)c1ncccc1O] | 2-({[bis(3-fluorophenyl)boranyljoxy}ca rbonyl)pyridin-3-ol |
| PF-05417781 | 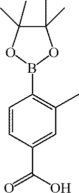 | 3-methyl-4-(4,4,5,5-tetramethyl-1,3,2-dioxaborolan-2-yl)benzoic acid |
| PF-04009301 | 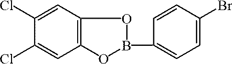 [SMILES: Clc1cc2c(cc1Cl)OB(c1ccc(Br)cc1)O2] | 2-(4-bromophenyl)-5,6-dichloro-2H-1,3,2-benzodioxaborole |
| PF-01914444 | 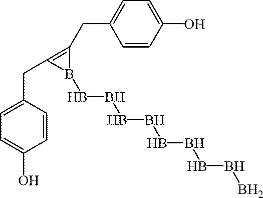 | 4,4′-{[1-(nonaboran-1-yl)-1H-borirene-2,3-diyl]bis(methylene)}diphenol |

### Biological Experiments Performed to Identify and Characterize Boron Compounds that Protect RNA

To identify boron compounds that showed utility in protecting RNA from degradation, several functional assays were used with multiple RNA constructs. All functional assays were performed under conditions that accelerate RNA degradation; notably incubation of RNA in basic pH buffer at high temperature over time. These assays were performed with boron compounds and compared to a) no-degradation baseline control and b) degradation in presence of compound solvating agent, dimethyl sulfoxide (DMSO) alone (e.g. no compound) controls, to gauge degree of protection imparted by the boron compound. Multiple functional assay approaches were used where differing endpoints were measured including a ddPCR assay where RNA sequence was determined, an RNA fluorescence polarization (FP) assay that measures fluorescent probe activity to gauge intact (high millipolarization signal) and degraded (low millipolarization signal), a Syto-9 dye intercalation assay where disruption in amount of dye available to bind RNA backbone infers instability, and finally, a TapeStation assay that utilizes capillary electrophoresis and optical measurement to measure RNA. Prototypical boron compounds that showed protection may be further characterized using assays that assess binding to demonstrate direct interaction of the boron compound binding to RNA. Affinity selection by mass spectrometry (ASMS), native electrospray ionization mass spectrometry (nESI-MS) and nuclear magnetic resonance (NMR) may be used as several complementary approaches to ascertain compound binding to RNA. The latter techniques utilize MS to measure mass of bound compound alone (ASMS), or mass of RNA and bound compound (nESI-MS). The nESI-MS and NMR studies may be conducted to provide direct analytical quantification of resulting complex via measurement of mass (nESI-MS) or chemical shifts (NMR). The ASMS experiment is conducted to determine saturation binding of boron to RNA where resulting apparent binding constants (K_{D,App}) may be generated. Further descriptions of the functional and binding assays are provided in the section below.

### Droplet Digital Polymerase Chain Reaction (ddPCR) Assay

The ddPCR assays measured RNA integrity by converting intact RNA to cDNA by reverse transcription, then performing digital PCR in thousands of nanoliter-sized droplets for each sample. PCR was performed within each droplet, which allowed for more sensitive detection of subtle changes in RNA concentration compared to traditional PCR or qPCR. Additionally, primers/probes specific to the 5′ end of the RNA/cDNA transcript(s) were used to assess fragment integrity, i.e. degraded RNA had fewer 5′ positive droplets. The BioRad QX200 Droplet Digital PCR System was used for the ddPCR workflow.

RNA samples (Chi18 or HCV IRES) were diluted to 100 fg/µL in 50 mM Tris buffer pH 11.4. Boron compounds were tested in a dose-response format to determine EC₅₀. A compound dilution plate that contained a 30 mM top concentration of compound in DMSO was serially (half-log) diluted 10 times. A quantity of 0.12 µL of this compound dilution plate was spotted into a 96-well PCR plate (Thermo). Sample RNAs were added (6 µL) and incubated with test compounds as described below. Final compound concentrations ranged from 600 µM to 0.018 µM in 2% DMSO.

In addition, each PCR plate also contained positive (non-degraded) and negative (degraded) control wells to define the upper and lower limits for the assay signal. These control wells did not contain test compound but instead received 6 µL of RNA samples treated with DMSO (2% final) on the same day as the assay (100% protection, non-degraded control) or 6 µL of RNA samples treated with DMSO (2% final) on the same day as compound treatment to define 0% protection, degraded control.

For the degradation reaction, treated RNAs were incubated for 16 hrs at 30° C. for Chi18 samples or 42° C. for HCV samples. Upon completion of the degradation reaction, cDNA was made and ddPCR run for each sample (96 well plates). Positive 5′ droplet values were divided by the total accepted droplet values for each sample and a ratio value generated for each sample.

Data was imported into a database where percent (%) protection and compound EC₅₀ were calculated and curated. Percent protection was calculated by using the following formula: % protection = 100 × (ZPE-sample)/(ZPE-HPE) where ZPE (zero % protection) was the signal observed in wells that contained RNA with 2% DMSO (no compound) degraded over night for 16 hrs under conditions described previously, HPE (high percent effect) was the signal observed in wells that contained RNA with 2% DMSO that was not subjected to degradation conditions overnight, and ‘sample’ was the signal observed in wells that contained the indicated concentrations of compound. EC₅₀ was determined by using a four parameter logistic equation: Y=A+((B-A)/(1+(C/X)D)); where Y is percent protection, X is compound concentration, A and B are minimum and maximum asymptotes, respectively, C is EC₅₀, and D is Hill slope.

EC50 values (µM) were determined using the ddPCR assay for the compounds shown in Table 2.

TABLE 2

EC50 Values Obtained from the ddPCR Assay

| Compound | EC50 (µM) |
|---|---|
| PF-06967791 | 55.640 |
| PF-06968419 | 75.125 |
| PF-06968256 | 75.661 |
| PF-06968559 | 99.066 |
| PF-06959336 | 113.962 |
| PF-06968542 | 125.725 |
| PF-06956816 | 136.675 |
| PF-06956810 | 140.243 |
| PF-06968101 | 161.767 |
| PF-06968237 | 165.738 |
| PF-06956823 | 174.586 |
| PF-06968488 | 188.930 |
| PF-06956838 | 207.010 |
| PHA-00804319 | 214.196 |
| PF-06961361 | 225.638 |
| PF-06968306 | 234.037 |
| PF-06956831 | 242.095 |
| PF-06957315 | 252.545 |
| PF-06968370 | 258.082 |
| PF-06957640 | 266.385 |
| PF-04743686 | 271.146 |
| PF-06968384 | 280.988 |
| PF-06967772 | 286.082 |
| PF-06956596 | 300.446 |
| PF-06934734 | 301.763 |
| PF-06840997 | 305.744 |
| PF-03806704 | 322.882 |
| PF-01499456 | 330.247 |
| PF-05690484 | 354.799 |
| PF-06875313 | 359.502 |
| PF-00838447 | 388.373 |
| PF-06968126 | 392.858 |
| PF-03696713 | 399.575 |
| PF-06957137 | 429.310 |
| PF-06956417 | 430.177 |
| PF-05417781 | 462.819 |
| PF-04009301 | 474.164 |
| PF-01914444 | 488.021 |

### Affinity Selection by Mass Spectrometry (ASMS) Binding Protocol

In the ASMS assay, compounds are incubated with target of interest (protein, RNA, etc.) over a timescale that allowed for equilibrium binding to occur. The resulting samples are injected into a 2-dimensional liquid chromatographer, where dimension 1 is size-exclusion and the binder + target is selected. From there, samples flow through a column heater to promote release of the binder from the target. These binders pass through the second reverse phase chromatography dimension. Finally, these samples are injected into a high-resolution time of flight mass spectrometer following electrospray ionization where binders in the samples are analyzed and quantified.

Boron compounds are pooled into plate wells in DMSO to yield final compound concentration of 0.5 mM, with 200 nL total DMSO per incubation (“well”). Five mL of RNA solution of interest is added to each well of plate well with a Formulatrix Mantis plate dispensing robot and incubate for 2 hours at ambient temperature. Plates are then centrifuged at 2000 g for 10 minutes (min) at 4° C. before loading into HPLC auto sampler at 4° C. Final assay concentrations are as follows: 20 µM each compound and 0.5-10 µM of RNA. Two microliters of each sample are injected into a 2-D chromatography system consisting of two Agilent HPLCs and one Agilent G6549A QTOF Mass Spectrometer. The first chromatographic step utilized a PolyLC HydroxyethylA SEC HPLC column on the following system: G7104A quaternary pump, G7167B autosampler, G7116B column oven at 4° C. and a G7115A diode array detector. The RNA-ligand complex is eluted with phosphate buffered saline solution and captured after elution from the SEC via a six-port valve equipped with a sample loop. After capture in sample loop, the RNA-Ligand complex is introduced into the second chromatography system which consists of a G7120A binary pump, G7116B column oven 45° C., a G7115A diode array detector and equipped with a Waters Xbridge BEH 2.1×50 mm 2.5 µM reverse phase HPLC column utilizing a binary mobile phase of:
A: 0.1% formic acid in water with 2 mM ammonium formate
B: 0.1% formic acid in acetonitrile
Gradient A:B 100:0 is held for 0.8 min; ramp to 1:99 by 1.6 min, is held until 3.1 min; return to 100:0 by 3.3 min. The eluent is diverted to waste from 0 to 0.8 min. The mass spectrometer is tuned and calibrated to manufacturer’s specifications prior to running each plate. Lock masses of 121.0509 Da, and 922.0098 Da m/z are infused during acquisition to calibrate during the run. The SEC column is flushed with watercontaining 50% I from 0.51 to 1.50 minutes during run. Data is analyzed using customized software (Virscidian) and MassHunter software. Binders of interest are detected by exact mass spectrometry. Boron compounds of interest are identified and the assay repeated with each compound at 5 µM and RNA at 0.5-10 µM.

For the K_{D} experiment, boron compounds are dosed at various concentration to make up the final concentration of 0.4-600 µM. RNA concentration is fixed at 0.5-2 µM. All other experiment settings including instrument configuration and analysis parameters remain the same as above. Peak areas of test compounds are extracted using MassHunter software, and plotted and fitted using GraphPad Prism software (SanDiego, CA).

### RNA Fluorescence Polarization (FP) Protection Assay

In the FP assay, fluorophores were added to the 3′ and 5′ end of a short RNA construct and incubated with a boron compound under accelerated degradation conditions (e.g., base and temperature). For intact RNA, the 2 fluorophores come together to produce fluorescence. When the RNA is cleaved, there is no fluorescence. Preservation of fluorescence, therefore, demonstrates RNA protection as gauged vs. control. The counter screen assay run for each boron compound was used as control.

The RNA FP protection assay used a random 30nt RNA oligonucleotide (oligo) with a 5′ Cy5 fluorophore and a 3′ Biotin of the following sequence: 5′-Cy5-

GGAAUCUCUCUCACGAACUGACGUAAUCUU-Biotin-3' (SEQ ID N
O: 9).

In the presence of excess streptavidin, the RNA oligo will bind to the streptavidin and rotational spin will decrease causing an increase in the mP value in a fluorescence polarization (FP) measurement. When the RNA backbone is hydrolyzed under base mediated hydrolysis with increasing pH the Cy5 fluorophore will be free to spin faster lowering the mP value in an FP measurement. Compounds that inhibit base mediated hydrolysis will prevent release of Cy5 and reduction in mP value. A boron compound will have 100% activity when the 30nt RNA oligo remains completely intact, i.e. no hydrolyzation under the basic conditions.

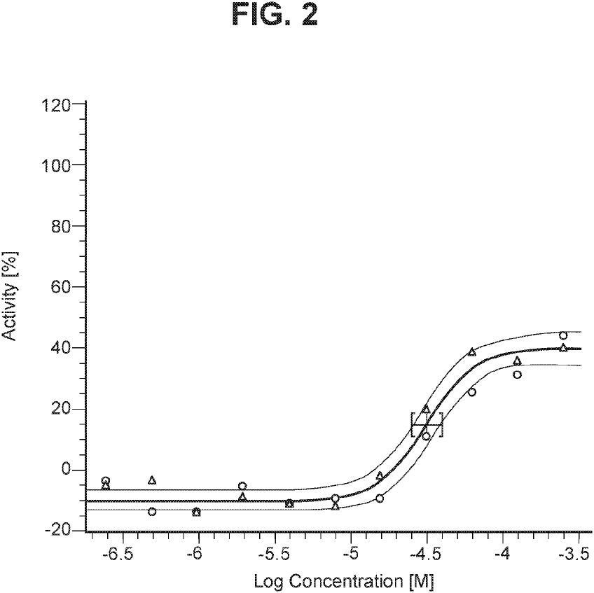

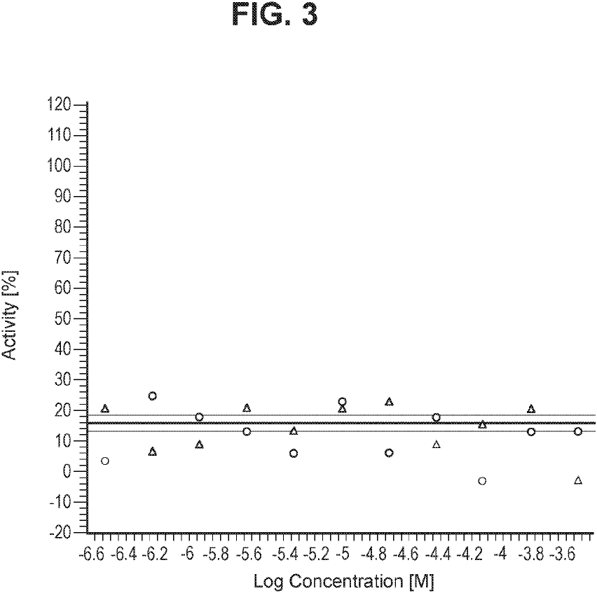

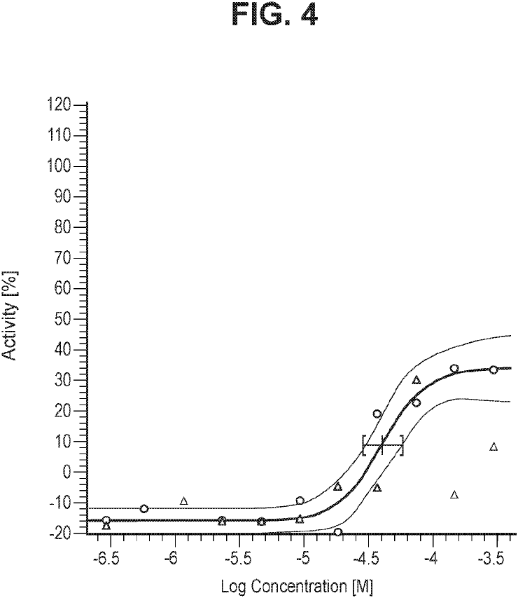

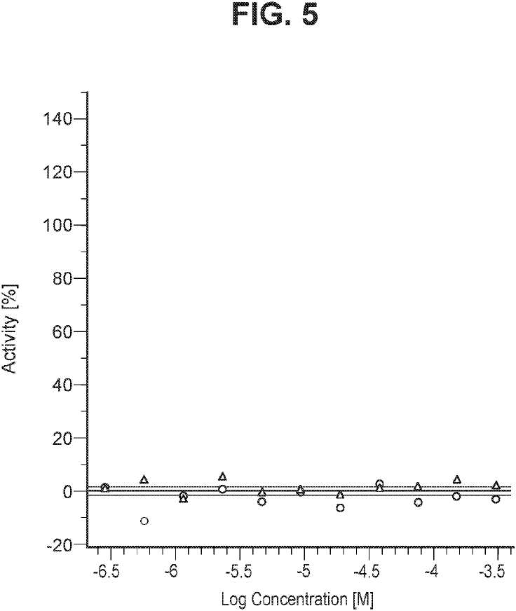

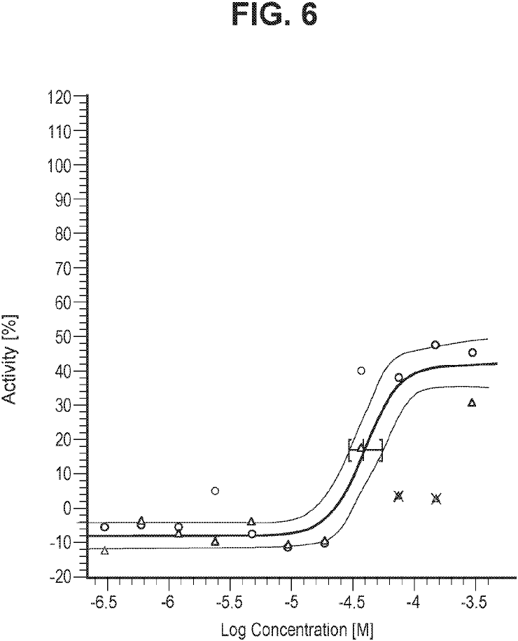

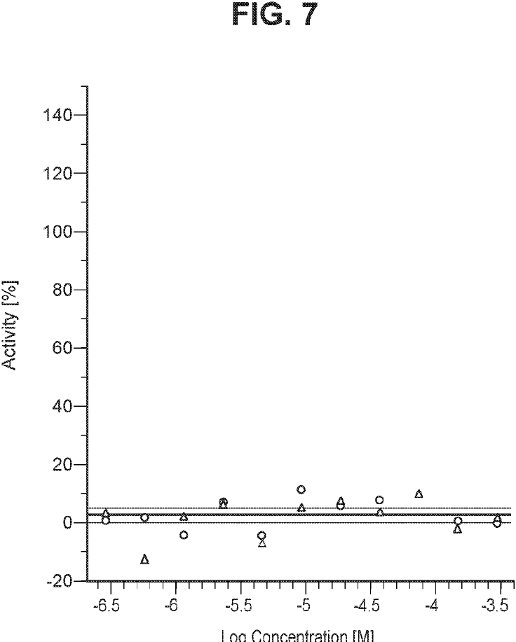

With regard to the plots provided in FIGS. 2-7, the detection for this assay used the fluorescent polarization of the fluorescent dye used in the assay. When a fluorescent dye has a high mass the spin rate is low and when the fluorescence is measured using two polarizing filters one parallel and one perpendicular the amount of spin can be measured. The unit Polarization was measured as mili-Polarization (mP). A dye with high mass will have a high mP reading and low mass will have a low mP reading. Streptavidin was added to bind to the biotin-RNA-Cy5 oligo to achieve high mass and the high mP reading. When the RNA is cleaved by base at high pH the CY5 dye is released from the added streptavidin mass and is free to spin faster resulting in a low mP signal. The y-axis percent activity represents the percent activity of the compound and its ability to protect/stabilize the RNA preventing RNA backbone from being cleaved/degraded. When a boron compound dose provides 100% activity, the RNA is intact/protected and the mP value measured is high. Both a positive control where the RNA was held at pH 7 and not degraded and a negative control with no compound and RNA with 62 mM NaOH were used to allow conversion of the raw mP values of the boron compounds into percent effects for the plots.

To all wells in a compound spotted assay ready plate (Coming #3820) were added 2.5 µL of a 2x Biotin-RNA-Cy5 (Genescript custom #SC1518) and Streptavidin (ThermoFisher#21135) made in nuclease free water, incubated for 20 minutes, 2.5 µL of 50 mM Tris pH 7.2 was added to the positive control wells, and 2.5 µL 125 mM NaOH was added to all other wells. Following incubation for 18 hours, 5 µL 1 M HEPEs pH 7 (Sigma #H3537) was added and the mP was read at Ex/Em/Em of 620/688/688.

Final concentrations in the assay were 40 nM of Biotin-RNA-Cy5, 80 nM Streptavidin in water, with 25 mM Tris buffer pH 7 in control wells, and 62.5 mM NaOH in assay wells. The final compound concentration was 300 µM (single dose) or 600 µM top dose (dose response) with 3% DMSO.

### Syto-9 Dye Intercalation Assay

In the syto-9 dye intercalation assay, boron compounds were pre-incubated with RNA (CH-18 or fgenl) before being subjected to base-mediated hydrolysis. Syto-9 nucleic acid stain, an intercalating dye, was added in neutralizing buffer to stop the base-mediated hydrolysis and determine the % of RNA that remains intact (also known as protective effect of the compound). Fluorescence artifacts were controlled by conducting a counterscreen in parallel with the primary assays. In the counterscreen, the format was the same as the primary assay, except that no RNA was added to any assay wells. The approach involved determining which compounds appeared to generate false positives, i.e. compounds that appeared to protect RNA from base mediated hydrolysis due to compound interference, compound aggregation, compound fluorescence, or other unknown compound interference.

To all wells in a compound spotted assay ready plate (Coming #3820), 4 µL of a 2x RNA made in 100 µM Tris/10 µM EDTA, pH 7 buffer, was added. The reaction was pre-incubated for 30 minutes. Then, 1 µL of 100 µM Tris/10 µM EDTA, pH 7 buffer was added to positive control wells. Then, 1 µL 5X NaOH was added to all other wells. These were incubated for 24 hours at room temperature. Following incubation, 4 µL 2X Syto-9 Nucleic acid stain in 1 M HEPES (neutralizing buffer) at pH 7 was added with resulting samples analyzed using fluorescence intensity at Excitation and Emission of 485 and 520 nm, respectively. Final concentrations in the assay were 100 nM fgenl RNA or 30 nM Chi-18 RNA in 100 µM Tris/10 µM EDTA, pH 7 buffer with 100 µM Tris/10 µM EDTA, pH 7 in control wells and 60 mM NaOH in assay wells. Neutralizing buffer final concentration was 5 µM Syto-9 in 1 M HEPES, pH 7. Final compound concentration was 300 µM with 3% DMSO.

For the counterscreen, to all wells in a compound spotted assay ready plate (Coming #3820) 4 µL of a 100 µM Tris/10 µM EDTA, pH 7 buffer was added. Then, 1 µL of 2X RNA in 100 µM Tris/10 µM EDTA, pH 7 buffer was added to positive control wells. Following this, 1 µL 5X NaOH was added to all other assay wells. These were incubated for 24 hours at room temperature. From there, 4 µL 2X Syto-9 Nucleic acid stain in 1 M HEPES (neutralizing buffer), pH 7 was added with resulting samples analyzed using fluorescence intensity at Excitation and Emission of 485 and 520 nm, respectively. Final concentrations in the assay are 300 µM (single point or top concentration dose response) compound in 100 µM Tris/10 µM EDTA, pH 7 buffer with 100 nM fgenl or 30 nM Chi-18 RNA in control wells and 60 mM NaOH in assay wells. Neutralizing buffer final concentration was 5 µM Syto-9 in 1 M HEPES at pH 7.

### TapeStation Assay

The Agilent 4150 TapeStation bioanalyzer is an automated platform for electrophoresis of nucleic acids. In this case, the system was used to measure the integrity of RNA samples after various degradation conditions and show protection from degradation by compounds. In principle, samples are loaded to the instrument where electrophoresis separates component RNA’s on the basis of mass, and subsequently analyzes these optically as they travel through the capillary.

Model Chi18 or Fgenl RNAs were diluted to 50 ng/µL in 50 mM Tris buffer pH 11.4. Compound (30 mM stock in DMSO) or DMSO alone was added to yield final concentrations ranging from 200 mM to 1.2 mM. The DMSO concentration was adjusted to remain constant between all samples (max 4%), and the samples were aliquoted to PCR tubes for each time point.

Samples were prepared for the TapeStation according to the manufacturer’s recommendations for the standard sensitivity RNA ScreenTape [ sample + 5 µL RNA sample buffer; heated at 73° C. for 3 min, chilled on ice 2 min] and analyzed on the Agilent 4150 TapeStation using the RNA ScreenTape Assay protocol. Resulting electropherograms were compared, and a region representing the intact RNA (i.e. 500-100 nt for Fgenl RNA [612nt]) was sectioned using the software to determine the concentration of the intact fragment.

### Native Electrospray Ionization (nESI) Mass Spectrometry (nESI-MS)

Mass spectrometry is rapidly expanding its role in biophysics and structural biology due to its high sensitivity, throughput, accuracy, and resolution. With orders of magnitude less sample required compared to traditional methods, detailed structural information can be extracted for biomolecules in either purified form or from complex mixtures. In particular, nESI-MS has been demonstrated as a powerful complimentary tool for the characterization of target-binder interactions, and it has been increasingly utilized to derive detailed structural and mechanistic information for target-binder interactions including discerning stoichiometry, binding strength, and in some cases, binding sites. In combination with the traditional LC-MS, nESI-MS can be applied to differentiate covalent or reversable covalent interaction from non-covalent interaction.

The RNA samples are first buffer exchanged into 200 mM ammonium acetate buffer in DEPC water at pH 7.4 using a Micro Bio-Spin 6 column from BioRad (Hercules, CA). The RNA sample is then mixed with potential binders/stabilizers at a typical final concentration ratio of ⅒ µM with DMSO at or below 1% for 1 hour incubation at ambient room temperature prior to nESI-MS analysis. nESI-MS measurements are carried out utilizing the Thermo Exactive Plus EMR Orbitrap MS equipped with a Nanospray Flex Ion Source (Thermo Fisher Scientific, Bremen, Germany). A 3 µL sample is loaded into an offline electrospray capillary (GlassTip 4 µm i.d.; New Objective, Wobum, MA), and high voltage 1.4-2.0 kV is applied to start and maintain a stable nanoelectrospray. The EMR MS is operated in the extended mass range (EMR) mode with critical parameters tuned to preserve the RNA/stabilizer complex and meanwhile, to maintain decent MS detection sensitivity (e.g., sufficient ionization). Some critical parameters utilized for high quality MS measurement include source temperature held at 150° C., in-source CID 20 V, CE 20 V, and trapping gas 2. A few other important parameters are as follows: source DC offset 25 V, injection flatapole DC 5 V, inter flatapole lens 7 V, and bent flatapole DC 7 V. A typical scan range is set with 240-10000 Da m/z to be able to detect the RNA/stabilizer complex, RNA alone and the stabilizers themselves. Mass spectra are recorded with resolution either at 8k/17k (at m/z 200 Da) for average masses or at 70k/140k Da for isotopic masses in the case of smaller RNA molecules. The instrument is externally calibrated using Csl solution with up to m/z 11034 Da, and the data processing is performed using Thermo Xcalibur 4.1 Qual Browser and Intact Mass 3.9 from Protein Metrics Inc.

### Nuclear Magnetic Resonance (NMR) Spectroscopy

NMR has evolved into a powerful tool for a broad range of applications in early phase drug discovery where it is often regarded as the “gold-standard” to identify and characterize weak inter-molecular interactions. Ligand- and target detected NMR methods can provide binding, dynamic and structural data to inform biological questions on target and ligand integrity, target-ligand interaction sites, mechanism of action as well as the enablement of Structure Based Drug Discovery (SBDD).

The linewidths of ligand ¹H-NMR signals are sensitive to target binding, and a ligand-detected approach was therefore employed where the line-broadening of the compound signals in presence of the RNA was used as indicator for target engagement. NMR samples typically may contain 75 µM RNA, 150 µM compound in 50 mM K phosphate, 50 mM NaCl, 10% (v/v) D₂O at pH 6.5. All NMR experiments are carried out at 298 K on a Bruker AVANCE spectrometer operating at a ¹H-Larmor frequency of 600 MHz equipped with a 1.7 mm TCI micro-cryoprobe and a SampleJet automated sample handler (Bruker Billerica, MA). 1D ¹H-NMR spectra are recorded with a data size of 2048 complex points, an acquisition time of 113 ms and 1024 scans. The WATERGATE pulse train W5 is used for water suppression, with the W5 window delay being adjusted to 150 µs. All NMR spectra are processed using Topspin 3.5pl7. Before Fourier transformation, the data vectors are zero-filled and multiplied with 75°-shifted sine-bell function. Chemical shifts are referenced to the residual solvent signal.

### RNA Sequences

Fgenl and Chi18-4 model RNA were chosen and designed based on Leija-Martinex et al., with 5′ and 3′ primer annealing sites at the two ends (N. Leija-Martinez et al., The separation between the 5′-3′ ends in long RNA molecules is short and nearly constant. Nucleic Acids Res 42, 13963-13968 (2014)).

The Fgenl RNA sequences were derived from a fungal organism named Trichoderma atroviride and are available on the fungal genomics resource database with protein ID 258498.

> RNA sequence derived from HCV IRES
GCCAGCCCCCGAUUGGGGGCGACACUCCACCAUAGAUCACUCCCCUGUGAGG AACUACUGUCUUCACGCAGAAAGCGUCUAGCCAUGGCGUUAGUAUGAGUGUC GUGCAGCCUCCAGGACCCCCCCUCCCGGGAGAGCCAUAGUGGUCUGCGGAA CCGGUGAGUACACCGGAAUUGCCAGGACGACCGGGUCCUUUCUUGGAUCAAC CCGCUCAAUGCCUGGAGAUUUGGGCGUGCCCCCGCGAGACUGCUAGCCGaAG UAGUGUUGGGUCGCGAAAGGCCUUGUGGUACUGCCUGAUAGGGUGCUUGCG AGUGCCCCGGGAGGUCUCGUAGACCGUGCACCAUGAGCACGAAUCCUAAACC UCAAAGAAAAACCAAACGUAACACCAACCGCCGCCCACAGGACGUCUGAGGCG GCCGCCGCCUCAGACGUCcuGUGGGUUUAUUGCAUCCCGC (SEQ ID NO: 1)
> DNA transcript, derived from HCV IRES (T7 promoter in bold; site in italics) 5′-
GGATCCTAATACGACTCACTATAGCCAGCCCCCGATTGGGGGCGACACTCCAC CATAGATCACTCCCCTGTGAGGAACTACTGTCTTCACGCAGAAAGCGTCTAGCC ATGGCGTTAGTATGAGTGTCGTGCAGCCTCCAGGACCCCCCCTCCCGGGAGAG CCATAGTGGTCTGCGGAACCGGTGAGTACACCGGAATTGCCAGGACGACCGGG TCCTTTCTTGGATCAACCCGCTCAATGCCTGGAGATTTGGGCGTGCCCCCGCGA GACTGCTAGCCGAGTAGTGTTGGGTCGCGAAAGGCCTTGTGGTACTGCCTGAT AGGGTGCTTGCGAGTGCCCCGGGAGGTCTCGTAGACCGTGCACCATGAGCACG AATCCTAAACCTCAAAGAAAAACCAAACGTAACACCAACCGCCGCCCACAGGAC GTCTGAGGCGGCCGCCGCCTCAGACGTCCTGTGGGTTTATTGCATCCCGCCTC GAGTCTAGA-3′ (SEQ ID NO: 2)
> Fgenl model RNA synthesized RNA sequence
UCUCUCGCUAUCUCGGAAUCGAGGGGUCUGGCCUACGCUGCGAUUGCUUCU UCAGAGCAUCCUUCUUCAGCCUUGUGCCCUCUACAGUGGCAGCCUCACACAA ACUAUCAACAUGGCUUCCGAAUCCAGGCUCUACCAGAUCUCCGGCGAGACCA AGUCUCAUCUGCUCAAGUUCCGCUUGACGACAUCUCGGGCCAGCAAACCUCA GGCAGUGAUUUAUUUGAUUGAUAAAAACACUCACGAAAUCCGCCAAGACGAUG ACAAGACUGUAUACACCUCCCUCGACGAGAUAGCCGACGACCUCCCCGACAG CACCCCUCGAUUCAUCUUCCUCAGCUAUCCCUUGACGAUGCCCGAUGGACGG CUAUCCGUGCCUUACACCAUGAUCUACUACCUCCCCAUCAACUGCAAUGCCG CAACAAGGAUGCUCUACGCCGGCGCAAAAGAGCUGAUACGCAACACGGCCGA GGUGAACAAGGUUAUCGAUAUAGAGUCUGCAGAGGAUCUGGAAGAUAUUCCA AAGCAGUUGAGCGGAUAGAAAUAAUAAUAAAAGAAAUAACAUGUUCGCUAUGC C (SEQ ID NO: 3)
> Fgenl model RNA synthesized RNA sequence, with primer annealing sites
UCCACCAUGAAUCACUCCUCUCUCGCUAUCUCGGAAUCGAGGGGUCUGGCC UACGCUGCGAUUGCUUCUUCAGAGCAUCCUUCUUCAGCCUUGUGCCCUCUAC AGUGGCAGCCUCACACAAACUAUCAACAUGGCUUCCGAAUCCAGGCUCUACCA GAUCUCCGGCGAGACCAAGUCUCAUCUGCUCAAGUUCCGCUUGACGACAUCU CGGGCCAGCAAACCUCAGGCAGUGAUUUAUUUGAUUGAUAAAAACACUCACGA AAUCCGCCAAGACGAUGACAAGACUGUAUACACCUCCCUCGACGAGAUAGCC GACGACCUCCCCGACAGCACCCCUCGAUUCAUCUUCCUCAGCUAUCCCUUGA CGAUGCCCGAUGGACGGCUAUCCGUGCCUUACACCAUGAUCUACUACCUCCC CAUCAACUGCAAUGCCGCAACAAGGAUGCUCUACGCCGGCGCAAAAGAGCUG AUACGCAACACGGCCGAGGUGAACAAGGUUAUCGAUAUAGAGUCUGCAGAGG AUCUGGAAGAUAUUCCAAAGCAGUUGAGCGGAUAGAAAUAAUAAUAAAAGAAA UAACAUGUUCGCUAUGCCGAAAAACCAAACGUAACACC (SEQ ID NO: 4)
> DNA template of Fgenl model RNA, with primer annealing sites
TCCACCATGAATCACTCCTCTCTCGCTATCTCGGAATCGAGGGGTCTGGCCTAC GCTGCGATTGCTTCTTCAGAGCATCCTTCTTCAGCCTTGTGCCCTCTACAGTGGC AGCCTCACACAAACTATCAACATGGCTTCCGAATCCAGGCTCTACCAGATCTCCG GCGAGACCAAGTCTCATCTGCTCAAGTTCCGCTTGACGACATCTCGGGCCAGCA AACCTCAGGCAGTGATTTATTTGATTGATAAAAACACTCACGAAATCCGCCAAGAC GATGACAAGACTGTATACACCTCCCTCGACGAGATAGCCGACGACCTCCCCGAC AGCACCCCTCGATTCATCTTCCTCAGCTATCCCTTGACGATGCCCGATGGACGGC TATCCGTGCCTTACACCATGATCTACTACCTCCCCATCAACTGCAATGCCGCAACA AGGATGCTCTACGCCGGCGCAAAAGAGCTGATACGCAACACGGCCGAGGTGAA CAAGGTTATCGATATAGAGTCTGCAGAGGATCTGGAAGATATTCCAAAGCAGTTGA GCGGATAGAAATAATAATAAAAGAAATAACATGTTCGCTATGCCGAAAAACCAAAC GTAACACC (SEQ ID NO: 5)
> Chi18-4 model RNA synthesized RNA sequence
UACGCCAGACAAUAGAUUCAGCUCGAAUAAUGAAGCCGAUUGGUCACUGAUUA ACUUGGUGUAUCUACUGGGUAGCAAAGGACCGAUCUUCACCUCGCAUGACCG UUGAAUGUGGCGCAAUUCUAGUACUUGAAUGGCCUGGCCAAGCUUAUAGAAC AAUCGAGACGAAUUGAACACCACUAUCAAAAUCCAAAUUAUGCGGUUUUCAAU CAUACAUGUGGCUUUAUGGCUGGCCAUGGCGGCCGGUUCAUUUACAGCUAAC GCUUACGGAGCUGUUCGAUGCGUCAUGUAUCUCACAGGGCAACACGUGGUAG UCCCUUCAGAACCUCAUCUCGUGGAUUCCAUAACCCAUUUGAUACUGGCUUU UAUGCGCUCUGAUGUCUUCAAUGUGGACAAAACGCCUGCUGAAUUCCCGCUC UUCGCAUCAGUUGCUGAGACGCGCGAGAAGUUCAACGCAAAUACCAAGAUCA UGGUCGCAAUCGGCGGUUGGGGAGACGCAGGAUUUGAAGAAGCUGCGCGUG ACGAUUCAUCGAGAAAGCGGUGGGCUGGCCACGUCAAGGCCAUGGUUGACCA GACAGGAGCCGAUGGCAUUGACAUUGACUGGGAAUAUCCGGGAGGGAACCGU GACGAUUAUAAACUCAUUCCGAACUCUCAGCGGGAAUGGGAGAUUCAGGCAU UCGUGCUCCUUCUUCAAGAAUUGCGUUCCGCCUUGGGACAAACGAAACUGCU CUCAAUUGCGGUGCCAGCGCUGGAACGAGAUUUGAUGGCCUUCACAAACUCG ACUAUUCCGUCCAUUGUGGAUCAAGUUGACUUUAUCAGUGUAAUGACUUACG ACAUGAUGAACCGACGCGACAACGUUGUCAAGCACCACAGCGGCGUGGCUGA CUCCUGGGAAGCAAUGAAGCGAUAUAUAGAUCGCGGCGUCCCUCCGCACAAG CUGAACUUUGGACUUGGUUACUAUGCCAAAUGGUUCAUGACUGAACAAUGCG AUGUACAGCACCCAUUAGGAUGCCGCACUCAACUGCUAGAAGACCCCGCCAA UGGAGCCGAUCUUGGCAAGACUGCAGCUUUUAGCUGGCACGACGAGGUUCCC GCAGAAAUGGCCAAUUCCUUUGGCAAAGCCCAUGCUCAUGGCCGCUACUAUG AAGAUGGAAGCUACGGCUAUUGGGACGAUGAAGAGAAGAGAUGGUGGUCCUA CGACACGCCUCUCACCAUCAAAGCCAAAGUGCCUCGGCUUCUCGGCGAGCUG CAGCUGGGCGGGGUGUUUGCCUGGGGGCUGGGCGAGGACGCUCCGCAGUU UCUGCACCUGAAGGCGACUGUUGACGGCAUUCGGGUUCUGCGCGGAGAUGA CGAUGCUGUGAAGGAUGAGCUGUAACAAGCAGCGGCGUGUGUACGUCUGGA GUUUGGCGACAUCAUUGAAGGCGUGGGAUGCAAUUCAAGCCAUUGAUACUGC AGAUAAGUUGAAGCAGCAACAGCCAAAACUCUGCACGUGGCCCGAGACUUCC GUCUUAUAGCAGGGAACAGACAGGAUGGUGCUGACGGAAUAUGCAGAGCUCG AGCUCAACCGGUAGGCUAGCUUGAAAACCCCAUGAGCUAGCAGUUAACCUUG CAGUUAAGGGUAACCAGAGCUUAAACACGUCUGAUUUGGUGCAAUGAAUACA GGCUGC (SEQ ID NO: 6)
> Chi18-4 model RNA synthesized RNA sequence, primer annealing sites in bold
UCCACCAUGAAUCACUCCUACGCCAGACAAUAGAUUCAGCUCGAAUAAUGAA GCCGAUUGGUCACUGAUUAACUUGGUGUAUCUACUGGGUAGCAAAGGACCGA UCUUCACCUCGCAUGACCGUUGAAUGUGGCGCAAUUCUAGUACUUGAAUGGC CUGGCCAAGCUUAUAGAACAAUCGAGACGAAUUGAACACCACUAUCAAAAUCC AAAUUAUGCGGUUUUCAAUCAUACAUGUGGCUUUAUGGCUGGCCAUGGCGGC CGGUUCAUUUACAGCUAACGCUUACGGAGCUGUUCGAUGCGUCAUGUAUCUC ACAGGGCAACACGUGGUAGUCCCUUCAGAACCUCAUCUCGUGGAUUCCAUAA CCCAUUUGAUACUGGCUUUUAUGCGCUCUGAUGUCUUCAAUGUGGACAAAAC GCCUGCUGAAUUCCCGCUCUUCGCAUCAGUUGCUGAGACGCGCGAGAAGUUC AACGCAAAUACCAAGAUCAUGGUCGCAAUCGGCGGUUGGGGAGACGCAGGAU UUGAAGAAGCUGCGCGUGACGAUUCAUCGAGAAAGCGGUGGGCUGGCCACG UCAAGGCCAUGGUUGACCAGACAGGAGCCGAUGGCAUUGACAUUGACUGGGA AUAUCCGGGAGGGAACCGUGACGAUUAUAAACUCAUUCCGAACUCUCAGCGG GAAUGGGAGAUUCAGGCAUUCGUGCUCCUUCUUCAAGAAUUGCGUUCCGCCU UGGGACAAACGAAACUGCUCUCAAUUGCGGUGCCAGCGCUGGAACGAGAUUU GAUGGCCUUCACAAACUCGACUAUUCCGUCCAUUGUGGAUCAAGUUGACUUU AUCAGUGUAAUGACUUACGACAUGAUGAACCGACGCGACAACGUUGUCAAGC ACCACAGCGGCGUGGCUGACUCCUGGGAAGCAAUGAAGCGAUAUAUAGAUCG CGGCGUCCCUCCGCACAAGCUGAACUUUGGACUUGGUUACUAUGCCAAAUGG UUCAUGACUGAACAAUGCGAUGUACAGCACCCAUUAGGAUGCCGCACUCAAC UGCUAGAAGACCCCGCCAAUGGAGCCGAUCUUGGCAAGACUGCAGCUUUUAG CUGGCACGACGAGGUUCCCGCAGAAAUGGCCAAUUCCUUUGGCAAAGCCCAU GCUCAUGGCCGCUACUAUGAAGAUGGAAGCUACGGCUAUUGGGACGAUGAAG AGAAGAGAUGGUGGUCCUACGACACGCCUCUCACCAUCAAAGCCAAAGUGCC UCGGCUUCUCGGCGAGCUGCAGCUGGGCGGGGUGUUUGCCUGGGGGCUGG GCGAGGACGCUCCGCAGUUUCUGCACCUGAAGGCGACUGUUGACGGCAUUC GGGUUCUGCGCGGAGAUGACGAUGCUGUGAAGGAUGAGCUGUAACAAGCAG CGGCGUGUGUACGUCUGGAGUUUGGCGACAUCAUUGAAGGCGUGGGAUGCA AUUCAAGCCAUUGAUACUGCAGAUAAGUUGAAGCAGCAACAGCCAAAACUCUG CACGUGGCCCGAGACUUCCGUCUUAUAGCAGGGAACAGACAGGAUGGUGCUG ACGGAAUAUGCAGAGCUCGAGCUCAACCGGUAGGCUAGCUUGAAAACCCCAU GAGCUAGCAGUUAACCUUGCAGUUAAGGGUAACCAGAGCUUAAACACGUCUG AUUUGGUGCAAUGAAUACAGGCUGCGAAAAACCAAACGUAACACC (SEQ ID NO: 7)
> DNA transcript for Chi18-4 model RNA synthesized RNA sequence, primer annealing sites in bold
TCCACCATGAATCACTCCTACGCCAGACAATAGATTCAGCTCGAATAATGAAGCC GATTGGTCACTGATTAACTTGGTGTATCTACTGGGTAGCAAAGGACCGATCTTCA CCTCGCATGACCGTTGAATGTGGCGCAATTCTAGTACTTGAATGGCCTGGCCAAG CTTATAGAACAATCGAGACGAATTGAACACCACTATCAAAATCCAAATTATGCGGT TTTCAATCATACATGTGGCTTTATGGCTGGCCATGGCGGCCGGTTCATTTACAGCT AACGCTTACGGAGCTGTTCGATGCGTCATGTATCTCACAGGGCAACACGTGGTA GTCCCTTCAGAACCTCATCTCGTGGATTCCATAACCCATTTGATACTGGCTTTTAT GCGCTCTGATGTCTTCAATGTGGACAAAACGCCTGCTGAATTCCCGCTCTTCGCA TCAGTTGCTGAGACGCGCGAGAAGTTCAACGCAAATACCAAGATCATGGTCGCA ATCGGCGGTTGGGGAGACGCAGGATTTGAAGAAGCTGCGCGTGACGATTCATC GAGAAAGCGGTGGGCTGGCCACGTCAAGGCCATGGTTGACCAGACAGGAGCC GATGGCATTGACATTGACTGGGAATATCCGGGAGGGAACCGTGACGATTATAAAC TCATTCCGAACTCTCAGCGGGAATGGGAGATTCAGGCATTCGTGCTCCTTCTTCA AGAATTGCGTTCCGCCTTGGGACAAACGAAACTGCTCTCAATTGCGGTGCCAGC GCTGGAACGAGATTTGATGGCCTTCACAAACTCGACTATTCCGTCCATTGTGGAT CAAGTTGACTTTATCAGTGTAATGACTTACGACATGATGAACCGACGCGACAACG TTGTCAAGCACCACAGCGGCGTGGCTGACTCCTGGGAAGCAATGAAGCGATATA TAGATCGCGGCGTCCCTCCGCACAAGCTGAACTTTGGACTTGGTTACTATGCCAA ATGGTTCATGACTGAACAATGCGATGTACAGCACCCATTAGGATGCCGCACTCAA CTGCTAGAAGACCCCGCCAATGGAGCCGATCTTGGCAAGACTGCAGCTTTTAGC TGGCACGACGAGGTTCCCGCAGAAATGGCCAATTCCTTTGGCAAAGCCCATGCT CATGGCCGCTACTATGAAGATGGAAGCTACGGCTATTGGGACGATGAAGAGAAG AGATGGTGGTCCTACGACACGCCTCTCACCATCAAAGCCAAAGTGCCTCGGCTT CTCGGCGAGCTGCAGCTGGGCGGGGTGTTTGCCTGGGGGCTGGGCGAGGACG CTCCGCAGTTTCTGCACCTGAAGGCGACTGTTGACGGCATTCGGGTTCTGCGCG GAGATGACGATGCTGTGAAGGATGAGCTGTAACAAGCAGCGGCGTGTGTACGTC TGGAGTTTGGCGACATCATTGAAGGCGTGGGATGCAATTCAAGCCATTGATACTG CAGATAAGTTGAAGCAGCAACAGCCAAAACTCTGCACGTGGCCCGAGACTTCCG TCTTATAGCAGGGAACAGACAGGATGGTGCTGACGGAATATGCAGAGCTCGAGC TCAACCGGTAGGCTAGCTTGAAAACCCCATGAGCTAGCAGTTAACCTTGCAGTTA AGGGTAACCAGAGCTTAAACACGTCTGATTTGGTGCAATGAATACAGGCTGCGAA AAACCAAACGTAACACC (SEQ ID NO: 8)
> RNA FP Stabilization Assay
GGAAUCUCUCUCACGAACUGACGUAAUCUU (SEQ ID NO: 9)

We claim:

1. A method of protecting RNA from degradation by contacting the RNA with a boron compound.

2. The method according to claim 1 wherein the RNA is single strand.

3. The method according to claim 1 wherein the RNA is antisense single strand.

4. The method according to claim 1 wherein the RNA is messenger RNA (mRNA).

5. The method according to claim 1 wherein the RNA encodes a viral antigen.

6. The method according to claim 1, wherein the boron compound is selected from the group consisting of:
N-[(5aR,6aS,7S,10aS)-9-carbamoyl-4,7-bis(dimethylamino)-1,8,10a,11-tetrahydroxy-10,12-dioxo-5,5a,6,6a,7,10,10a,12-octahydrotetracen-2-yl]-1-hydroxy-3,3-dimethyl-1,3-dihydro-2,1-benzoxaborole-6-carboxamide;
(4S,4aR,5S,5aR,6R,12aS)-4-(dimethylamino)-3,5,10,12,12a-pentahydroxy-9-({2-[(1-hydroxy-1,3-dihydro-2,1-benzoxaborol-6-yl)amino]-2-oxoethyl}amino)-6-methyl-1,11-dioxo-1,4,4a,5,5a,6,11,12a-octahydrotetracene-2-carboxamide;
N~1~-[(5R,5aR,6S,6aR,7S,10aS)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-N~4~-(1-hydroxy-1,3-dihydro-2,1-benzoxaborol-6-yl)butanediamide;
(4S,4aR,5S,5aR,6R,12aS)-4-(dimethylamino)-3,5,10,12,12a-pentahydroxy-9-({3-[(1-hydroxy-3,4-dihydro-1H-2,5,1-benzodioxaborepin-7-yl)amino]-3-oxopropyl}amino)-6-methyl-1,11-dioxo-1,4,4a,5,5a,6,11,12a-octahydrotetracene-2-carboxamide;
(4S,4aR,5S,5aR,6R,12aS)-9-({3-[(2,3-dihydro-7H-[1,2]oxaborolo[4,3,2-jk][2,5,1]benzodioxaborepin-9-yl)amino]-3-oxopropyl}amino)-4-(dimethylamino)-3,5,10,12,12a-pentahydroxy-6-methyl-1,11-dioxo-1,4,4a,5,5a,6,11,12a-octahydrotetracene-2-carboxamide;
quinolin-8-yl {3-chloro-4-[(dimethylamino)methyl]phenyl}(3-cyanophenyl)borinate; (4-fluorophenyl){3-[(4-methylpiperazin-1-yl)methyl]phenyl}(quinolin-8-olato-kappa~2~N,O)boron;
N-[(5R,5aR,6S,6aR,7S,10aS)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-1-hydroxy-3,3-dimethyl-1,3dihydro-2,1-benzoxaborole-6-carboxamide;
(4S,4aR,5S,5aR,6R,12aS)-4-(dimethylamino)-3,5,10,12,12a-pentahydroxy-9-({3-[(1-hydroxy-3,4-dihydro-1H-2,1-benzoxaborinin-7-yl)amino]-3-oxopropyl}amino)-6-methyl-1,11-dioxo-1,4,4a,5,5a,6,11,12a-octahydrotetracene-2-carboxamide;
quinolin-8-yl (3-chloro-4-cyanophenyl){3-fluoro-4-[(4-methylpiperazin-1-yl)methyl]phenyl}borinate;
N~1~-[(5RS,5aRS,6SR,6aRS,7SR,10aSR)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-N~4~-{[(5RS)-2-hydroxy-1,2-oxaborolan-5-yl]methyl}butanediamide;
{3-chloro-4-[(4-methylpiperazin-1-yl)methyl]phenyl}(4-cyanophenyl)(quinolin-8-olato-kappa~2~N,O)boron;
dimethyl 4-(4,4,5,5-tetramethyl-1,3,2-dioxaborolan-2-yl)pyridine-2,6-dicarboxylate;
[(3S)-4-borono-1-hydroxy-1,3-dihydro-2,1-benzoxaborol-3-yl]acetic acid;
(4S,4aR,5S,5aR,6R,12aS)-4-(dimethylamino)-3,5,10,12,12a-pentahydroxy-9-{[N-(1-hydroxy-3,3-dimethyl-1,3-dihydro-2,1-benzoxaborol-6-yl)glycyl]amino}-6-methyl-1,11-dioxo-1,4,4a,5,5a,6,11,12a-octahydrotetracene-2-carboxamide;
quinolin-8-yl (3-chloro-4-cyanophenyl){4-[(4-methylpiperazin-1-yl)methyl]phenyl}borinate;
quinolin-8-yl (4-cyanophenyl){3-[(4-methylpiperazin-1-yl)methyl]phenyl}borinate;
N-[(6aS,10S,10aR,11S,11aR,12R)-8-carbamoyl-10-(dimethylamino)-4,6,6a,9,11-pentahydroxy-12-methyl-5,7-dioxo-5,6a,7,10,10a,11,11a,12-octahydrotetracen-1-yl]-1-hydroxy-3,3-dimethyl-1,3-dihydro-2,1-benzoxaborole-6-carboxamide;
N-(1-hydroxy-1,3-dihydro-2,1-benzoxaborol-6-yl)benzamide;
bis(acetato-kappaO)[1-cyclopropyl-6,7-difluoro-8-methyl-4-(oxo-kappaO)-1,4-dihydroquinoline-3-carboxylato-kappaO]boron;
N-[(6aS,10S,10aR,11S,11aR,12R)-8-carbamoyl-10-(dimethylamino)-4,6,6a,9,11-pentahydroxy-12-methyl-5,7-dioxo-5,6a,7,10,10a,11,11a,12-octahydrotetracen-1-yl]-1-hydroxy-1,3-dihydro-2,1-benzoxaborole-6-carboxamide;
N-(3-{[(5aR,6aS,7S,10aS)-9-carbamoyl-4,7-bis(dimethylamino)-1,8,10a,11-tetrahydroxy-10,12-dioxo-5,5a,6,6a,7,10,10a,12-octahydrotetracen-2-yl]amino}-3-oxopropyl)-1-hydroxy-1,3-dihydro-2,1-benzoxaborole-5-carboxamide;
quinolin-8-yl [3-(4,4-dimethyl-4,5-dihydro-1,3-oxazol-2-yl)phenyl](3-fluorophenyl)borinate;
{3-[(tert-butoxycarbonyl)amino]propyl}(trifluorido)borate(1-);
{3-[benzyl(methyl)amino]prop-1-en-2-yl}(trifluorido)borate(1-);
{(2Z)-3-(dimethylamino)-3-hydroxy-1-[4-hydroxy-2-(hydroxy-kappaO)phenyl]prop-2-en-1-onato-kappaO}(difluorido)boron;
5-phenoxy-2,1-benzoxaborol-1(3H)-ol;
bis(4-chlorophenyl)borinic acid;
(4-{[(3R)-1-(tert-butoxycarbonyl)piperidin-3-yl](3-methylpyridin-2-yl)carbamoyl}phenyl)(trifluorido)borate(1-);
N-[(3-methyl-1,2-oxazol-5-yl)methyl]-2-[(3S)-3-(2-methylphenyl)piperidin-1-yl]-7H-pyrrolo[2,3-d]pyrimidin-4-amine;
N-[(5R,5aR,6S,6aR,7S,10aS)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-1-hydroxy-1,3-dihydro-2,1-benzoxaborole-6-carboxamide;
[(1Z)-1-(dimethylamino)-1-hydroxy-5-(hydroxy-kappaO)-4-phenylpenta-1,4-dien-3-onato-kappaO](difluorido)boron;
[3-(5-fluoro-2,1-benzoxaborol-1(3H)-yl)phenyl]methyl8-hydroxyquinoline-2-carboxylate;
2-({[bis(3-fluorophenyl)boranyl]oxy}carbonyl)pyridin-3-ol;
3-methyl-4-(4,4,5,5-tetramethyl-1,3,2-dioxaborolan-2-yl)benzoicacid;
2-(4-bromophenyl)-5,6-dichloro-2H-1,3,2-benzodioxaborole;and
4,4′-{[1-(nonaboran-1-yl)-1H-borirene-2,3-diyl]bis(methylene)}diphenol, or a pharmaceutically acceptable salt thereof.

7. A composition comprising an RNA and at least one boron compound.

8. The composition according to claim 7, wherein the at least one boron compound is selected from the group consisting of:
N-(1-hydroxy-1,3-dihydrobenzo[c][1,2]oxaborol-6-yl)benzamide;
2,2′-(cyclohexane-1,1-diyl)bis(4,4,5,5-tetramethyl-1,3,2-dioxaborolane);
5-(2-(aminomethyl)-4-(trifluoromethyl)phenoxy)benzo[c][1,2]oxaborol-1(3H)-ol;
1-hydroxy-3,3-dimethyl-1,3-dihydrobenzo[c][1,2]oxaborole-6-carboxylic acid;
N-(3-(1-hydroxy-1,3-dihydrobenzo[c][1,2]oxaborol-4-yl)phenyl)-5-(1-methyl-1H-benzo[d]imidazol-2-yl)pentanamide;
4-(5-(3-chlorophenyl)-5-(trifluoromethyl)-4,5-dihydroisoxazol-3-yl)-N-(1-hydroxy-2-methyl-1,2-dihydrobenzo[d][1,2,3]diazaborinin-6-yl)-2-methylbenzamide;
3-(4-fluorobenzyl)-5-(4-(((1-hydroxy-1,3-dihydrobenzo[c][1,2]oxaborol-6-yl)oxy)methyl)benzylidene)thiazolidine-2,4-dione;
4-(5-(3,5-dichloro-4-fluorophenyl)-5-(trifluoromethyl)-4,5-dihydroisoxazol-3-yl)-N-((1-hydroxy-3,3-dimethyl-1,3-dihydrobenzo[c][1,2]oxaborol-5-yl)methyl)-2-methylbenzamide;
2-(6-((2-((2-aminoethyl)amino)pyrimidin-4-yl)oxy)-1-hydroxy-4-methyl-1,3-dihydrobenzo[c][1,2]oxaborol-3-yl)acetic acid;
5-phenoxybenzo[c][1,2]oxaborol-1(3H)-ol;
5-(pyridin-2-ylmethoxy)benzo[c][1,2]oxaborol-1(3H)-ol;
8-(3-aminopropoxy)benzo[d][1,2,3]diazaborinin-1(2H)-ol;
4-(aminomethyl)-N-(1-hydroxy-1,3-dihydrobenzo[c][1,2]oxaborol-6-yl)benzenesulfonamide;
N-((1R,2R)-1-(4-((S)-3-(aminomethyl)-1-hydroxy-1,3-dihydrobenzo[c][1,2]oxaborol-6-yl)phenyl)-1,3-dihydroxypropan-2-yl)-2-chloroacetamide;
N-[(5aR,6aS,7S,10aS)-9-carbamoyl-4,7-bis(dimethylamino)-1,8,10a,11-tetrahydroxy-10,12-dioxo-5,5a,6,6a,7,10,10a,12-octahydrotetracen-2-yl]-1-hydroxy-3,3-dimethyl-1,3-dihydro-2,1-benzoxaborole-6-carboxamide;
(4S,4aR,5S,5aR,6R,12aS)-4-(dimethylamino)-3,5,10,12,12a-pentahydroxy-9-({2-[(1-hydroxy-1,3-dihydro-2,1-benzoxaborol-6-yl)amino]-2-oxoethyl}amino)-6-methyl-1,11-dioxo-1,4,4a,5,5a,6,11,12a-octahydrotetracene-2-carboxamide;
N~1~-[(5R,5aR,6S,6aR,7S,10aS)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-N~4~-(1-hydroxy-1,3-dihydro-2,1-benzoxaborol-6-yl)butanediamide;
(4S,4aR,5S,5aR,6R,12aS)-4-(dimethylamino)-3,5,10,12,12a-pentahydroxy-9-({3-[(1-hydroxy-3,4-dihydro-1H-2,5,1-benzodioxaborepin-7-yl)amino]-3-oxopropyl}amino)-6-methyl-1,11-dioxo-1,4,4a,5,5a,6,11,12a-octahydrotetracene-2-carboxamide;
(4S,4aR,5S,5aR,6R,12aS)-9-({3-[(2,3-dihydro-7H-[1,2]oxaborolo[4,3,2-jk][2,5,1]benzodioxaborepin-9-yl)amino]-3-oxopropyl}amino)-4-(dimethylamino)-3,5,10,12,12a-pentahydroxy-6-methyl-1,11-dioxo-1,4,4a,5,5a,6,11,12a-octahydrotetracene-2-carboxamide;
quinolin-8-yl {3-chloro-4-[(dimethylamino)methyl]phenyl}(3-cyanophenyl)borinate; (4-fluorophenyl){3-[(4-methylpiperazin-1-yl)methyl]phenyl}(quinolin-8-olato-kappa~2~N,O)boron;
N-[(5R,5aR,6S,6aR,7S,10aS)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-1-hydroxy-3,3-dimethyl-1,3 dihydro-2,1-benzoxaborole-6-carboxamide;
(4S,4aR,5S,5aR,6R,12aS)-4-(dimethylamino)-3,5,10,12,12a-pentahydroxy-9-({3-[(1-hydroxy-3,4-dihydro-1H-2,1-benzoxaborinin-7-yl)amino]-3-oxopropyl}amino)-6-methyl-1,11-dioxo-1,4,4a,5,5a,6,11,12a-octahydrotetracene-2-carboxamide;
quinolin-8-yl (3-chloro-4-cyanophenyl){3-fluoro-4-[(4-methylpiperazin-1-yl)methyl]phenyl}borinate;
N~1~-[(5RS,5aRS,6SR,6aRS,7SR,10aSR)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-N~4~-{[(5RS)-2-hydroxy-1,2-oxaborolan-5-yl]methyl}butanediamide;
{3-chloro-4-[(4-methylpiperazin-1-yl)methyl]phenyl}(4-cyanophenyl)(quinolin-8-olato-kappa~2~N,O)boron;
dimethyl 4-(4,4,5,5-tetramethyl-1,3,2-dioxaborolan-2-yl)pyridine-2,6-dicarboxylate;
[(3S)-4-borono-1-hydroxy-1,3-dihydro-2,1-benzoxaborol-3-yl]aceticacid;
(4S,4aR,5S,5aR,6R,12aS)-4-(dimethylamino)-3,5,10,12,12a-pentahydroxy-9-{[N-(1-hydroxy-3,3-dimethyl-1,3-dihydro-2,1-benzoxaborol-6-yl)glycyl]amino}-6-methyl-1,11-dioxo-1,4,4a,5,5a,6,11,12a-octahydrotetracene-2-carboxamide;
quinolin-8-yl (3-chloro-4-cyanophenyl){4-[(4-methylpiperazin-1-yl)methyl]phenyl}borinate;
quinolin-8-yl (4-cyanophenyl){3-[(4-methylpiperazin-1-yl)methyl]phenyl}borinate;
N-[(6aS,10S,10aR,11S,11aR,12R)-8-carbamoyl-10-(dimethylamino)-4,6,6a,9,11-pentahydroxy-12-methyl-5,7-dioxo-5,6a,7,10,10a,11,11a,12-octahydrotetracen-1-yl]-1-hydroxy-3,3-dimethyl-1,3-dihydro-2,1-benzoxaborole-6-carboxamide;
N-(1-hydroxy-1,3-dihydro-2,1-benzoxaborol-6-yl)benzamide;
bis(acetato-kappaO)[1-cyclopropyl-6,7-difluoro-8-methyl-4-(oxo-kappaO)-1,4-dihydroquinoline-3-carboxylato-kappaO]boron;
N-[(6aS,10S,10aR,11S,11aR,12R)-8-carbamoyl-10-(dimethylamino)-4,6,6a,9,11-pentahydroxy-12-methyl-5,7-dioxo-5,6a,7,10,10a,11,11a,12-octahydrotetracen-1-yl]-1-hydroxy-1,3-dihydro-2,1-benzoxaborole-6-carboxamide;
N-(3-{[(5aR,6aS,7S,10aS)-9-carbamoyl-4,7-bis(dimethylamino)-1,8,10a,11-tetrahydroxy-10,12-dioxo-5,5a,6,6a,7,10,10a,12-octahydrotetracen-2-yl]amino}-3-oxopropyl)-1-hydroxy-1,3-dihydro-2,1-benzoxaborole-5-carboxamide;
quinolin-8-yl [3-(4,4-dimethyl-4,5-dihydro-1,3-oxazol-2-yl)phenyl](3-fluorophenyl)borinate;
{3-[(tert-butoxycarbonyl)amino]propyl}(trifluorido)borate(1-);
{3-[benzyl(methyl)amino]prop-1-en-2-yl}(trifluorido)borate(1-);
{(2Z)-3-(dimethylamino)-3-hydroxy-1-[4-hydroxy-2-(hydroxy-kappaO)phenyl]prop-2-en-1-onato-kappaO}(difluorido)boron;
5-phenoxy-2,1-benzoxaborol-1(3H)-ol;
bis(4-chlorophenyl)borinic acid;
(4-{[(3R)-1-(tert-butoxycarbonyl)piperidin-3-yl](3-methylpyridin-2-yl)carbamoyl}phenyl)(trifluorido)borate(1-);
N-[(3-methyl-1,2-oxazol-5-yl)methyl]-2-[(3S)-3-(2-methylphenyl)piperidin-1-yl]-7H-pyrrolo[2,3-d]pyrimidin-4-amine;
N-[(5R,5aR,6S,6aR,7S,10aS)-9-carbamoyl-7-(dimethylamino)-1,6,8,10a,11-pentahydroxy-5-methyl-10,12-dioxo-5,5a,6,6a,7,10,10a,12-octahydrotetracen-2-yl]-1-hydroxy-1,3-dihydro-2,1-benzoxaborole-6-carboxamide;
[(1Z)-1-(dimethylamino)-1-hydroxy-5-(hydroxy-kappaO)-4-phenylpenta-1,4-dien-3-onato-kappaO](difluorido)boron;
[3-(5-fluoro-2,1-benzoxaborol-1(3H)-yl)phenyl]methyl 8-hydroxyquinoline-2-carboxylate;
2-({[bis(3-fluorophenyl)boranyl]oxy}carbonyl)pyridin-3-ol;
3-methyl-4-(4,4,5,5-tetramethyl-1,3,2-dioxaborolan-2-yl)benzoic acid;
2-(4-bromophenyl)-5,6-dichloro-2H-1,3,2-benzodioxaborole; and
4,4′-{[1-(nonaboran-1-yl)-1H-borirene-2,3-diyl]bis(methylene)}diphenol, or a pharmaceutically acceptable salt thereof.

9. The composition according to claim 8, wherein the RNA is messenger RNA (mRNA).

10. The composition according to claim 9 wherein the mRNA encodes a viral antigen.


SEQUENCE LISTING (9 sequences)

SEQ ID NO: 1 - RNA, 454 nt, Artificial Sequence
  Synthetic nucleotide sequence: derived from HCV IRES
gccagcccccgauugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaccaugagcacgaauccuaaaccucaaagaaaaaccaaacguaacaccaaccgccgcccacaggacgucugaggcggccgccgccucagacguccuguggguuuauugcaucccgc

SEQ ID NO: 2 - DNA, 489 bp, Artificial Sequence
  Synthetic nucleotide sequence: derived from HCV IRES, with T7promoter and primer annealing site
ggatcctaatacgactcactatagccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtctgaggcggccgccgcctcagacgtcctgtgggtttattgcatcccgcctcgagtctaga

SEQ ID NO: 3 - RNA, 574 nt, Artificial Sequence
  Synthetic nucleotide sequence: derived from Trichoderma atroviride FgenI
ucucucgcuaucucggaaucgaggggucuggccuacgcugcgauugcuucuucagagcauccuucuucagccuugugcccucuacaguggcagccucacacaaacuaucaacauggcuuccgaauccaggcucuaccagaucuccggcgagaccaagucucaucugcucaaguuccgcuugacgacaucucgggccagcaaaccucaggcagugauuuauuugauugauaaaaacacucacgaaauccgccaagacgaugacaagacuguauacaccucccucgacgagauagccgacgaccuccccgacagcaccccucgauucaucuuccucagcuaucccuugacgaugcccgauggacggcuauccgugccuuacaccaugaucuacuaccuccccaucaacugcaaugccgcaacaaggaugcucuacgccggcgcaaaagagcugauacgcaacacggccgaggugaacaagguuaucgauauagagucugcagaggaucuggaagauauuccaaagcaguugagcggauagaaauaauaauaaaagaaauaacauguucgcuaugcc

SEQ ID NO: 4 - RNA, 612 nt, Artificial Sequence
  Synthetic nucleotide sequence: derived from Trichoderma atroviride FgenI, with primer annealing sites
uccaccaugaaucacuccucucucgcuaucucggaaucgaggggucuggccuacgcugcgauugcuucuucagagcauccuucuucagccuugugcccucuacaguggcagccucacacaaacuaucaacauggcuuccgaauccaggcucuaccagaucuccggcgagaccaagucucaucugcucaaguuccgcuugacgacaucucgggccagcaaaccucaggcagugauuuauuugauugauaaaaacacucacgaaauccgccaagacgaugacaagacuguauacaccucccucgacgagauagccgacgaccuccccgacagcaccccucgauucaucuuccucagcuaucccuugacgaugcccgauggacggcuauccgugccuuacaccaugaucuacuaccuccccaucaacugcaaugccgcaacaaggaugcucuacgccggcgcaaaagagcugauacgcaacacggccgaggugaacaagguuaucgauauagagucugcagaggaucuggaagauauuccaaagcaguugagcggauagaaauaauaauaaaagaaauaacauguucgcuaugccgaaaaaccaaacguaacacc

SEQ ID NO: 5 - DNA, 612 bp, Artificial Sequence
  Synthetic nucleotide sequence: derived from Trichoderma atroviride FgenI, with primer annealing sites
tccaccatgaatcactcctctctcgctatctcggaatcgaggggtctggcctacgctgcgattgcttcttcagagcatccttcttcagccttgtgccctctacagtggcagcctcacacaaactatcaacatggcttccgaatccaggctctaccagatctccggcgagaccaagtctcatctgctcaagttccgcttgacgacatctcgggccagcaaacctcaggcagtgatttatttgattgataaaaacactcacgaaatccgccaagacgatgacaagactgtatacacctccctcgacgagatagccgacgacctccccgacagcacccctcgattcatcttcctcagctatcccttgacgatgcccgatggacggctatccgtgccttacaccatgatctactacctccccatcaactgcaatgccgcaacaaggatgctctacgccggcgcaaaagagctgatacgcaacacggccgaggtgaacaaggttatcgatatagagtctgcagaggatctggaagatattccaaagcagttgagcggatagaaataataataaaagaaataacatgttcgctatgccgaaaaaccaaacgtaacacc

SEQ ID NO: 6 - RNA, 1667 nt, Artificial Sequence
  Synthetic nucleotide sequence: derived from Trichoderma atroviride Chi18-4
uacgccagacaauagauucagcucgaauaaugaagccgauuggucacugauuaacuugguguaucuacuggguagcaaaggaccgaucuucaccucgcaugaccguugaauguggcgcaauucuaguacuugaauggccuggccaagcuuauagaacaaucgagacgaauugaacaccacuaucaaaauccaaauuaugcgguuuucaaucauacauguggcuuuauggcuggccauggcggccgguucauuuacagcuaacgcuuacggagcuguucgaugcgucauguaucucacagggcaacacgugguagucccuucagaaccucaucucguggauuccauaacccauuugauacuggcuuuuaugcgcucugaugucuucaauguggacaaaacgccugcugaauucccgcucuucgcaucaguugcugagacgcgcgagaaguucaacgcaaauaccaagaucauggucgcaaucggcgguuggggagacgcaggauuugaagaagcugcgcgugacgauucaucgagaaagcggugggcuggccacgucaaggccaugguugaccagacaggagccgauggcauugacauugacugggaauauccgggagggaaccgugacgauuauaaacucauuccgaacucucagcgggaaugggagauucaggcauucgugcuccuucuucaagaauugcguuccgccuugggacaaacgaaacugcucucaauugcggugccagcgcuggaacgagauuugauggccuucacaaacucgacuauuccguccauuguggaucaaguugacuuuaucaguguaaugacuuacgacaugaugaaccgacgcgacaacguugucaagcaccacagcggcguggcugacuccugggaagcaaugaagcgauauauagaucgcggcgucccuccgcacaagcugaacuuuggacuugguuacuaugccaaaugguucaugacugaacaaugcgauguacagcacccauuaggaugccgcacucaacugcuagaagaccccgccaauggagccgaucuuggcaagacugcagcuuuuagcuggcacgacgagguucccgcagaaauggccaauuccuuuggcaaagcccaugcucauggccgcuacuaugaagauggaagcuacggcuauugggacgaugaagagaagagauggugguccuacgacacgccucucaccaucaaagccaaagugccucggcuucucggcgagcugcagcugggcgggguguuugccugggggcugggcgaggacgcuccgcaguuucugcaccugaaggcgacuguugacggcauucggguucugcgcggagaugacgaugcugugaaggaugagcuguaacaagcagcggcguguguacgucuggaguuuggcgacaucauugaaggcgugggaugcaauucaagccauugauacugcagauaaguugaagcagcaacagccaaaacucugcacguggcccgagacuuccgucuuauagcagggaacagacaggauggugcugacggaauaugcagagcucgagcucaaccgguaggcuagcuugaaaaccccaugagcuagcaguuaaccuugcaguuaaggguaaccagagcuuaaacacgucugauuuggugcaaugaauacaggcugc

SEQ ID NO: 7 - RNA, 1705 nt, Artificial Sequence
  Synthetic nucleotide sequence: derived from Trichoderma atroviride Chi18-4, with primer annealing sites
uccaccaugaaucacuccuacgccagacaauagauucagcucgaauaaugaagccgauuggucacugauuaacuugguguaucuacuggguagcaaaggaccgaucuucaccucgcaugaccguugaauguggcgcaauucuaguacuugaauggccuggccaagcuuauagaacaaucgagacgaauugaacaccacuaucaaaauccaaauuaugcgguuuucaaucauacauguggcuuuauggcuggccauggcggccgguucauuuacagcuaacgcuuacggagcuguucgaugcgucauguaucucacagggcaacacgugguagucccuucagaaccucaucucguggauuccauaacccauuugauacuggcuuuuaugcgcucugaugucuucaauguggacaaaacgccugcugaauucccgcucuucgcaucaguugcugagacgcgcgagaaguucaacgcaaauaccaagaucauggucgcaaucggcgguuggggagacgcaggauuugaagaagcugcgcgugacgauucaucgagaaagcggugggcuggccacgucaaggccaugguugaccagacaggagccgauggcauugacauugacugggaauauccgggagggaaccgugacgauuauaaacucauuccgaacucucagcgggaaugggagauucaggcauucgugcuccuucuucaagaauugcguuccgccuugggacaaacgaaacugcucucaauugcggugccagcgcuggaacgagauuugauggccuucacaaacucgacuauuccguccauuguggaucaaguugacuuuaucaguguaaugacuuacgacaugaugaaccgacgcgacaacguugucaagcaccacagcggcguggcugacuccugggaagcaaugaagcgauauauagaucgcggcgucccuccgcacaagcugaacuuuggacuugguuacuaugccaaaugguucaugacugaacaaugcgauguacagcacccauuaggaugccgcacucaacugcuagaagaccccgccaauggagccgaucuuggcaagacugcagcuuuuagcuggcacgacgagguucccgcagaaauggccaauuccuuuggcaaagcccaugcucauggccgcuacuaugaagauggaagcuacggcuauugggacgaugaagagaagagauggugguccuacgacacgccucucaccaucaaagccaaagugccucggcuucucggcgagcugcagcugggcgggguguuugccugggggcugggcgaggacgcuccgcaguuucugcaccugaaggcgacuguugacggcauucggguucugcgcggagaugacgaugcugugaaggaugagcuguaacaagcagcggcguguguacgucuggaguuuggcgacaucauugaaggcgugggaugcaauucaagccauugauacugcagauaaguugaagcagcaacagccaaaacucugcacguggcccgagacuuccgucuuauagcagggaacagacaggauggugcugacggaauaugcagagcucgagcucaaccgguaggcuagcuugaaaaccccaugagcuagcaguuaaccuugcaguuaaggguaaccagagcuuaaacacgucugauuuggugcaaugaauacaggcugcgaaaaaccaaacguaacacc

SEQ ID NO: 8 - DNA, 1705 bp, Artificial Sequence
  Synthetic nucleotide sequence: derived from Trichoderma atroviride Chi18-4, with primer annealing sites
tccaccatgaatcactcctacgccagacaatagattcagctcgaataatgaagccgattggtcactgattaacttggtgtatctactgggtagcaaaggaccgatcttcacctcgcatgaccgttgaatgtggcgcaattctagtacttgaatggcctggccaagcttatagaacaatcgagacgaattgaacaccactatcaaaatccaaattatgcggttttcaatcatacatgtggctttatggctggccatggcggccggttcatttacagctaacgcttacggagctgttcgatgcgtcatgtatctcacagggcaacacgtggtagtcccttcagaacctcatctcgtggattccataacccatttgatactggcttttatgcgctctgatgtcttcaatgtggacaaaacgcctgctgaattcccgctcttcgcatcagttgctgagacgcgcgagaagttcaacgcaaataccaagatcatggtcgcaatcggcggttggggagacgcaggatttgaagaagctgcgcgtgacgattcatcgagaaagcggtgggctggccacgtcaaggccatggttgaccagacaggagccgatggcattgacattgactgggaatatccgggagggaaccgtgacgattataaactcattccgaactctcagcgggaatgggagattcaggcattcgtgctccttcttcaagaattgcgttccgccttgggacaaacgaaactgctctcaattgcggtgccagcgctggaacgagatttgatggccttcacaaactcgactattccgtccattgtggatcaagttgactttatcagtgtaatgacttacgacatgatgaaccgacgcgacaacgttgtcaagcaccacagcggcgtggctgactcctgggaagcaatgaagcgatatatagatcgcggcgtccctccgcacaagctgaactttggacttggttactatgccaaatggttcatgactgaacaatgcgatgtacagcacccattaggatgccgcactcaactgctagaagaccccgccaatggagccgatcttggcaagactgcagcttttagctggcacgacgaggttcccgcagaaatggccaattcctttggcaaagcccatgctcatggccgctactatgaagatggaagctacggctattgggacgatgaagagaagagatggtggtcctacgacacgcctctcaccatcaaagccaaagtgcctcggcttctcggcgagctgcagctgggcggggtgtttgcctgggggctgggcgaggacgctccgcagtttctgcacctgaaggcgactgttgacggcattcgggttctgcgcggagatgacgatgctgtgaaggatgagctgtaacaagcagcggcgtgtgtacgtctggagtttggcgacatcattgaaggcgtgggatgcaattcaagccattgatactgcagataagttgaagcagcaacagccaaaactctgcacgtggcccgagacttccgtcttatagcagggaacagacaggatggtgctgacggaatatgcagagctcgagctcaaccggtaggctagcttgaaaaccccatgagctagcagttaaccttgcagttaagggtaaccagagcttaaacacgtctgatttggtgcaatgaatacaggctgcgaaaaaccaaacgtaacacc

SEQ ID NO: 9 - RNA, 30 nt, Artificial Sequence
  Synthetic nucleotide sequence
ggaaucucucucacgaacugacguaaucuu